(19) United States
(12) Patent Application Publication
(10) Pub. No.: US 2003/0211141 A1
(43) Pub. Date: Nov. 13, 2003

(54) Genetic and protein manipulation of betaIG-H3 for the treatment and cure of muscular dystrophies

(76) Inventors: Richard G. LeBaron, San Antonio, TX (US); Jill Whitelock Ferguson, Newton, MA (US)

Correspondence Address: ROBERT W STROZIER, P.L.L.C, PO BOX 429, BELLAIRE, TX, 77402-0429, US

(21) Appl. No.: 10/316,428

(22) Filed: Dec. 11, 2002

Related U.S. Application Data
(60) Provisional application No. 60/339,522, filed on Dec. 11, 2001.

(51) Int. Cl. A61K 48/00; A61K 9/127; A61K 39/395

(52) U.S. Cl. 424/450; 424/93.2; 514/12; 424/130.1

(57) ABSTRACT

Compositions and methods are disclosed for curing, treating or preventing the onset of Muscular Dystrophies or related neuromuscular diseases, where the compositions include βig-H3, a variant thereof.

### RELATED APPLICATIONS

This application claims provisional priority to U.S. Provisional Patent Application Serial No. 60/339,522 filed Dec. 11, 2001.

### BACKGROUND OF THE INVENTION

1. Field of the Invention

The present invention relates to compositions and method of administering the composition to prevent, treat or cure Muscular Dystrophies.

More particularly, the present invention relates to: 1) a composition including βIG-H3 or a variant thereof or a DNA sequence expressing βIG-H3 or a variant thereof for treating Muscular Dystrophies; 2) a method for treating patients with Muscular Dystrophies including administering a composition including βIG-H3 or a variant thereof or a DNA sequence expressing βIG-H3 or a variant thereof; 3) a method for ameliorating symptoms of Muscular Dystrophies including administering a composition including βIG-H3 or a variant thereof or a DNA sequence expressing βIG-H3 or a variant thereof according to a treatment protocol; 4) a method for preventing or delaying the onset of Muscular Dystrophies including administering a composition including βIG-H3 or a variant thereof or a DNA sequence expressing βIG-H3 or a variant thereof according to a preventative protocol; and 5) methods for using a composition including βIG-H3 or a variant thereof or a DNA sequence expressing βIG-H3 or a variant thereof to prevent, treat, or cure Muscular Dystrophies.

2. Description of the Related Art

Development of multicellular organisms is dependent on numerous and varied contacts of extracellular matrix (ECM) molecules and cells (Blaschuk, 1994). The ECM is comprised of collagens, proteoglycans, non-collagenous glycoproteins such as fibronectin, laminin, tenascin and likely yet-to-be discovered molecules. As new ECM molecules are investigated, information regarding their spatiotemporal expression is anticipated to provide a better understanding of their physiological function. Fairly recently, a gene responsive to transforming growth factor-β (TGF-β) was discovered by differential screening of an adenocarcinoma cDNA library ENRfu (Skonier et al., 1992). The newly identified gene, named Transforming Growth Factor-β Induced Gene-Human Clone 3 (βig-h3), encodes a 683 amino acid secretory protein that was designated βIG-H3 ENRfu (Skonier et al., 1992). βIG-H3 contains repeating units similar to recurring sequences found in fasciclin-I, a nerve cell growth cone guidance molecule expressed in developing Drosophila ENRfu (Zinn et al., 1988). Consensus sequences predicted to bind sulfated glycosaminoglycan ENRfu (Cardin and Weintraub, 1989) were discovered near the central portion of βIG-H3 and may be functional as βIG-H3 binds heparin-agarose (unpublished observation). Possibly mediating attachment to members of the integrin superfamily of cell surface adhesion receptors are the sequences Arg-Gly-Asp ENRfu (Pierschbacher and Ruoslahti, 1984), Asn-Lys-Asp-Ilu-Leu and Glu-Pro-Asp-Ilu-Met ENRfu (Kim et al., 2000b). Additionally, βIG-H3 binds collagens in vitro ENRfu (Hashimoto et al., 1997).

Immunochemistry and protein sequence analyses detected βIG-H3 in skin ENRfu (LeBaron et al., 1995), cornea ENRfu (Escribano et al., 1994; Hirano et al., 1996), bladder smooth muscle ENRfu (Billings et al., 2000) and as a component of elastic fibers ENRfu (Gibson et al., 1996). The distribution of βIG-H3 in adult tissues and the findings that βIG-H3 promotes cell adhesion ENRfu (Kim et al., 2000b; LeBaron et al., 1995) and binds to collagens ENRfu (Hashimoto et al., 1997; Rawe et al., 1997) and heparin suggests that βIG-H3 functions in development and tissue modeling, interacting with cells and ECM molecules. The βIG-H3 gene maps to human chromosome 5q31, a region proposed to contain genes that when mutated, then may play a pathogenic role, contributing toward the development of tumors and corneal and muscular dystrophies (see discussion). However, the normal physiologic function of βIG-H3 and mechanisms that may mediate its possible role in pathogenicities in vivo are not clear.

Muscular dystrophies are Neuromuscular Diseases including: Duchenne Muscular Dystrophy (DMD) (Pseudohypertrophic), Becker Muscular Dystrophy (BMD), Emery-Dreifuss Muscular Dystrophy (EDMD), Limb-Girdle Muscular Dystrophy (LGMD), Facioscapulohumeral Muscular Dystrophy (FSH or FSHD) (Landouzy-Dejerine), Myotonic Dystrophy (MMD) (Steinert's Disease), Oculopharyngeal Muscular Dystrophy (OPMD), Distal Muscular Dystrophy (DD) (Miyoshi), Congenital Muscular Dystrophy (CMD). Related diseases including Motor Neuron Diseases such as Amyotrophic Lateral Sclerosis (ALS) (Lou Gehrig's Disease), Infantile Progressive Spinal Muscular Atrophy (SMA, SMA1 or WH)(SMA Type 1, Werdnig-Hoffman), Intermediate Spinal Muscular Atrophy (SMA or SMA2)(SMA Type 2), Juvenile Spinal Muscular Atrophy (SMA, SMA3 or KW)(SMA Type 3, Kugelberg-Welander), Spinal Bulbar Muscular Atrophy (SBMA)(Kennedy's Disease and X-Linked SBMA), and Adult Spinal Muscular Atrophy (SMA) and Diseases of the Neuromuscular Junction such as Myasthenia Gravis (MG), Lambert-Eaton Syndrome (LES), and Congenital Myasthenic Syndrome (CMS). Although many researchers have worked and continue to work on new treatments and possible cures to these diseases, most of these diseases are still relatively untreatable and difficult to manage therapeutically.

Thus, there is a need in the art for new and advanced composition for preventing, treating or curing Muscular Dystrophies and methods for preventing, treating or curing Muscular Dystrophies.

### SUMMARY OF THE INVENTION

The present invention provides a composition including βig-H3 or a variant thereof or a DNA sequence expressing βIG-H3 or a variant thereof for treating Muscular Dystrophies and related neuromuscular diseases.

The present invention provides a method for treating patients with Muscular Dystrophies or related neuromuscular diseases including administering a composition including βIG-H3 or a variant thereof or a DNA sequence expressing βIG-H3 or a variant thereof.

The present invention provides a method for ameliorating symptoms of Muscular Dystrophies or related neuromuscular diseases including administering a composition including βIG-H3 or a variant thereof or a DNA sequence expressing βIG-H3 or a variant thereof according to a treatment protocol.

The present invention provides a method for preventing or delaying the onset of Muscular Dystrophies or related neuromuscular diseases including administering a composition including βIG-H3 or a variant thereof or a DNA sequence expressing βIG-H3 or a variant thereof according to a preventative protocol.

The present invention provides methods for using a composition including βIG-H3 or a variant thereof or a DNA sequence expressing βIG-H3 or a variant thereof to prevent, treat, or cure Muscular Dystrophies or related neuromuscular diseases

### DESCRIPTION OF THE DRAWINGS

The invention can be better understood with reference to the following detailed description together with the appended illustrative drawings in which like elements are numbered the same:

FIG. 1 depicts schematic of βIG-H3. Illustrated is the protein βIG-H3 showing internal repeats I-IV that contain limited homology to fasciclin I. The approximate locations of consensus sequences reported to mediate cell adhesion (Asn-Lys-Asp-Ilu-Leu and Glu-Pro-Asp-Ilu-Met) and an Arg-Gly-Asp tripeptide sequence are included. The shaded regions in repeat III indicate the putative heparin-binding sequences Lys-Lys-Leu-Arg and Lys-Arg-Gly-Arg. The asterisk denotes the region within βIG-H3 corresponding to nucleotide sequence that was used as an in situ hybridization probe.

FIG. 2 depicts transcripts of βIG-H3 are expressed in pre-chondrocytic mesenchymal cells in areas of axial, craniofacial, and appendicular primordial cartilage. As early as embryonic day 12.5, βIG-H3 transcripts were expressed by mesenchymal cells recruited to the regions of future bone (transverse sections). Darkfield (A, D, G, J) and brightfield (C, F, I, L) photomicrographs indicate the regions where βIG-H3 transcripts were detected at areas of cell condensation. E13.5 vertebral bone is denoted by arrows and the notochord identified by double arrowheads (A, C). E13.5 rib cartilage primordia expressed βIG-H3 transcripts (D and F, asterisk). Additional areas of expression included E13.5 upper limb cartilage (G and I, area of expression defined by arrows). An increased magnification shows βIG-H3 transcripts in E14.5 nasal cartilage primordia (J and L, asterisk). Representative sense controls (B, E, H, K). Scale bars represent 50 μm.

FIG. 3 depicts growth plates in bone tissue express βIG-H3. Transverse sections show βIG-H3 expression was evident in proliferating chondrocytes including E15.5 cranial (A, C, asterisk) and E14.5 vertebral growth plates (D, F, asterisk). Representative sense controls (B, E). Scale bars represent 50 μm.

FIG. 4 depicts βIG-H3 transcript localization to regions of proliferating chondrocytes and areas of ossification. Darkfield (A, D, G, J) and brightfield (C, F, I, L) images indicate the localization of βIG-H3 to regions of proliferating chondrocytes. An E16.5 scapula is shown (A and C, sagittal sections). βIG-H3 expression was also evident in the initiation of endochondral ossification in developing limb bones (D), here shown by an asterisk in an E17.5 tibia (F). Areas of intramembranous ossification also displayed βIG-H3 transcripts (G). βIG-H3 transcripts are detected around the temporal lobe of the embryonic brain at E17.5 (I, arrows) but not in the brain per se (I, double asterisk). Sagittal sections of E17.5 vertebral column revealed that the dura mater displayed βIG-H3 transcripts (J, arrows in L). Marked βIG-H3 expression was observed in regions of proliferating chondrocytes. Areas devoid of βIG-H3 message correspond to regions of hypertrophic stage chondrocytes (L, double arrowheads) and the spinal cord (L, asterisk). Representative sense controls (B, E, H, K). Scale bars represent 50 μm.

FIG. 5 depicts βIG-H3 transcripts in joint tissue. βIG-H3 transcripts were observed in transverse sections of developing joint region, including articular cartilage between the cartilage primordia of a developing mouse footpad (A, C, arrows) and between cartilage regions in an E18.5 hindlimb (D, F, arrows). Representative sense controls (B, E). Scale bars represent 50 μm.

FIG. 6 depicts βIG-H3 transcript localization to connective tissue capsules. Transverse sections reveal βIG-H3 message in connective tissue capsules. Areas expressing βIG-H3 message include the capsule of an E17.5 kidney (A, C, arrows), as well as the connective tissue surrounding the glomeruli within the medulla (A, C, double asterisk). Adjacent to the kidney is the ovary (C, single asterisk) expressing moderate levels of βIG-H3. Another connective tissue, the pleural pericardium membrane moderately expressed βIG-H3 transcripts (D, F arrows) from E12.5-E15.5 (E14.5 shown). Strong βIG-H3 message was also observed in the region of heart valve formation (F, asterisk). βIG-H3 expression localized to the connective tissue capsule tunica albuginea in E17.5 testes (G, I arrows) and especially evident at the rete testis and the mediastinum (G, I, arrowheads). The serosa and muscularis externa layers (J, L arrows) of the digestive tract, as well as the lamina propia (J, L, arrowheads) of the E17.5 intestinal tract displayed βIG-H3 transcripts. Representative sense controls are indicated in (B, E, H, K). Scale bars represent 50 μm.

FIG. 7 depicts βIG-H3 transcripts localize to areas of epithelial-mesenchymal interactions. Transverse sections (A, C) show the corneal epithelium (arrowheads) and stroma (arrows) from E16.5-E18.5 (E16.5 shown) and mesenchyme in developing vibrissae (D, F, arrows) from E14.5-E18.5 (E18.5 shown). Moderate expression of βIG-H3 mRNA was detected in the epithelium surrounding the hair follicle (D, F, double arrowheads). βIG-H3 transcripts were also detected in the surrounding mesenchyme of developing cochlea from E13.3-E18.5 (G, I, arrows; E17.5 shown). βIG-H3 expression was evident in the mesenchyme surrounding the epithelial layer of the developing cartilaginous bronchi of the lungs (J, L; single arrowhead) from E12.5 until E18.5, and in the cartilage surrounding the trachea (J, L, double arrow, E15.5 shown). Expression was also observed in the mesenchyme and smooth muscle layers surrounding the aorta (J, L, single arrow) and esophagus (J, L, double arrowhead). Representative sense controls (B, E, H, K). Scale bars represent 50 μm.

FIG. 8 depicts βIG-H3 expression in the dura mater and trigeminal ganglia. βIG-H3 transcripts localize to the dura mater (A, C, arrow) surrounding the developing optic nerve stalk (asterisk). Expression was observed from E14.5 to E18.5 (E17.5 shown). Additionally, the sclera of the eyeball expressed βIG-H3 transcripts (A, C, double arrowhead). Transcripts were observed throughout the trigeminal ganglia (D), with an apparent increased signal density in the caudal half of the trigeminal ganglia (D, F arrow) (E14.5 shown). Rathke's pouch displayed βIG-H3 message (G), localizing to areas of cellular and vascular proliferation surrounding the lumen (I, arrows) (E14.5 shown). Representative sense controls (B, E, H). Scale bars represent 50 μm.

FIG. 9 depicts βIG-H3 expression in muscle tissue. βIG-H3 transcripts were expressed in the epimysium (A, C, arrows) surrounding muscle fiber bundles at E17.5 and over the entire area of the diaphragm (D, F, arrows), remaining constant in diaphragm expression levels from E15.5-E17.5 (E17.5 shown). In early cardiac muscle tissue, βIG-H3 expression was abundant in cardiac valve formation (G, I). E14.5 heart valves (I, single arrow) are near a developing rib (I, double arrowhead). Representative controls sections are illustrated in (B, E and H). All results listed above are transverse sections. Scale bars represent 50 μm.

FIG. 10 depicts βIG-H3 promoted the attachment of mesenchymal cells. Cell attachment assays included human dermal fibroblasts, C2C12 murine skeletal muscle myoblasts, primary murine myoblasts, 2T3 murine osteoblasts, SV-40-transfected rabbit corneal epithelial cells (CECL), and a rabbit TRK-43 stromal fibroblast cell line. A second stromal keratocyte cell line, TRK-36, displayed the characteristic wounded phenotype of keratocytes (constitutively expressing—smooth actin) and was also tested. Substrata were formed by coating wells with 10 μg/ml of respective protein and each well seeded with 4×10⁴ cells. Mesenchymal-derived cell types attached to βIG-H3 while few, if any corneal epithelial cells attached to βIG-H3. βIG-H3 (closed bars), type I collagen (stripped bars), and BSA (open bars). Average values±S.D. are from three separate experiments.

FIG. 11 depicts Schematic illustrating the approximate locations of repetitive and consensus sequences in βIG-H3. The fasciclin 1-like repeats are designated I-IV. Closed bars at the amino and carboxyl terminals indicate sequence outside of the repeating units. Thin closed bars between each repeating unit are included to clarify individual fasciclin 1-like repeats. Peptide sequences proposed to play a role in cell attachment are localized within repeat II (NKDIL) and IV (EPDIM and RGD). Localization of heparin-binding consensus sequence is designated by the striped region within the third repeat. The dashed line approximates the portion that corresponding to nucleotide sequence utilized to develop an RNA probe.

FIG. 12 depicts βIG-H3 mRNA transcripts localize to the developing myotendinous junction (MTJ). Darkfield and brightfield photomicrographs of E16.5 sagittal sections of scapula indicate βIG-H3 transcripts at MTJs of developing mouse embryo tissue proximal to scapula cartilage primordia (arrow, A-C). Developing muscle fibers and tendon stained with Masson's trichrome indicate MTJs near an E16.5 scapula (arrow, C). MTJs at E17.5 rib cartilage primordia expressed βIG-H3 (arrows, D-F). The center rib cartilage primordia (shown in D) is magnified in (F). MTJs at an E17.5 femur are indicated (arrow, G, H, transverse sections). The asterisk signifies a region of the femur containing proliferating chondrocytes expressing βIG-H3 transcripts. Scale bars represent 50 μm.

FIG. 13 depicts Muscle fiber termini at MTJs stain distinctly with anti-βIG-H3 antibody. Anti-βIG-H3 antibody localized to the termini of muscle fibers (arrows, A, B, C, and E). Shown is staining at the E17.5 femur (asterisk, A) and discrete staining distal to the MTJ (arrowhead, A). βIG-H3 was observed within E18.5 developing facial bone (asterisk, B) located near the optic nerve (B), and βIG-H3 adjacent to the perichondrium of developing E17.5 rib (arrows, C). A serial section of the tissue shown in ‘C’ was treated with anti-βIG-H3 antibody pre-absorbed with recombinant βIG-H3 (D). Increased magnification revealed that βIG-H3 localizes to the apparent contacts of E17.5 rib myofibers (arrows, E). A serial section (F) stained with anti-myosin antibody shows that fibers contain myosin (sagitally-cut, arrow; cross-sectioned, arrowhead, F). Scale bars represent 50 μm.

FIG. 14 depicts Extracellular ultrastructural localization of βIG-H3 at MTJs. Indirect immunochemical ultrastructural analysis of transverse sections revealed that βIG-H3 is localized at or near fibers throughout the extracellular space surrounding myoblasts at an E17.5 developing MTJ (A), increased magnification (B). Arrows indicate the cell edge, arrowheads indicate extracellular fibers. Nucleus (N); Scale bar in A and B represent 1 and 0.2 μm, respectively.

FIG. 15 depicts Synthesis of βIG-H3 by C2C12 myoblasts is responsive to treatment with TGF-β1. An increase in βIG-H3 was observed in growth medium conditioned by C2C12 myoblasts treated with TGF-β1. Medium from cells treated with 20 ng/ml TGF-β1 for 24 hours shows prominent Coomassie Brilliant Blue staining within a region containing molecules that migrated at 68 kDa (A, lane 1, arrow). Medium conditioned by cells without addition of TGF-β1 exhibited less staining within the 68 kDa region (A, lane 2). A protein blot demonstrates that media shown in lanes 1 and 2 contained a similar disparate distribution of βIG-H3 (A, lanes 3 and 4, respectively). Material loaded on each lane was normalized to cell number. C2C12 myoblasts stained with anti-βIG-H3 antibody revealed that βIG-H3 appears deposited at the edges of cells. Shown are cells cultured in DMEM containing 10% serum (B). Scale bar represents 10 μm.

FIG. 16 depicts Purification of human recombinant βIG-H3 utilizing column chromatography. CHO cell conditioned medium contains recombinant βIG-H3 as illustrated by SDS-PAGE and visualized with Coomassie Brilliant Blue R250 (A, lane 1) and a protein immunoblot (B, lane 1). Material (e.g., lane 1) was applied on an anion exchange column. Material eluted that contained βIG-H3 (boxed peak in C; lane 2, A and B) was applied over hydroxyapatite. The material eluted that contained βIG-H3 (boxed peak in D; lane 3, A and B) was applied onto heparin-agarose where βIG-H3 eluted is in the boxed peak (E). A single band eluted from heparin resin was observed at 68 kDa and reacted with anti-βIG-H3 antibody (lane 4, A and B).

FIG. 17 depicts Attachment of C2C12 myoblasts to βIG-H3 is dependent on the concentration of βIG-H3 in the substratum and on the time of incubation. Wells were coated with various concentrations of βIG-H3 (A) or with 30 μg/ml βIG-H3 (B). Substrata were seeded with 4×10⁴ cells in serum-free medium and incubated at 37° C. for 60 minutes (A) or the times indicated (B). At the endpoints, non-attached cells were rinsed from the wells and the number of cells that remained attached quantified. To prevent endogenous protein synthesis from affecting the results, cycloheximide was included in the adhesion experiment (see methods section).

FIG. 18 depicts C2C12 myoblasts spread on a substratum of βIG-H3. Cells (4×10⁴) in serum-free medium were placed into wells coated with various substrata. After a 60-minute incubation at 37° C., cell attachment and spreading were documented. Shown in (A) is a graphical representation of cell attachment on the following substrata; βIG-H3, Coll (type I collagen), Fn (fibronectin), and BSA. Photomicrographs show myoblasts spread on substrata comprised of 10 μg/ml of βIG-H3 (B), type I collagen (C) and fibronectin (D). Myoblasts were treated with 10 μg/ml cycloheximide for one hour prior to and throughout the assay. Average values±S.D. are from three separate experiments. Scale bar represents 50 μm.

FIG. 19 depicts Primary skeletal myoblasts attach to a substratum of βIG-H3. Skeletal myoblasts (4×10⁴ cells/well) isolated from E17.5 mouse quadricep muscle were seeded on βIG-H3, Coll (type I collagen), Fn (fibronectin) and BSA. Myoblasts attached to βIG-H3, type I collagen and fibronectin. Few, if any myoblasts attached to BSA. Myoblasts were treated with 10 mg/ml cycloheximide for one hour prior to and throughout the assay. Average values±S.D. are from three separate experiments.

FIG. 20 depicts Characterization of C2C12 myoblast attachment to βIG-H3. Attachment specificity was demonstrated by pre-incubating myoblasts in a solution of serum-free medium containing a suspension of 30 μg/ml βIG-H3. Pre-incubation times are given on the ordinate (A). At the appropriate time, cells were seeded in microtiter wells coated with 30 μg/ml βIG-H3. Non-attached cells were removed 60 minutes after initial seeding and the cells remaining attached quantified (A). For comparison to different substrata, cells pre-incubated 30 minutes with βIG-H3 were seeded onto substrata comprised of 10 μg/ml βIG-H3 (B), fibronectin (C) or laminin (D). Cells pre-incubated in a solution containing βIG-H3 (stripped bars) had reduced competence to attach to βIG-H3 (B) when compared to cells pre-incubated without βIG-H3 (closed bars) under otherwise identical times and conditions (B). However, little, if any reduction in the number of attached cells was detected when cells pre-incubated with βIG-H3 were seeded onto fibronectin (C, striped bar) and laminin (D, striped bar). Pre-incubation of C2C12 myoblasts with increasing concentrations of EDTA resulted in a decreased number of cells attached to a βIG-H3 substratum (E). Results show values±S.D. derived from the average of duplicate wells/experiment from two separate experiments.

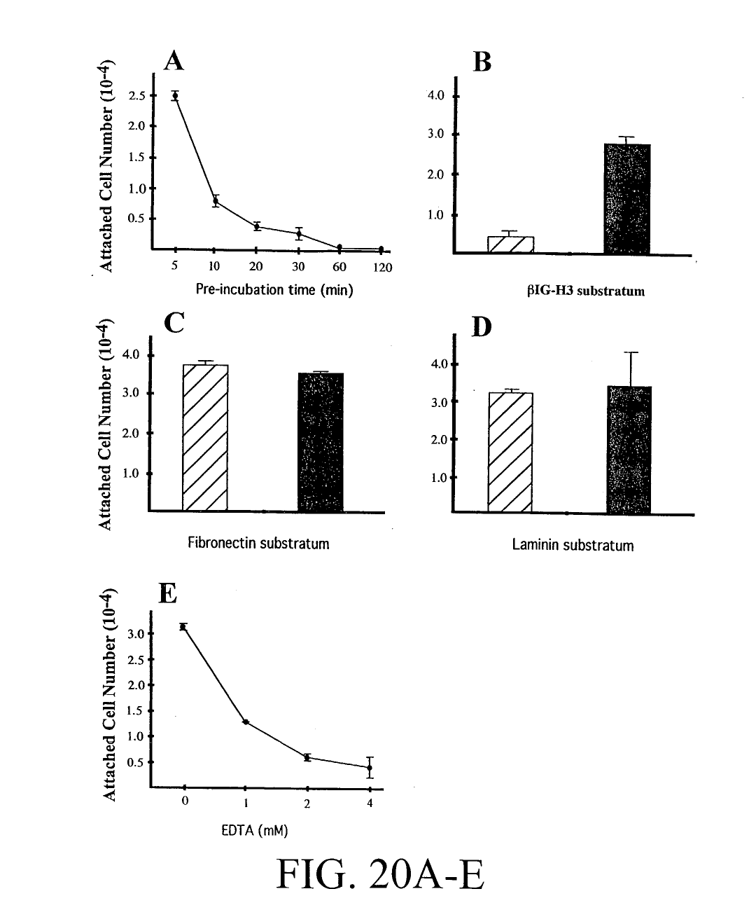

FIG. 21 depicts Skeletal muscle attachment to βIG-H3 is inhibited by function-antagonizing anti-integrin α7β1 antibody. C2C12 myoblasts (4×10⁴ cells/well) were pre-incubated with specific function-antagonizing anti-integrin antibodies for 30 minutes prior to their seeding on a βIG-H3 substratum comprised of 10 μg/ml. Cells that were pre-incubated with anti-α7 and anti-β1 antibodies (1:200 dilution) exhibited a significant reduction in their attachment to βIG-H3 (p≦0.008, β1; p≦0.04, α7). Other function perturbing antibodies for various integrin subunits did not significantly reduce the number of cells attached, nor did pre-incubation of cells with normal IgG (A). To compare perturbation of α7β1-mediated cell attachment onto a substratum of laminin (10 μg/ml stripped bars) and a substratum comprised of βIG-H3 (10 μg/ml, closed bars), cells were pre-incubated with antibody to α7 and to β1, and a mixture of both antibodies, prior to seeding (B). Average values±S.D. are from three separate experiments with duplicate wells in each experiment.

FIG. 22 depicts A gene delivery system is shown using antisense DNA using adeno-associated viral vectors, one of many DNA delivery systems.

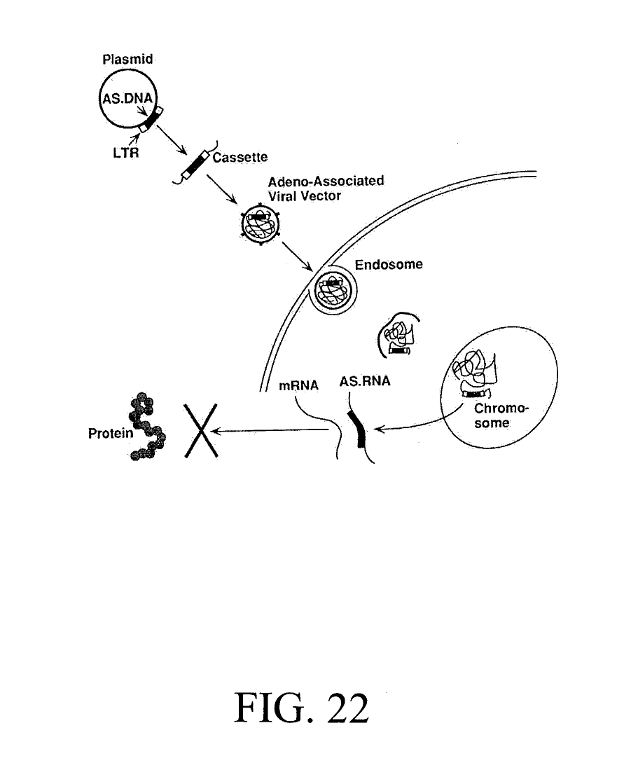

### DETAILED DESCRIPTION OF THE INVENTION

The inventors have found that compositions including βIG-H3 or variants thereof or DNA encoding βIG-H3 or variants thereof can be prepared and used to prevent, treat, or cure Muscular Dystrophies or related neuromuscular diseases.

One preferred aspect of this invention broadly relates to a composition comprising a therapeutically effective amount of βIG-H3, a βIG-H3 variant, a portion of βIG-H3, a variant of a portion βIG-H3 or mixtures thereof, where the amount is sufficient to cure, treat, ameliorate, and/or prevent symptoms of Muscular Dystrophies or related neuromuscular diseases. Preferably, the portion of βIG-H3 or a variant thereof is capable of eliciting a therapeutically beneficial response in patients with Muscular Dystrophies or related neuromuscular diseases symptoms.

Another preferred aspect of this invention broadly relates to a composition comprising an amount of a DNA sequence encoding βIG-H3, a DNA sequence encoding a βIG-H3 variant, a DNA sequence encoding a portion of βIG-H3, a DNA sequence encoding a portion of a βIG-H3 variant, antisense sequences corresponding thereto, or mixtures thereof, where the amount is sufficient to cause expression of the sequences in cells of an animal including a human to produce a therapeutically effective amount of encoded polypeptides sufficient to ameliorate, treat, prevent and/or cure Muscular Dystrophies or related neuromuscular diseases. Preferably, the sequences encoding a portion of βIG-H3 or a variant thereof are encoding portions that are capable of eliciting a therapeutically beneficial response in patients with Muscular Dystrophies or related neuromuscular diseases symptoms.

Another preferred aspect of this invention broadly relates to A plasmid comprising a DNA sequence encoding βIG-H3, a DNA sequence encoding a βIG-H3 variant, a DNA sequence encoding a portion of βIG-H3, a DNA sequence encoding a portion of a βIG-H3 variant, antisense sequences corresponding thereto, or mixtures thereof. Preferably, the plasmid elicits a therapeutic beneficial response to cure, treat, ameliorate, or prevent symptoms of Muscular Dystrophies or related neuromuscular diseases, when administered to an animal including a human in a therapeutically sufficient amount.

Another preferred aspect of this invention broadly relates to a DNA delivery system comprising a plasmid comprising a DNA sequence encoding βIG-H3, a DNA sequence encoding a βIG-H3 variant, a DNA sequence encoding a portion of βIG-H3, a DNA sequence encoding a portion of a βIG-H3 variant, antisense sequences corresponding thereto, or mixtures thereof. Preferably, the DNA delivery system elicits a therapeutic beneficial response to cure, treat, ameliorate, or prevent symptoms of Muscular Dystrophies or related neuromuscular diseases, when administered to an animal including a human in a therapeutically sufficient amount. Preferred DNA delivery systems include viral DNA delivery systems or a liposome DNA delivery systems or mixtures thereof.

Another preferred aspect of this invention broadly relates to a method for treating Muscular Dstrophies or related neuromuscular diseases comprising the step of administering to a patient a therapeutically effective amount of a composition including βIG-H3, a βIG-H3 variant, a portion of βIG-H3, a variant of a portion βIG-H3 or mixtures thereof, where the amount it sufficient to reduce, prevent, cure, and/or treat symptoms associated with Muscular Dstrophies or related neuromuscular diseases.

Another preferred aspect of this invention broadly relates to a method for treating Muscular Dstrophies or related neuromuscular diseases comprising the step of administering to a patient a composition comprising a DNA sequence encoding βIG-H3, a DNA sequence encoding a βIG-H3 variant, a DNA sequence encoding a portion of βIG-H3, a DNA sequence encoding a portion of a βIG-H3 variant, antisense sequences corresponding thereto, or mixtures thereof in an amount sufficient to cause cells in the patient to express a translated polypeptide corresponding to the sequences at a therapeutically effective level to reduce, prevent, cure, ameliorate, and or treat symptoms of Muscular Dstrophies or related neuromuscular diseases. Preferred composition are plasmids, a DNA delivery system such as a viral delivery system or a liposome delivery system or mixture thereof.

Another preferred aspect of this invention broadly relates to a method for delaying the onset of Muscular Dystrophies or related neuromuscular diseases comprising administering to a patient a composition comprising βIG-H3, a βIG-H3 variant, a portion of βIG-H3, a variant of a portion βIG-H3 or mixtures thereof according to a prophylactic treatment protocol sufficient to prevent or delay the onset of symptoms of Muscular Dystrophies or related neuromuscular diseases. Preferably, the the protocol comprises periodic administration of an amount of the composition at a level sufficient to prevent or delay the onset of symptoms of Muscular Dystrophies or related neuromuscular diseases or the protocol comprises continuous administration of an amount of the composition at a level sufficient to prevent or delay the onset of symptoms of Muscular Dystrophies or related neuromuscular diseases.

In administering the compositions of this invention to patients to treat the symptoms of Muscular Dystrophies or related neuromuscular diseases or to outright cure the diseases, the administration, when done on a periodic regiment, will generally have a period of the is less than a time required for the composition to no long treat, cure, prevent or delay the onset of symptoms of Muscular Dystrophies or related neuromuscular diseases. Preferably, the period of the periodic administration is less than or equal to six months, less than or equal to 3 months, less than or equal to 1 month, less than or equal to 2 weeks, less than or equal to 1 week or less than or equal to 1 day depending on the severity of the symptoms.

When administering the protein compositions of this invention a therapeutically effective amount of protein or protein fragment (portion of the protein) is generally between about 0.001 mg/Kg of body weight and about 10,000 mg/Kg of body weight. Preferably, the amount is between about 0.01 mg/Kg of body weight and about 5,000 mg/Kg of body weight. Particularly, the amount is between about 0.1 mg/Kg of body weight and about 1,000 mg/Kg of body weight, and more particularly, between about 1 mg/Kg of body weight and about 500 mg/Kg of body weight.

### SCOPE OF INVENTION SECTION

Muscular Dystrophy encompasses several distinct forms characterized by clinical phenotypes. The identification of the protein Dystrophin and the Dystrophin-Glycoprotein complex (DGC) located at the membrane of the myofiber was a first step towards characterizing muscular dystrophies based on molecular pathogenesis (Cohn and Campbell, 2000). A large number of genes are involved in the different forms of muscular dystrophy, many encoding for the DGC components which normally link the intracellular cytoskeleton to the extracellular matrix. Mutations in these components are thought to lead to loss of sarcolemmal integrity and render muscle fibers more susceptible to damage and necrosis, the major event in muscular dystrophy.

Mutations in other proteins outside of the DGC are being discovered that also contribute to various dystrophy phenotypes. Laminin α2 (Helbling-Leclerc et al., 1995) and α7 integrin subunit mutations (Miosge et al., 1999) were found to interfere with the normal integrity of the muscle fibers and the MTJ. Evidence suggests the DGC may be more important for lateral integrity of the myofibers, and the α7β1 integrin has a more important binding function at the MTJ (Cohn and Campbell, 2000). This evidence indicates that myofibers need two separate but parallel attachment systems for anchorage-dependent stability and survival—the DGC and the α7β1 integrin (Cohn and Campbell, 2000). Both systems confer overall stability and ECM cell survival and signaling.

On the morphological level of the MTJ, myofibers insert into protein plaque densities at the MTJ and folding of the junctional membrane occurs from embryo day 15-18 (Tidball and Lin, 1989). This invention presented the novel localization of βIG-H3 to the embryonic MTJ during this same time period of development, with prominent localization of βIG-H3 on the surface of myofibers as well as part of the surrounding ECM composition. This observation was extended to the molecular level by revealing βIG-H3 interacts with C2C12 myoblasts through the α7β1 integrin in vitro, indicative of what may possibly occur at the developing MTJ (attached manuscripts). The finding that βIG-H3 promotes the attachment of several mesenchymal cell types, including skeletal muscle cells, suggests βIG-H3 may serve a purpose in tissue genesis by promoting adhesive interactions with other protein or carbohydrate components of the extracellular matrix. The present invention is based on a potential and novel disease etiology that is responsible for a previously undefined member of the congenital and Limb Girdle Type 1A muscular dystrophy family. The novel etiology was identified through a previous study of levels of the βIG-H3 protein in mammalian muscle tissue.

There are two dystrophic phenotypes that potentially involve βIG-H3 based on the inventors work. Since results in this study suggest βIG-H3 binds the α7β1 integrin on skeletal muscle cell, α7 mutations found in patients with Congenital Muscular Dystrophy (CMD) is likely to affect βIG-H3 interaction with the receptor and subsequent intracellular signaling and structural properties of the muscle cell. CMD is an autosomal recessive disease found in both males and females beginning at birth and involves a generalized weakness of facial and limb muscles. Documented ultrastructural analysis revealed that myotendinousjunctions (MTJs) of α7-deficient mice lose their interdigitations and the myofilaments retract from the sarcolemmal membrane (Miosge et al., 1999). Based on previous ultrastructure results, muscle fiber separation from the surrounding extracellular matrix potentially occurs if the α7 mutation affected βIG-H3-α7 integrin interactions.

Limb Girdle Muscular Dystrophy is another dystrophic form linked to protein mutations. Limb Girdle Dystrophy is autosomal recessive in males and females and begins in early adolescence or adulthood. Progressive weakness in the hips and shoulder girdles, as well as absent or reduced tendon reflexes, is the clinical manifestations of this form. The Type 1A form of this dystrophy has been shown to involve mutations isolated to the 5q31 chromosome (Bartoloni, 1998 #370). This information is intriguing since βIG-H3 mutations have been linked to several corneal dystrophies, all linked to the chromosome 5q31 as well (Munier et al., 1997). This invention is directed at mutations on chromosome 5q31 (Bartoloni et al., 1998) which may be βIG-H3 mutations and that these mutations form or contribute to the molecular basis of the LGMD type 1A and congenital MD clinical phenotypes. The following experiments investigate βIG-H3 mutant roles in muscular dystrophies, specifically Limb Girdle MD Type 1A and Congenital MD.

The GenBank accession number for the known βIG-H3 genomic sequences are as follows: human M77349, mouse L19932, and rabbit U66205, the GenBank sequences for the genes and their corresponding protein amino acid sequences are included in Tables I, II, and III, respectively.

TABLE I

Human βig-h3 Gene Sequence and Corresponding βIG-H3 Protein Sequence
Human βig-h3 Gene Sequence

gcttgcccgt cggtcgctag ctcgctcggt gcgcgtcgtc ccgctccatg gcgctcttcg tgcggctgct
ggctctcgcc ctggctctgg ccctgggccc cgccgcgacc ctggcgggtc ccgccaagtc gccctaccag
ctggtgctgc agcacagcag gctccggggc cgccagcacg gccccaacgt gtgtgctgtg cagaaggtta
ttggcactaa taggaagtac ttcaccaact gcaagcagtg gtaccaaagg aaaatctgtg gcaaatcaac
agtcatcagc tacgagtgct gtcctggata tgaaaaggtc cctggggaga agggctgtcc agcagcccta
ccactctcaa acctttacga gaccctggga gtcgttggat ccaccaccac tcagctgtac acggaccgca
cggagaagct gaggcctgag atggaggggc ccggcagctt caccatcttc gcccctagca acgaggcctg
ggcctccttg ccagctgaag tgctggactc cctggtcagc aatgtcaaca ttgagctgct caatgccctc
cgctaccata tggtgggcag gcgagtcctg actgatgagc tgaaacacgg catgaccctc acctctatgt
accagaattc caacatccag atccaccact atcctaatgg gattgtaact gtgaactgtg cccggctcct
gaaagccgac caccatgcaa ccaacggggt ggtgcacctc atcgataagg tcatctccac catcaccaac
aacatccagc agatcattga gatcgaggac acctttgaga cccttcgggc tgctgtggct gcatcagggc
tcaacacgat gcttgaaggt aacggccagt acacgctttt ggccccgacc aatgaggcct tcgagaagat
ccctagtgag actttgaacc gtatcctggg cgacccagaa gccctgagag acctgctgaa caaccacatc
ttgaagtcag ctatgtgtgc tgaagccatc gttgcggggc tgtctgtaga gaccctggag ggcacgacac
tggaggtggg ctgcagcggg gacatgctca ctatcaacgg gaaggcgatc atctccaata aagacatcct
agccaccaac ggggtgatcc actacattga tgagctactc atcccagact cagccaagac actatttgaa
ttggctgcag agtctgatgt gtccacagcc attgaccttt tcagacaagc cggcctcggc aatcatctct
ctggaagtga gcggttgacc ctcctggctc ccctgaattc tgtattcaaa gatggaaccc ctccaattga
tgcccataca aggaatttgc ttcggaacca cataattaaa gaccagctgg cctctaagta tctgtaccat
ggacagaccc tggaaactct gggcggcaaa aaactgagag tttttgttta tcgtaatagc ctctgcattg
agaacagctg catcgcggcc cacgacaaga gggggaggta cgggaccctg ttcacgatgg accgggtgct
gaccccccca atggggactg tcatggatgt cctgaaggga gacaatcgct ttagcatgct ggtagctgcc
atccagtctg caggactgac ggagaccctc aaccgggaag gagtctacac agtctttgct cccacaaatg
aagccttccg agccctgcca ccaagagaac ggagcagact cttgggagat gccaaggaac ttgccaacat
cctgaaatac cacattggtg atgaaatcct ggttagcgga ggcatcgggg ccctggtgcg gctaaagtct
ctccaaggtg acaagctgga agtcagcttg aaaaacaatg tggtgagtgt caacaaggag cctgttgccg
agcctgacat catggccaca aatggcgtgg tccatgtcat caccaatgtt ctgcagcctc cagccaacag
acctcaggaa agaggggatg aacttgcaga ctctgcgctt gagatcttca aacaagcatc agcgttttcc
agggcttccc agaggtctgt gcgactagcc cctgtctatc aaaagttatt agagaggatg aagcattagc
ttgaagcact acaggaggaa tgcaccacgg cagctctccg ccaatttctc tcagatttcc acagagactg
tttgaatgtt ttcaaaacca agtatcacac tttaatgtac atgggccgca ccataatgag atgtgagcct
tgtgcatgtg ggggaggagg gagagagatg tactttttaa atcatgttcc ccctaaacat ggctgttaac
ccactgcatg cagaaacttg gatgtcactg cctgacattc acttccagag aggacctatc ccaaatgtgg
aattgactgc ctatgccaag tccctggaaa aggagcttca gtattgtggg gctcataaaa catgaatcaa
gcaatccagc ctcatgggaa gtcctggcac agtttttgta aagcccttgc acagctggag aaatggcatc
attataagct atgagttgaa atgttctgtc aaatgtgtct cacatctaca cgtggcttgg aggcttttat
ggggccctgt ccaggtagaa aagaaatggt atgtagagct tagatttccc tattgtgaca gagccatggt
gtgtttgtaa taataaaacc aaagaaacat a

Human βIG-H3 Protein Sequence

MALFVRLLALALALALGPAATLAGPAKSPYQLVLQHSRLRGRQHGPNVACVQKVIGTNRKYFTNCKQWYQRKICGK
STVISYECCPGYEKVPGEKGCPAALPLSNLYETLGVVGSTTTQLYTDRTEKLRPEMEGPGSFTIFAPSNEAWASLP
AEVLDSLVSNVNIELLNALRYHMVGRRVLTDELKHGMTLTSMYQNSNIQIHHYPNGIVTVNCARLLKADHHATNGV
VHLIDKVISTITNNIQQIIEIEDTFETLRAAVAASGLNTMLEGNGQYTLAPTNEAFEKIPSETLNRILGDPEALRD
LLNNHILKSAMCAEAIVAGLSVETLEGTTLEVGCSGDMLTINGKAIISNKDILATNGVIHYIDELLIPDSAKTLFE
LAAESDVSTAIDLFRQAGLGNHLSGSERLTLLAPLNSVFKDGTPPIDAHTRNLLRNHIIKDQLASKYLYHGQTLET
LGGKKLRVFVYRNSLCIENSCIAAHDKRGRYGTLFTMDRVLTPPMGTVMDVLKGDNRFSMLVAAIQSAGLTETLNR
EGVYTVFAPTNEAFRALPPRERSRLLGDAKELANILKYHIGDEILVSGGIGALVRLKSLQGDKLEVSLKNNVVSVN
KEPVAEPDIMATNGVVHVITNVLQPPANRPQERGDELADSALEIFKQASAFSRASQRSVRLAPVYQKLLERMKH

TABLE II

Mouse βig-h3 Gene Sequence and Corresponding βIG-H3 Protein Sequence
Mouse βig-h3 Gene Sequence

ggcacgagcc tgctttcatc gtgggtccgc gcgtgctcca gctccatggc gctcctcatg cgactgctga
ccctcgctct ggcactgtct gtgggccccg ctgggaccct tgcaggtccc gccaagtcac cctaccagct
ggtgctgcag catagccggc tccggggtcg ccagcaeggc cccaatgtat gtgctgtgca gaaggtcatt
ggcaccaaca agaaatactt caccaactgc aagcagtggt accagaggaa gatctgcggc aagtcgacag
tcatcagtta tgagtgctgt cctggatatg aaaaggtccc aggagagaaa ggttgcccag cagctcttcc
gctctcaaat ctgtatgaga ccatgggagt tgtgggatcg accaccacac agctgtatac agaccgcaca
gaaaagctga ggcctgagat ggagggaccc ggaagcttca ccatctttgc tcctagcaat gaggcctggt
cttccttgcc tgcggaagtg ctggactccc tggtgagcaa cgtcaacatc gaactgctca atgctctccg
ctaccacatg gtggacaggc gggtcctgac cgatgagctc aagcacggca tgaccctcac ctccatgtac
cagaattcca acatccagat ccatcactat cccaatggga ttgtaactgt taactgtgcc cggctgctga
aggctgacca ccatgcgacc aacggcgtgg tgcatctcat tgacaaggtc atttccacca tcaccaacaa
catccagcag atcattgaaa tcgaggacac ctttgagaca cttcgggccg ccgtggctgc atcaggactc
aataccgtgc tggagggcga cggccagttc acactcttgg ccccaaccaa cgaggccttt gagaagatcc
ctgccgagac cttgaaccgc atcctgggtg acccagaggc actgagagac ctgctaaaca accacatcct
gaagtcagcc atgtgtgctg aggccattgt agctggaatg tccatggaga ccctgggggg caccacactg
gaggtgggct gcagtgggga caagctcacc atcaacggga aggctgtcat ctccaacaaa gacatcctgg
ccaccaacgg tgtcattcat ttcattgatg agctgcttat cccagattca gccaagacac tgcttgagct
ggctggggaa tctgacgtct ccactgccat tgacatcctc aaacaagctg gcctcgatac tcatctctct
gggaaagaac agttgacctt cctggccccc ctgaattctg tgttcaaaga tggtgtccct cgcatcgacg
cccagatgaa gactttgctt ctgaaccaca tggtcaaaga acagttggcc tccaagtatc tgtactctgg
acagacactg gacacgctgg gtggcaaaaa gctgcgagtc tttgtttatc gaaatagcct ctgcattgaa
aacagctgca ttgctgccca tgataagagg ggacggtttg ggaccctgtt caccatggac cggatgttga
cacccccaat ggggacagtt atggatgtcc tgaagggaga caatcgtttt agcatgctgg tggccgccat
ccagtctgca ggactcatgg agatcctcaa ccgggaaggg gtctacactg tttttgctcc caccaatgaa
gcgttccaag ccatgcctcc agaagaactg aacaaactct tggcaaatgc caaggaactt accaacatcc
tgaagtacca cattggtgat gaaatcctgg ttagcggagg catcggggcc ctggtgcggc tgaagtctct
ccaaggggac aaactggaag tcagctcgaa aaacaatgta gtgagtgtca ataaggagcc tgttgccgaa
accgacatca tggccacaaa cggtgtggtc tatgccatca acactgttct gcagccgcca gccaaccgac
cacaagaacg aggagatgag ctggcagact ctgcccttga aatcttcaaa caggcgtcag cgtattccag
ggctgcccag aggtctgtgc gacttgcccc tgtctatcag cggttactgg agaggatgaa gcattagcag
gaagaccgag gaggagagcc ctgcagcagc ttcccgccag tttctctcag tttgccaaag agaccattga
atgtttttga aaccaaagag cacacttcaa catacatggg cgcaccatat tgagatctga gccttggacg
ggtagggaag gggttaaggg gagaaaggtt ctttttagct ttgatccctc caaaccgtgg ttgttaaccc
attcgaatat acagatctgg cagtcatagc ttggcaccaa attcccgaaa gacctctcga aagcatgaat
ttcctgactg tgccaaggcc tgataaaggg aactacggca tcttggagct cacaaatgtg aatcaagcag
tccggcattc tggaaagcct tggcatggtt ctgtaaagct cttgtaccgc tggagaaacg gcatcactat
aagctatgag ttgaactgtt tctgtcaagt atgtcttgtg tccacacatg gtttggatgc ttctatattg
gccctgccca ggtagaaagg gtaagaagaa catgtagaat ccagattccc tgagtgtgag ggacccatgg
tgcatttgta ataa

Mouse βIG-H3 Protein Sequence

MALLMRLLTLALALSVGPAGTLACPAKSPYQLVLQHSRLRGRQHGPNVCAVQKVIGTNKKYFTNCKQWYQRKTCGK
STVISYECCPGYEKVPGEKGCPAALPLSNLYETMGVVGSTTTQLYTDRTEKLRPEMEGPGSFTIFAPSNEAWSSLP
AEVLDSLVSNVNIELLNALRYHMVDRRVLTDELKHGMTLTSMYQNSNIQIHHYPNGIVTVNCARLLKADHHATNGV
VHLIDKVISTITNNIQQIIEIEDTFETLRAAVAASGLNTVLEGDGQFTLLAPTNEAFEKIPAETLNRILGDPEALR
DLLNNHILKSAMCAEAIVAGMSMETLGGTTLEVGCSGDKLTINGKAVISKNDILATNGVIHFIDELLIPDSAKTLL
ELAGESDVSTAIDILKQAGLDTHLSGKEQLTFLAPLNSVFKDGVPRIDAQMKTLLLNHMVKEQLASKYLYSGQTLD
TLGGKKLRVFVYRNSLCIENSCIAAHDKRGRFGTLFTMDRMLTPPMGTVMDVLKGDNRFSMLVAAIQSAGLMEILN
REGVYTVFAPTNEAFQAMPPEELNKLLANAKELTNILKYHIGDEILVSGGIGALVRLKSLQGDKLEVSSKNNVVSV
NKEPVAETDIMATNGVVYAINTVLQPPANRPQERGDELADSALEIFKQASAYSRAAQRSVRLAPVYQRLLERMKH

TABLE III

Rabbit βig-h3 Gene Sequence and Corresponding βIG-H3 Protein Sequence
Rabbit βig-h3 Gene Sequence

atggcgctct tcgtgcggct gctggctctc gccctggctc tggcttgggc cccgccgcga ccctggccgg
ccccgccaag tctccctacc agctggtact ccagcatagc cggctccgcc gccagcagca cggccccaac
gtgtgcgctg tgcagaaggt catcggcacc aacaggaagt acttcaccaa ctgcaagcag tggtaccaga
ggaaaatctg tggcaaatca accgtcatca gctacgagtg ctgtcctggc tatgaaaagg tccccgggga
gagaagctgt ccagcagccc tcccactcgc caacctctac gagaccctgg gggttgttgg atcgaccacc
acccagctgt acacagaccg cacggagaaa ctgaggcctg agatggaggg gcccggccga ttcaccatct
tcgcccccag caacgaggcc tgggcttcct tgccagcgga ggtgctggac tccctggtga gcaacgtcaa
catcgagctg ctcaacgccc tgcgctacca catggtggac cgccgggtcc tcaccgacga gctgaagcac
ggcatggccc tcacctccat gtaccagaac tccaaattcc agatccacca ctatcccaac gggatcgtga
ccgtgaactg cgcccggctg ctgaaggccg accaccatgc caccaacggc gtggtgcacc tcatcgacaa
ggtcatctcc actgtcacca acaacatcca gcagatcatc gagatcgagg acacctttga gaccctgcgg
gctgccgtgg ccgcatcggg gctcaacacc ctgctcgaga gtgatggcca gttcacgctc ttggccccaa
ccaacgaggc caaagagaag atccctactg agactttgaa ccggatcttg ggtgatccag aggccctgag
agacctgctg aacaaccaca tcctgaagtc agccatgtgt gctgaagcca ttgtcgccgg gctgtccatg
gagaccctgg aggccaccac actggaggtg ggctgcagcg gggacatgct caccatcaac ggcaaggcca
tcatctccaa taaagacgtc ttggccacca acggtgtcat tcacttcatc gatgagctgc tcatccccga
ctccgccaag acgctgtctg agctggctgc aggatccgac gtctccacgg ccatcgacct tttcggacaa
gctggcctcg gcactcacct ctctggaaat gagcggctca ccctgctggc ccccctgaat tctgtgttcg
aagaaggagc ccctccaatt gatgcccata caaggaattt gcttcggaac cacataatta aagaccagct
ggcctctaag tatctgtacc atggacagac cctggacacg ctgggaggca aaaagctgag agtttttgtt
tatcgtaaca gcctgtgcat cgagaacagt tgcatcgctg cccatgacaa gagggggagg tacgggacgc
tgttcaccat ggaccggatg ctgacgcccc ccagtggcac cgtcatggac gtcttgaagg gggacaaccg
ctttagcatg ctggtggccg ccatccagtt ccgcaggctg actgagaccc tcaaccggga aggggcctac
actgtcttcg ctcccaccaa cgaagccttc caagccctgc caccaggaga gctgaacaaa ctgttgggaa
atgccaagga acttgccgac atcctgaaat accatgtggg cgaagaaatc ctggtgagcg ggggcatcgg
gaccctggtg cggctgaagt ccctccaggg cgacaagcta gaagtcagct cgaaaaacaa tgcggtgagt
gtcaacaagg agcctgttgc tgaaagtgac atcatggcca caaatggcgt ggtctatgcc atcaccagcg
ttctgcagcc tccagccaac agacctcagg aacgagggga tgaacttgca gactctgcgc ttgagatctt
caaacaagcg tcggcgtttt ccagggcttc ccagaggtct gtgcgactag cccctgtcta tcagaggcta
ttggaaagga tgaagcacta acgcagcaga ccacaggagg aaggcaccgt ggcagctgcc caccagcatc
tttgtttgcc aaagagactg ttttggaaac caaatatcac ccttcagtgt acatggcccg caccctaatg
agacctgagc ctggggcagt gggggcagga gggagagaag tctttatttt

Rabbit βIG-H3 Protein Sequence

GALRAAAGSRPGSGLGPAATLAGPAKSPYQLVLQHRSLRRQQHGPNCAVCQKVIGTNRKYFTNCKQWYQRKICGKS
TVISYECCPGYEKVPGERSCPAALPLANLYETLGVVGSTTTQLYTDRTEKLRPEMEGPGRFTIFAPSNEAWASLPA
EVLDSLVSNVNIELLNALRYHMVDRRVLTDELKHGMALTSMYQNSKFQIHHYPNGIVTVNCARLLKADHHATNGVV
HLIDKVISTVTNNIQQIIEIEDTFETLRAAVAASGLNTLLESDGQFTLLAPTNEAKEKIPTETLNRILGDPEALRD
LLNNHILKSAMCAEIVAGLSMETLEATTLEVGCSGDMLTINGKAIISNKDLVATNGVIHFIDELLIPDSAKTLSEL
AAGSDVSTAIDLFGQAGLGTHLSGNERLTLLAPLNSVFEEGAPPIDAHTRNLLRHNIIKDQLASKYLYHGQTLDTL
GGKKLRVFYRNSLCIENSCIAAHDKRGRYGTLFTMDRMLTPPSGTVMDVLKGDNRFSMLVAAIQFRRLTETLNREG
AYTVFAPTNEAFQALPPGELNKLLGNAKELADILKYHVGEEILVSGGIGTLVRLKSLQGDKELVSSKNNAVSVNKE
PVAESDIMATNGVVYAITSVLQPPANRPQERGDELADSALEIFKQASAFSRASQRSVRLAPVYQRLLERMKH

Mouse Models

General of βIG-H3-null Mutant Mice

βIG-H3 Knockout Mouse

Electroporation of engineered gene target vectors into embryonic mouse stem (ES) cells are utilized to generate βIG-H3-null mutant mouse embryos. In order to design a targeting vector to generate βIG-H3-null mice, the murine homologue of the human βig-h3 gene (Skonier et al., 1994) are isolated using PCR from adult mouse heart tissue. Murine and human βIG-H3 expression patterns at the mRNA level are similar (86%) and the sequences are highly related (90%) at the amino acid level (Skonier et al., 1994). Targeted inactivation of one of the βigh3 alleles are accomplished by replacement of selected exons with the neomycin resistance gene. Colonies surviving G418 and gancyclovir selection are analyzed by Southern-blot analysis for the presence of homologous recombination and whether transmission of the mutant allele followed normal Mendelian segregation ratios. Viable βIG-H3-null embryos are allowed to develop into homozygous mutant and heterozygous newborn pups which are examined for general overall health and gross developmental abnormalities compared to control littermates.

To determine if the targeting approach produced a null allele, tissue from homozygous mutants and heterozygous mice are evaluated and compared to wild type mice. Northern blot analysis are performed, using a probe against the full-length coding sequence for βig-h3 transcripts. Reverse transcription-PCR is used to reveal whether there are any other major or minor transcripts within skeletal muscle RNA resulting from the use of cryptic splicing sites in the neomycin cassette in homozygous mutants and heterozygous mice. Sequencing of the RT-PCR product will reveal mutations on the nucleotide level. Translation of any altered transcripts will produce a protein lacking the amino acid region encoded by exons 2, 4 and 12, including part of the original signal sequence. A polyclonal antibody generated against amino acids 71-683 of human βIG-H3 is used to indicate whether the mutant protein can be detected in βIG-H3-deficient skeletal tissues by immunoblot or immunofluorescence analysis.

Antisense Techniques Provide Long-Term Inhibition In vivo in Adult Animals

Antisense inhibition offers a different approach from gene knockout techniques because it is not used in embryos but in adult animals. This alternative approach for studying deleterious effects of βIG-H3 involves delivering antisense RNA by viral vector designed to give prolonged antisense effects in vivo. Specific antisense oligonucleotides to βig-h3 are created to inhibit βIG-H3 protein production in adult mice. To prolong the effect of antisense inhibition, DNA is inserted in the antisense direction in viral vectors. Recombinant adeno-associated virus provides a versatile system for gene expression studies and therapeutic applications, such as Phase I trials for gene therapy in cystic fibrosis currently ongoing (He et al., 1998). The adeno-associated virus transfers genes to a broad spectrum of cell types and does not require active cell division, like some other viral vectors (He et al., 1998). Although, viral vector delivery systems are disclosed, any DNA delivery system using anti-sense or regular DNA can be used including liposome carriers, or any other DNA delivery system.

This procedure is followed as described in He et al., 1998, where first murine βig-h3 is generated using PCR, the generated murine βig-h3 is then homologously recombination induced into the viral genome, and the combined genome is infected into adult mouse quadriceps via intramuscular injections. The engineered DNA is incorporated into the myoblast chromosomes, resulting in a loss of translation for βIG-H3. By using both methods of gene transfer (into developing embryos and adult mice), experiments test the structure and integrity of the muscle tissue and the MTJ in the mutant βIG-H3 mice.

Evaluation of βIG-H3 Knockouts

Physical Appearance Using Light Microscopy

βIG-H3-null mutant mice are first grossly examined for any overt signs of myopathy. To examine the structural effects at the cellular level in these mutant mice, hematoxylin and eosin stained frozen or paraffin-embedded sections of the quadricep femoris and the diaphragm muscles between the ages of 8 days and 9 months are evaluated. The examination looks for changes associated with muscular dystrophy, such as widely scattered clusters of necrotic myocytes or regenerating myocytes with internally placed nuclei. Clusters of necrotic myocytes generally increase in both number and size as the mice increase in age. In wild type mice, the numbers of centrally placed nuclei never exceeded 1%. Evaluations of five-hundred myocytes per muscle are taken. In addition to necrosis, regeneration, and central nucleation, βIG-H3-deficient muscle are also evaluated for a broad spectrum of other dystrophic changes. The most prominent of these include atrophy, hypertrophy, fiber splitting, and endomysial fibrosis. A qualitative comparison of fiber type distribution are assessed with ATPase staining, and staining characteristics with NADH and Gomori trichrome stains are evaluated as well. Light and confocal microscopy are used to analyze the MTJ and muscle morphology and staining results.

Sarcolemmal Integrity in βIG-H3-Deficient Muscle

To test whether mutation of the βIG-H3 gene leads to damage of the plasma membrane, βIG-H3-deficient embryos and adult mice are intravenously injected with Evans blue dye (EBD), a normally membrane-impermeant molecule. Evans blue (Sigma Chemical Co., St. Louis, Mo.) are dissolved in PBS (10 mg/ml) and sterilized by passage through membrane filters with a pore size of 0.2 μm. Mice are injected intravenously with 0.25 μm/10 g body weight of the dye solution through the tail vein. Animals are sacrificed 6 hours after injection by cervical dislocation. During the time period between injection and cervical dislocation, animals are kept in standard laboratory cages. All mice are skinned and inspected for dye uptake in the skeletal muscles, indicated by blue coloration. Evans Blue dye penetrates into the cytoplasm of fibers with compromised sarcolemmal integrity. An obvious uptake upon macroscopic inspection of the blue tracer into skeletal muscles of heterozygous mice versus control mice indicates a disruption in integrity. Fibers that take up the tracer typically will show pathologic plasma membrane permeability. Muscle sections for microscopic Evans blue detection are incubated in ice-cold acetone at −20° C. for 10 min, and after a rinse with PBS, sections are mounted with Vectashield mounting medium (Vector). Sections are observed under a MRC-600 laser scanning confocal microscope (Bio Rad Laboratories, Hercules, Calif.). Hematoxylin and eosin counterstaining are performed to examine whether characteristic features of degeneration and necrosis are also present.

Additionally, membrane damage in 7 to 10 week old βIG-H3-deficient mice and adult mice (depending on the method used) are evaluated by measuring the release of muscle specific pyruvate kinase (PK) into the circulating blood. Activities of muscle specific pyruvate kinase isozyme found in the blood serum are measured as previously documented (Edwards and Watts, 1981). Blood are collected from the retro-orbital sinus of the mice and the serum stored at −80° C. prior to measurements. Age-matched wild-type, heterozygous, and homozygous mice are all tested for normal serum levels of PK activity. Should homozygous mice exhibit high serum levels of PK activity (as demonstrated in the mdx mice model), this indicates that membrane damage has occurred.

βIG-H3 Expression in βIG-H3-null Mutant Mice

Immunofluorescence analysis are performed for βIG-H3 in βIG-H3-deficient mice. Polyclonal βIG-H3 antibody generated against bacterial fusion protein containing βIG-H3 residues 210-683 have been previously described(LeBaron et al., 1995; O'Brien et al., 1996; Rawe et al., 1997; Skonier et al., 1994) and we are preparing additional polyclonal anti-serum against full-length βIG-H3. Other components are also examined by immunofluorescence microscopy, including components of the dystrophin complex (DGC), laminin alpha-2 chain, and alpha and beta-dystroglycan. Expression of the newly identified E-sarcoglycan (Ettinger et al., 1997) and the 25 kD DGC component sarcospan (Crosbie et al., 1997) are also analyzed and compared to homozygous and wild-type control mice.

To further examine the expression of βIG-H3, immunoblot analysis are performed on isolated membrane preparations from control and homozygous mutant skeletal muscle. Muscle preparations are examined for their susceptibility to degradation by calcium dependent cysteine proteases, calpain I and II. Use of calpain I and II inhibitors will allow the preservation of protein integrity in the membrane preparations from βIG-H3-null mice. Coomassie Blue staining and staining for caveolin-3, the dihydropyridine (DHPR), and the ryanodine receptor can be used to show that equivalent levels of membrane proteins were present in control and homozygous mutant preparations. Western blot will confirm immunofluorescence analysis. Antibodies are obtained by conventional polyclonal serum production (rabbits) and through academic and commercial sources.

Restoration of βIG-H3 Function Using Gene Transfer Techniques

Adeno-Associated Viral Infection

The present invention is based on the hypothesis that βig-h3 gene replacement may correct primary mutations in individuals with LGMD Type 1A and possibly Duchennes MD (Congenital MD). Several mutations in βIG-H3 have been identified as contributing to 5q31-linked corneal dystrophies (Klintworth et al., 1998; Korvatska et al., 1999; Munier et al., 1997; Streeten et al., 1999), suggesting mutant βIG-H3 molecules may affect the natural organization of ECM and cells. Therefore, this invention suggests that βIG-H3 mutations in skeletal muscle may be a causative factor in Limb Girdle Type 1A MD, where mutations have been pinpointed to the 5q31 chromosome as well. Mutations can be identified through direct sequence analysis of genetic material, by sequence analysis of βIG-H3 cDNA generated by reverse transcriptase polymerase chain reaction (RT-PCR) of mRNA from donor patients and by probing for specific mutations. Hybridization analysis using nucleic acid probes can identify specific point mutations in the βig-h3 gene. Once βIG-H3 mutation are identified in LGMD type 1A patients, functional replacement gene are introduced to the effected tissue of the patient to ascertain their effectiveness in treating the disease. βIG-H3-null mice models are used to test this theory. The βig-h3 gene is deleted, the resulting mice are examined on structural, biochemical, and molecular levels, and then the βig-h3 gene is replaced to determine whether the effect is beneficial in restoring proper function. This can be accomplished through the use of an expression vector to deliver sequences that encode a functional βIG-H3 protein. An appropriate expression vector can safely and efficiently deliver exogenous nucleic acid to a recipient cell in the mouse. In order to achieve effective gene therapy, the expression vector used must be designed for efficient cell uptake and gene product expression, e.g. an adenovirus-based or adeno-associated virus (AAV) based gene delivery vector. An adeno-associated virus (AAV) based vector can also be used as a delivery system. Some examples are described by Carter et al., (1989) U.S. Pat. No. 4,797,368; Lebkowski et al. (1992) U.S. Pat. No. 5,153,414; *Srivastava et al., (1993) U.S. Pat. No. 5,252,479; Lebkowski et al. (1994) U.S. Pat. No. 5,354,678; *Wilson et al., (1998). AAV is an integrating DNA parvovirus, a naturally occurring defective virus that requires other viruses, such as adenovirus or herpes viruses as helper viruses (“Handbook of Parvoviruses”, ed., *P. Tijsser, CRC Press, (1990)). AAV vectors have been demonstrated functional in a wide variety of cell types, including differentiated and non-dividing cells, suggesting a potential for this vector system for successful in vivo gene delivery to muscle tissue.

In the preferred embodiment, a nucleotide sequence encoding the deficient βig-h3 gene are inserted into an adenovirus-based expression vector. Several genes have been successfully expressed using adenovirus based vectors including p53(Wills et al., 1994), dystrophin (Vincent et al., 1993), erythropoietin (Descamps et al., 1994), ornithine transcarbamylase (Stratford-Perricaudet et al., 1990), adenosine deaminase (Mitani et al., 1994), interleukin-2 (Haddada et al., 1993), and alpha-1-antitrypsin (Jaffe et al., 1992). The use of adenovirus based vectors in gene therapy is considered promising for a number of reasons, including the wide range of host cells, and the mechanism of expression from the adenoviral vector which occurs without chromosomal integration, eliminating the risk of insertional mutagenesis Gregory et al., (1997) U.S. Pat. No. 5,670,488; McClelland et al., (1998) U.S. Pat. No. 5,756,086; Armentano et al., (1998) U.S. Pat. No. 5,707,618; Saito et al., (1998) U.S. Pat. No. 5,731,172, herein incorporated by reference, describes several recently developed adenovirus-based expression vectors, and their use in gene therapy. Alternatively, a gutted adenovirus delivery system can be used (Clemens et al., 1996).

An AAV based vector can also be used as a delivery system. Some examples are described by Carter et al., (1989) U.S. Pat. No. 4,797,368; Lebkowski et al., (1992) U.S. Pat. No. 5,153,414; Srivastava et al., (1993) U.S. Pat. No. 5,252,479; Lebkowski et al., (1994) U.S. Pat. No. 5,354,678; Wilson et al., (1998) U.S. Pat. No. 5,756,283, herein incorporated by reference. AAV vectors have been demonstrated functional in a wide variety of cell types, including differentiated and non-dividing cells, suggesting a potential for this vector system for successful in vivo gene delivery to muscle tissue. Other possible gene expression systems for gene therapy include retroviral-based vectors and delivery systems (Miller, 1990) and also plasmid-based nucleic acid delivery systems (Eastman et al., (1998) U.S. Pat. No. 5,763,270, incorporated herein by reference).

In the preferred embodiment, a cytomegalovirus promoter element are used to drive gene expression from the expression vector. However, the expression vector can utilize a variety of regulatory sequences to achieve a therapeutic level of expression. Tissue specific regulatory sequences can also be used to restrict gene expression to a specific target tissue (Kuang et al., 1998). The method of delivery of the gene expression system to the target tissue varies with the expression system used. In preferred embodiments, upon diagnosis of the deficient βIG-H3 species, an appropriately packaged adenovirus-based expression vector construct are delivered via intramuscular injection of the βIG-H3-deficient mouse muscle tissue. Recipient tissue comprises muscle in the mouse that is, or is predicted to be, affected by the βIG-H3 gene deficiency. The mouse βIG-H3 cDNA sequence are subcloned into the pAdRSVpA adenovirus vector through standard methods of homologous recombination with Ad5 backbone d1309 by the University of Iowa Gene Transfer Vector Core. Lysates from the infected cells are collected and tested for the expression of βIG-H3 using a polyclonal antibody. Recombinant viruses are plaque purified 3×, amplified and concentrated using established methods (Davidson et al., 1994; Graham and van der Eb, 1973). Recombinant adenovirus injections are performed as previously described (Holt et al., 1998).

Administration of the gene to all deficient muscle tissues represents one therapeutic option. However, significant therapeutic benefits can also be achieved by selective administration to specific target muscles, to restore or prevent the loss of specific motor functions. For example, specific treatment of a small number of muscle groups can restore or prevent further deterioration of function, thereby enabling the patient to continue to eat without assistance. Administration of the deficient βig-h3 gene should optimally occur at as early a stage in disease progression as diagnosis permits, preferably, prior to the onset of severe muscle damage. Genetic diagnosis of the disease prior to the onset of the pathology allows gene therapy intervention at an extremely early stage in life.

To test the hypothesis that βig-h3 gene transfer could restore the normal function in mutated-βIG-H3-expressing myoblasts, direct plasmid DNA injections in the myoblasts are performed. Studies involving other proteins, such as dystrophin, have previously shown that de novo expression in a small percentage of fibers can be achieved by direct injection of plasmid DNA expression vectors in mdx mouse skeletal muscle.

Plasmid DNA Injection

Homozygous (−/−) mutant βIG-H3 mice are anesthetized by intraperitoneal injection of sodium pentobarbital (Nembutal, Abbott Laboratories) at a calculated dose of 75 mg/kg. The skin overlying the quadriceps femoris muscle are disinfected and a 1 cm vertical incision are made. Plasmid βIG-H3 DNA (100 μg) and 25 μg of β-galactosidase reporter plasmid DNA in a total volume of 100 μl normal saline (0.9% NaCl w/v) are injected into the quadriceps femoris muscle (Acsadi et al., 1991). The incision are closed with 3-4 sutures. Mice recover with continual supervision and were housed post-operatively at the UTSA Animal Care Facility. Seven days to 9 months post-injection, mice are sacrificed. Injected and uninjected quadriceps femoris muscle are removed by dissection, embedded in Tissue-Tek O.C.T. compound, and quickly frozen in liquid nitrogen. Serial sections are stained with antibodies to βIG-H3 and analyzed with confocal microscopy.

Analysis of β-galactosidase Activity

Serial cryosections are fixed in 0.5% glutaraldehyde in PBS for 15 min. at room temperature. After extensive washing with PBS, sections are treated with 1 mg/ml X-gal in β-galactosidase detection solution (20 mM K₂Fe(CN)₆, 20 mM K₂Fe(CN)₆×3H₂O, 2 mM mgCl₂ for 2-4 hours. Sections are counterstained with eosin, mounted with Permount, and viewed by light microscopy.

Cell Therapy

There is an alternative to gene therapy and this experimental plan are also attempted here. Myoblasts, grown in vitro, have been suggested to be effective in experimental “cell therapy” for hereditary muscle diseases (Gussoni et al., 1992). By fusing with mature or regenerating fibers of the host, implanted myoblasts could form hybrid myofibers thus contributing to the production of the normal gene product that was missing from the host (Rando and Blau, 1994). This has been applied to muscular dystrophies in mice (Partridge et al., 1989). The mdx mouse, which in the genetic homolog of the human form of Duchenne Muscular Dystrophy, has a defect in dystrophin (Sicinski et al., 1989). Transplantation of normal myoblasts into mdx muscle leads to expression in dystrophin in hybrid fibers, and also protects those fibers from the characteristic pathologic changes(Morgan et al., 1990). In the preferred embodiment, cells from a biopsy of diseased muscle tissue are propagated in culture under conditions appropriate for the formation of myotubes (Muroya et al., 1994). An expression vector containing βig-h3 cDNA are introduced to the muscle cells, and the recipient cells are examined, through either morphological or biochemical assays, for restoration of muscle tissue. Delivery of wild-type βIG-H3 should produce normal, functional cells. An adenovirus construct encoding human βIG-H3 are used to test the ability of exogenously provided βIG-H3 cDNA to restore the complex when the cells are transplanted into the host's skeletal muscle. An adenovirus construct encoding another gene, such as human delta-sarcoglycan, and an adenovirus construct without an insert, are used as controls. To circumvent a possible immune response against the neoantigen or adenovirus itself, the βIG-H3 adenovirus are injected into the quadriceps femoris of 2 day old βIG-H3-deficient pups. The recombinant adenovirus are directly injected into the muscle. Five days after injection, muscle tissue are analyzed for βIG-H3 expression by scanning confocal microscopy.

Distribution of BIG-H3 on the Ultrastructural Levels in Mouse Model Paradigms

Transmission Electron Microscopy (TEM)

To indicate any localization and structural differences in the sarcomere, biopsied tissue from wild-type, heterozygous, and βIG-H3-null homozygous mouse tissue are examined with TEM. Additionally, two other mouse models are also examined on the ultrastructural level, including the dystrophic, α7 integrin-deficient mouse (Mayer et al., 1997) and the well-characterized laminin α2 (merosin) chain-deficient mdx mouse model indicative of congenital muscular dystrophy (Vachon et al., 1997). These two models are examined because our preliminary evidence indicated that βIG-H3 binds the α7β1 integrin. These two models, involving an α7 mutant and the known ligand for the α7β1 integrin (merosin), may provide additional information concerning βIG-H3 in muscular dystrophies. There is currently no murine model for Limb Girdle MD type 1A. The main feature of the MTJs in α7-deficient mice is the loss of the interdigitations and the accumulation of necrotic material accompanied by retraction of the myofilaments from the plasma membrane (Miosge et al., 1999). This is significant since skeletal muscle cells bind βIG-H3 through the α7β1 integrin in vitro. Because these animal models have common protein and genetic defects similar to those seen in people with muscular dystrophies, they have been widely used to examine the effectiveness of gene therapy. The tissue are rinsed quickly in Sorenson's buffer (230 milliosmoles, pH 7.4) and fixed in 4% paraformaldehyde overnight. The tissue is washed, blocked in a 4:1 ratio of methanol to hydrogen peroxide, and blocked in BSA for twelve hours prior to incubation overnight with anti-βIG-H3 or normal rabbit IgG. Next, the tissue sections are washed and incubated overnight at 4° C. with a second goat anti-rabbit antibody conjugated to HRP. Antibodies are localized with DAB serving as the chromagen. Sections are washed in Sorenson's buffer, fixed in 1.0% osmium tetroxide, and dehydrated in ethyl alcohol and propylene oxide. Sections are then embedded in Embed 812 and polymerized for 48 hours at 60° C. 1-2 μM-thick sections are cut and stained with toluidine blue to determine location using optical microscopy. Once established, thin sections of 80-100 nm are cut on a Reichert Jung Ultracut E and examined for antibody tissue localization using a JEOL 1230 TEM. Sections are stained with saturated uranyl acetate and Reynold's lead citrate. Digital images are collected with a Gatan Dual View camera.

LGMD Biopsied Tissue

BIG-H3 Expression and Distribution in LGMD Biopsied Tissue Versus Age-matched, Non-affected Tissue

Immunohistochemistry Using Light and Confocal Microscopy

Tissue biopsies obtained from LGMD-affected and non-affected patients are gathered and tested for βIG-H3 localization using antibodies generated against βIG-H3. For anti-βIG-H3 immunohistochemistry, tissue are formalin-fixed, embedded in paraffin, sectioned, and baked onto microscope slides. The sections are re-hydrated and treated with 0.1% trypsin. To block endogenous peroxidase activity, tissue are incubated twenty minutes with a 4:1 ratio of methanol and hydrogen peroxide. Finally, the sections are incubated with 1% BSA for one hour at ambient temperature. Anti-βIG-H3 antibody in a 1% BSA/PBS buffer are applied for an overnight incubation. Additionally, in order to demonstrate specificity of the anti-βIG-H3 antibody, competition experiments are performed on similar sections. For this, anti-βIG-H3 antibody are pre-absorbed with purified recombinant bIG-H3 prior to the application to tissue sections.

The tissue sections are washed and incubated with a secondary goat anti-rabbit antibody conjugated to horseradish peroxidase (HRP) or fluorescein (FITC). Normal rabbit antibody serves as a control on identically-treated tissue sections. Antibodies are localized with DAB as recommended by the manufacturer. Sections are counterstained for 5 min. each in hematoxylin and then eosin, dehydrated with ethanol and xylenes, mounted with Permount, and examined by light microscopy. Those sections stained with immunofluorescence procedures are analyzed with a Bio-Rad MRC-600 laser scanning confocal microscope.

Transmission Electron Microscopy (TEM)

To indicate any differences in the sarcomere or ultrastructure of the diseased tissue, the biopsied sample are rinsed quickly in Sorenson's buffer (230 milliosmoles, pH 7.4) and fixed in 4% paraformaldehyde overnight. The tissue is washed, blocked in a 4:1 ratio of methanol to hydrogen peroxide, and blocked in BSA for twelve hours prior to incubation overnight with anti-βIG-H3 or normal rabbit IgG. Next, the tissue sections are washed and incubated overnight at 4° C. with a second goat anti-rabbit antibody conjugated to HRP. Antibodies are localized with DAB serving as the chromagen. Sections are washed in Sorenson's buffer, fixed in 1.0% osmium tetroxide, and dehydrated in ethyl alcohol and propylene oxide. Sections are then embedded in Embed 812 and polymerized for 48 hours at 60° C. 1-2 μM-thick sections are cut and stained with toluidine blue to determine location using optical microscopy. Once established, thin sections of 80-100 nm are cut on a Reichert Jung Ultracut E and examined for antibody tissue localization using a JEOL 1230 TEM. Sections are stained with saturated uranyl acetate and Reynold's lead citrate. Digital images are collected with a Gatan Dual View camera.

Immunocytology and Histology

For control purposes, F59 antibody are used alongside the βIG-H3 antibody usage. F59 antibody recognizes multiple fast myosin heavy chain (MyHC) isoforms in both embryonic skeletal muscle tissue (Crow and Stockdale, 1986) and primary myoblast cultures (Karsch-Mizrachi et al., 1989). Additionally, Masson's trichrome stain are used to chemically authenticate muscle and tendon. This stain is based on a previously published procedure modified (Lillie, 1940). Tissue sections are placed in Harris's hematoxylin for twenty seconds, rinsed in de-ionized water, and stained in scarlet-acid fuchsin. After rinsing in de-ionized water, sections are placed in a phosphomolybdic acid solution to prepare the sections for placement in aniline blue stain. Tissue sections are rinsed in 1.0% acetic acid and de-ionized water, dehydrated, cleared to xylene, and mounted in Permount.

BIG-H3 Mutated in LGMD Patients

Sequence Analysis

Blood cells from healthy human donors and patients exhibiting LGMD type 1A, as well as other LGMDs and Congenital MD are tested for βIG-H3 mutations. This are accomplished through nucleotide sequence analysis and comparing the DNA from the affected and non-affected patients. Total RNA are extracted with Trizol reagent as suggested by the manufacturer and cDNA are isolated using RT-PCR and oligo-dT nucleotide probes. Nucleotide sequences are determined by the dideoxy chain termination method (Sanger et al., 1977). This information provides the basis for the deduced amino acid sequence. Particular importance are emphasized on whether known amino acid mutations observed in corneal dystrophies (Munier et al., 1997) are present in the biopsied tissue, as well as possible novel mutations. Examination of the βIG-H3 sequence also includes whether the known Arg-Gly-Asp (RGD) tripeptide and the glycosaminoglycan-binding sequences are fully present (Attached manuscripts, and whether the H1 and H2 sequences within the Fasiclin-I-like repeats are conserved (Kawamoto et al., 1998).

Western Blot Analysis

For this assay, the amount of muscle obtained by biopsy should be sufficient to enable the extraction of βIG-H3 in a quantity sufficient for analysis. Diseased and non-affected age-matched biopsied tissue are homogenized by mechanical disruption using apparatus such as a hand operator or motor driven glass homogenizer, a Waring blad blender homogenizer, or an ultrasonic probe. Homogenization are carried out in EDTA-extraction buffer (10 mM EDTA, 50 mM Tris/HCl, pH 7.5, 150 mM NaCl, 1 mM PMSF, 0.75 mM benazamidine, 1 mg/ml aprotinin, 1 mg/ml leupeptide, 1 mg/ml pepstatin A) on ice for 2 hrs. Following centrifugation, extracellular matrix solubilized in this manner can then be processed by conventional methods in western blotting analytical formats. The proteins are first separated on a 3-12% SDS polyacrylamide gel (Laemmli, 1970) followed by transfer to a solid support. These proteins are transferred by western blot technique to an Immobilon P transfer membrane. The detection of the transferred protein components can be accomplished by the use of general protein dyes such as Amido black or Coomassie brilliant blue. Antibodies which are specific for βIG-H3 are incubated with the membrane. The specific binding of these antibodies to the muscle tissue sample is detected through the use of labeled secondary antibodies by conventional techniques. In short, the membrane are then washed in PBS and a goat anti-rabbit-horseradish peroxidase antibody is applied. The membrane is washed in PBS and DAB used as the chromagen. Identical fractions are run on SDS-PAGE and stained with Coomassie blue and compared to those on the immunoblot. Comparison between diseased and non-diseased tissue are evaluated in terms of molecular size and the formation of dimers or spliced variants of βIG-H3. Differences in βIG-H3 function may also be due to altered glycosylation of the protein. Glycosylation of βIG-H3 in normal and diseased tissues are assessed by conventional immunoblot for glycosylation detection. Oxidation of sugar are introduced and free aldehyde groups forming a complex with biotin. The complex are detected by application of streptavidin and alkaline phosphatase and NBT/BCIP.

βIG-H3 Mutations and Affects on Sarcomere Stability and Necrosis of Muscle Fibers

Transmission Electron Microscopy (TEM)

To provide evidence of possible disruption in structural differences in the sarcomere, biopsied LGMD tissue are compared to age-matched control tissue on the ultrastructural level. The tissue are rinsed quickly in Sorenson's buffer (230 milliosmoles, pH 7.4) and fixed in 4% paraformaldehyde overnight. The tissue is washed, blocked in a 4:1 ratio of methanol to hydrogen peroxide, and blocked in BSA for twelve hours prior to incubation overnight with anti-βIG-H3 or normal rabbit IgG. Next, the tissue sections are washed and incubated overnight at 4° C. with a second goat anti-rabbit antibody conjugated to HRP. Antibodies are localized with DAB serving as the chromagen. Sections are washed in Sorenson's buffer, fixed in 1.0% osmium tetroxide, and dehydrated in ethyl alcohol and propylene oxide. Sections are then embedded in Embed 812 and polymerized for 48 hours at 60° C. 1-2 μM-thick sections are cut and stained with toluidine blue to determine location using optical microscopy. Once established, thin sections of 80-100 nm are cut on a Reichert Jung Ultracut E and examined for antibody tissue localization using a JEOL 1230 TEM. Sections are stained with saturated uranyl acetate and Reynold's lead citrate. Digital images are collected with a Gatan Dual View camera.

Mutated βIG-H3 in Functional Assays

This last section of our five-year experimental plan involves βIG-H3 mutations and how these mutations might affect the normal function of skeletal muscle. To do this, several functional assays are performed in vitro. The first step, however, is to isolate myoblasts with mutated βIG-H3 in culture. Three different primary myoblast lines are to be isolated. First, myoblasts are isolated from the genetically-altered βIG-H3-null murine models. Secondly, myoblasts are isolated from LGMD type 1A and Congenital MD patient muscle biopsies and thirdly, mutations found in the biopsied are generated, diseased tissue (from nucleotide sequencing) and transfect and propagate new myoblast cell lines.

Myoblast Isolation and Culture Methods

Primary Myoblast Isolation from βigh3-knockout Mice

Primary myoblasts are isolated from the quadricep muscle from homozygous (−/−) βIG-H3-knockout mice, heterozygous (−/+) βIG-H3-knockout mice, and wild-type mice. Skeletal muscle are dissected from both hindlimb quadricep muscles, trypsinized for ten minutes at 37° C., and seeded onto a substratum comprised of 50 μg/ml type I collagen. Chick embryo extract (300 μl/10 ml), insulin (10 ng/ml), and Basic Fibroblast Growth Factor (20 ng/ml) are added daily to F10 growth media plus 10% horse serum to maintain pure myoblast populations and to prevent differentiation (Clegg et al., 1987). Serial passages will eliminate contaminating fibroblast and fat cell populations. The purity of primary cell cultures are established by immunohistochemical detection of the myosin fast chain using monoclonal anti-myosin (F59) antibody (Karsch-Mizrachi et al., 1989). Serum levels are decreased to 1% to induce myotube formation. Primary skeletal muscle cells are maintained at 37° C. in 95% ambient air and 5% CO₂.

Primary Myoblast Isolation from Biopsied Tissue

The amount of muscle obtained by biopsy from LGMD type 1A and Congenital MD-affected and age-matched, non-affected patients should be sufficient to isolate myoblast populations in a quantity sufficient for in vitro culture. Although βIG-H3 mutations in type 1A LGMD is our primary goal, the procedures may also be used to examine the potential that mutated βIG-H3 contributes toward development of other MD types. The skeletal muscle biopsy are trypsinized in trypsin-EDTA for ten minutes at 37° C. and seeded onto a substratum comprised of 50 μg/ml type I collagen. Cells from a biopsy of diseased muscle tissue are propagated in culture under conditions appropriate for the formation of myotubes. Chick embryo extract (300 μl/10 ml), insulin (10 ng/ml), and Basic Fibroblast Growth Factor (20 ng/ml) are added daily to F10 growth media plus 10% horse serum to maintain pure myoblast populations and to prevent differentiation until a myoblast population is established (Clegg et al., 1987). Serial passages will eliminate contaminating fibroblast and fat cell populations. The purity of primary cell cultures are established by immunohistochemical detection of the myosin fast chain using monoclonal anti-myosin (F59) antibody (Karsch-Mizrachi et al., 1989). Serum levels are decreased to 1% to induce myotube formation. Primary skeletal muscle cells are maintained at 37° C. in 95% ambient air and 5% CO₂. Primary Myoblast Transfection with βigh3 Mutations

For many of our experiments, the mutated form of βIG-H3 are tested to confirm their presence in muscular dystrophy patients. Therefore, the mutations are produced on the molecular level with site-directed mutagenesis and transfect mutated cDNA into a myoblast cell line C2C12 murine myoblast cells (ATCC number CRL-1772) (Yaffe and Saxel, 1977), utilizing conventional amplifiable expression vectors, thus potentially conferring a dominant negative phenotype to the cells. This method is preferred because in vitro systems can be used to study mutated βIG-H3. C2C12 cells are maintained in DMEM supplemented with 1.5 g/l of sodium bicarbonate and 10% heat-treated FBS. When testing functionality in myotubes, the differentiation of myoblasts to myotubes are accomplished by the replacement of 10% FBS with 2% horse serum, as previously described (Bennett and Tonks, 1997).

Full-length βIG-H3 from skeletal muscle cell extracts are generated with RT-PCR using the primer set of 5′ TGCCCGTCGGTCGCAAGCTTGC 3′ and 5′ TGTAGTGCTTCAAGCTTATGC 3′. Site-directed mutagenesis are performed as described by manufacturer s specifications using Promega's GeneEditor in vitro Site Directed Mutagenesis System. The mutated strand of βIG-H3 are synthesized using T4 DNA polymerase and T4 DNA ligase. The bacteria strain BMH71-18 mutS are transformed with βIG-H3, incorporating a mutagenesis reaction using heat shock methodology. BMH71-18 mutS cells containing plasmids with βIG-H3 mutations are selected with antibiotics and plasmids isolated with Wizard Prep DNA Plasmid Purification columns (Promega). The plasmid DNA are excised and ligated into PGEM-T vector (Promega), transfected into the bacterial strain JM109 and amplified. Those colonies containing the plasmid with the mutated βIG-H3 are selected for ampicillin resistance.

The plasmid DNA containing the mutated βIG-H3 are excised with the restriction enzymes and ligated into an appropriate the mammalian expression vector. A reporter gene, β-galactosidase, are co-transfected to ensure into which cells the vector and mutant βIG-H3 are successfully transfected. Plasmids with no insert as well as plasmids containing normal βIG-H3 are used as controls. To transfect C2C12 cells, reagents must be highly pure. Therefore, the Wizard PureFection Plasmid DNA Purification System from Promega is used to purify the plasmid DNA. With the highly pure plasmid DNA obtained from this system, a high-efficiency transfection using calcium phosphate-DNA precipitate formed in N,N-bis (2-hydroxyethyl)-2-amino-ethanesulfonic acid (BES) buffer, pH 6.95 are conducted. In short, plates of confluent myoblast cultures are incubated overnight while a calcium phosphate-DNA complex forms gradually in the medium under at atmosphere of 3% CO₂. With this method, approximately 50% of the cells on a plate stably integrate and express the transfected DNA. Transfected cells are tested for altered cell-to-cell contact and formation of myotubes.

Alternatively, the βIG-H3 and control plasmids are transfected into Chinese Hamster Ovary cells and the resultant recombinant products purified as described (attached manuscripts) and tested in cell adhesion experiments as described (attached manuscripts). An additional option is to utilize expression vectors that incorporate a polyhistidine tag (6× His) on the carboxyl terminus of the recombinant protein, thus possibly expediting the initial results of the effects that mutated βIG-H3 conveys to muscle biology. Because the plasmid contains the 6× His tag, the mutated βIG-H3 protein are easily isolated using a nickel resin column.

Western Blot Analysis

To determine whether all three isolated myoblast populations (see preceding section) express βIG-H3 in vitro, western blot analysis are performed. To do so, cell populations are homogenized separately by mechanical disruption using a hand-operated glass homogenizer. Homogenization are carried out in EDTA-extraction buffer (10 mM EDTA, 50 mM Tris/HCl, pH 7.5, 150 mM NaCl, 1 mM PMSF, 0.75 mM benazamidine, 1 mg/ml aprotinin, 1 mg/ml leupeptide, 1 mg/ml pepstatin A) and the extract are sit on ice for 2 hrs. Following centrifugation, extracellular matrix solubilized in this manner can then be processed by conventional methods in western blotting analytical format. The proteins are first separated on a 3-12% SDS polyacrylamide gel (Laemmli, 1970) followed by transfer to a solid support. These proteins are transferred by western blot technique to an Immobilon P transfer membrane. The detetection of the transferred protein components can be accomplished by the use of general protein dyes such as Amido black or Coomassie brilliant blue. Antibodies that are specific for βIG-H3 (Skonier et al., 1994) are incubated with the membrane overnight. The specific binding of these antibodies to the muscle tissue sample is detected through the use of labeled secondary antibodies by conventional techniques. In short, the blotted membrane are then washed in PBS and a goat anti-rabbit-horseradish peroxidase antibody applied. The membrane is washed in PBS and DAB used as the chromagen. Comparison between the myoblast populations are evaluated in terms of molecular size of βIG-H3 and the formation of dimers or spliced variants of βIG-H3.

βIG-H3 Expression in Isolated Myoblasts Upregulated by Growth Factors

Tendon fibroblasts have been shown to express transcripts for Transforming Growth Factor-β1 (TGF-β1), and TGF-β1 has the potential to act as a paracrine or autocrine factor in vivo (Heine et al., 1987). We previously showed that the C2C12 myoblast cell line secretes βIG-H3 and that this secretion appears to be responsive to a treatment of 20 ng/ml of TGF-β1 in vitro. Control cultures (no TGF-β1 treatment) were incubated under similar conditions and did not yield an increase in βIG-H3 expression.

TGF-β1 is one known factor that is known to modulate βIG-H3 expression in skeletal muscle cells. The effects of other selective individual growth factors on βIG-H3 expression in human muscle cells are tested in a similar experimental manner. The growth factors to be tested are described in Table IV.

TABLE IV
List of Growth Factors

| Growth Factor | Function |
|---|---|
| EGF (epidermal | active transport, DNA, RNA and protein, |
| growth factor) | synthesis, synergizes with IGF-1 and TGF-β |
| Basic Fibroblast | mitogen for many mesodermal cells |
| Growth Factor | |
| IGF (insulin-like | glucose uptake and oxidation; |
| growth factor) | amino acid uptake |
| Platelet-Derived | mitogen for mesodermal cells, wound repair, |
| Growth Factor | synergizes with EGF and IGF-1 |
| Dexamethasome | non-physiological and soluble |
| | inducer of differentiation |

All three populations of isolated myoblasts (see first section in C) are incubated at 37° C. for 24 hrs., conditioned media collected and concentrated using a 30,000 molecular weight cut-off (MWCO) filter. Concentrated media loaded on polyacrylamide gels are normalized to total protein. Samples of conditioned media are run on a sodium-dodecyl sulfate polyacrylamide gel (SDS-PAGE) and transferred by western blot technique to an Immobilon P transfer membrane. The respective membranes are incubated with βIG-H3 antibody overnight at 4° C., washed in PBS, and a goat anti-rabbit-horseradish peroxidase antibody applied for two hours. Normal rabbit IgG will serve as a negative control. The membrane are washed in PBS and DAB used as the chromagen. Identical fractions are run on SDS-PAGE and stained with Coomassie Blue to visualize protein transfer.

Cell Types Found at the MTJ Contribute to the Secretion of βIG-H3

Whether βIG-H3 is synthesized by myoblast termini exclusively, or perhaps also made by cells within the perichondrium and tendon is not clear. These tissues adjacent to the muscle fibers may contain cells that synthesize βIG-H3 protein and subsequently associating the protein with the skeletal muscle cells. Precedence for this organization has been documented where type IV collagen synthesized by fibroblasts contributes to the developing basal lamina of myotubes (Kuhl et al., 1984). To test whether βIG-H3 is produced by myogenic cells or tendon fibroblasts, an experimental design that are used is an in vitro co-culture model. Firstly, separate cultures of isolated murine tendon fibroblasts and skeletal muscle myoblasts are maintained. Both cultures are first tested for βIG-H3 expression by western blot analysis. Proteins are separated in a Bio-Rad Mini-protean II Dual Slab gel system in a running buffer consisting of IM Tris, glycine, and SDS (pH 8.3) and electrophoretically transferred (Towbin et al., 1979) to an Immobilon P membrane. Molecular weight protein markers are visualized with Coomassie Blue stain. Membranes are dried completely prior to the addition of anti-βIG-H3-ig antibody in 1% BSA/PBS and incubated overnight at 4° C. After three washes with PBS, anti-rabbit antibody conjugated to horseradish peroxidase is applied as a secondary antibody and incubated with the membrane for two hours at 4° C. The membrane is washed in PBS and developed using DAB as the chromagen.

Co-Cultures of Myoblasts and Tendon Fibroblasts

Co-cultures are then established by seeding both cell types in one culture dish on a substratum of type I collagen. Cultures of fibroblasts only, skeletal muscle cells only and co-cultures of both cell types are allowed to grow until fusion can be seen between myoblasts. The cells are fixed and stained overnight with βIG-H3 antibody. Determination of βIG-H3 staining on the surface of myotubes in each condition are performed with phase and confocal microscopy and quantified by testing samples of conditioned medium normalized to protein on SDS PAGE as described above. Additionally, conditioned medium from tendon fibroblasts are added to primary cultures of muscle cells to observe whether the identical result can be obtained.

Macromolecular Complexes at the MTJ

βIG-H3 Mutations and Binding to Molecules of the ECM

Evidence suggests βIG-H3 can bind to ECM molecules, including collagens. βIG-H3 binds collagen type I (attached manuscripts; Hashimoto, 1997), collagen types II and IV (Hashimoto et al., 1997) and co-isolated with type VI (Rawe et al., 1997). During murine embryogenesis, collagen expression patterns, including types I, II, and III are similar to βIG-H3 expression pattern (Cheah et al., 1991), appearing in all chondrogenic tissue and in tendon morphogenesis in the late embryonic stages. The similarities between collagen expression patterns and βIG-H3 are striking and may indicate an in vivo interaction. Types I, II, and III collagen are major components of tendon (Birk and Mayne, 1997; Kosher et al., 1986; Trotter et al., 1983; Williams et al., 1980), and collagen fibrils emanate from tendon, into adjacent muscle fibers, possibly playing a role in the structural attachment of tendon to skeletal muscle fibers (Trelstad and Birk, 1984).

Transmission Electron Microscopy (TEM)

When thin sections of mouse MTJ were examined utilizing immuno-TEM and anti-βIG-H3 antibody, the results revealed that βIG-H3 appears to associate with a meshwork of extracellular fibrils within the space between cells. These fibers were similar in morphology and striations as collagens. βIG-H3 also localized along the myoblast cell membrane. The overall ultrastructural assessment is consistent with the observation that anti-βIG-H3 antibody localization coincided distinctively with digit-like extensions of skeletal muscle that protrude into the adjacent perichondrium, suggesting a specific binding function of this protein to collagens and at myotube termini. Using TEM (previously described) and other ECM antibodies, proteins that are involved with βIG-H3 at the MTJ can be determined. Antibodies tested will include those produced against collagens localized to MTJs, fibronectin, laminin and proteoglycans.

Enzyme-Linked ImmuAssays (ELISA)

Additionally, it is noted that most, if not all, extracellular matrix adhesion proteins bind to glycosaminoglycan. Cell surface-associated heparan sulfate proteoglycans have been shown to serve as receptors for ECM proteins, sometimes working in conjunction with integrins at cell-substratum contacts (LeBaron et al., 1988; Woods et al., 1986). Our studies examined βIG-H3 for heparin-binding consensus sequences. Such sequences are proposed to pattern X-B-B-X-B-X, where B is a basic amino acid and X is a hydropathic amino acid (Cardin and Weintraub, 1989). The B-B-X-B pattern was utilized with MacVector version 6.5 sequence analysis software (Oxford Molecular Group, Madison, Wis.) to search for putative heparin binding sequences in beta-ig in the cDNA deduced amino acid sequence of human βIG-H3. Two separate sequences that met the B-B-X-B criteria were revealed, suggesting βIG-H3 might exhibit an affinity for glycosaminoglycans. Heparan sulfate proteoglycans have been localized to the developing myotendinous junction (Trotter et al., 1983). Thus the potential for βIG-H3 to bind heparin in ELISA assays and column chromatography are examined as well.

Like heparan sulfate proteoglycans and collagens, fibronectin also localizes to the developing myotendinous junction as part of the ECM surrounding myofibers (Tidball, 1984; Trotter et al., 1983). Biochemical data in our studies indicates βIG-H3 may have an affinity for fibronectin. Fibronectin typically forms complexes with other ECM proteins, including collagens and heparan sulfates (Mosher, 1989). Taken together, these comprehensive results suggest βIG-H3 has the potential to form an adhesive macromolecular complex by interactions with other ECM proteins, including collagens, fibronectin, and glycosaminoglycans, at the developing MTJ.

The methods of the present invention can also be practiced in an enzyme-linked immunoadsorbent assay (ELISA) format. Many variations of this assay exist as described in Voller, A., Bidwell, D. E. and Bartlett, A., The Enzyme Linked Immunoadsorbent Assay (ELISA): A guide with abstracts of microplate applications, Dynatech Laboratories, Alexandria, Va. (1979). ECM molecules, including normal βIG-H3, mutated βIG-H3, fibronectin, laminin, proteoglycans and glycosaminoglycans, and collagens, are immobilized onto microtiter wells by drying overnight. After re-hydrating substrata with phosphate buffered saline, all wells are blocked with BSA for one hour. βIG-H3 are then added to each substratum and incubated at 37° C. for two hours. Antibodies to βIG-H3 are added to each well and incubated at 4° C. overnight. Wells that are coated with βIG-H3 are re-hydrated, blocked with BSA, incubated with anti-βIG-H3 antibody overnight and serves as a control for the antibody. All wells are washed with PBS and a secondary antibody conjugated to alkaline-phosphatase are applied. The reaction are quantified using the substrate PNPP. A yellow color change absorbed at 405 nm is indicative of a positive reaction (i.e. binding of βIG-H3 occurred to the specific substratum).

Statistical Analysis

To determine statistical significance for ELISA assays, results from experiments±SD are evaluated and significance calculated by paired Student's t-tests. Differences for statistical tests are considered significant when p≦0.05.

βIG-H3, a Mechanical Link Between Muscle Fibers and Tendon Collagen Fibers

CytoDetacher Technique

The strength of muscle cell adhesion to βIG-H3 is tested by using a cytodetachment technique previously used for other cell types (Athanasiou et al., 1999). The cytodetacher applies a detaching force parallel to the base of a single muscle cell on a specific substratum, in this case, normal versus mutated βIG-H3. Results are tested against control substrata of laminin, type I collagen, and fibronectin.

This method uses cantilever beam theory to measure such a small force. This technique has been utilized in various studies for sensing microforces, especially in areas of skeletal and heart muscle physiology and cell adhesion. Force is calculated by determining the amount of beam deflection that occurs while detaching the cell. The beam is driven and controlled by a linear, computer-controlled, piezoelectric translator. A data acquisition board (NB-MIO-16XL-42, National Instrument) and the LabVIEW object-oriented programming language (National Instrument) on a Macintosh computer are used to collect data and control the piezoelectric translators.

Mechanical Loading of Myoblasts and Increased βIG-H3 Expression

Mechanical Loading Model

Mechanical stimuli can cause changes in muscle structure that indicates that mechanisms exist for transducing mechanical stimuli into signals that influence gene expression (Tidball et al., 1999). MTJs are highly specialized sites of force transmission across the muscle cell membrane and are responsive to changes in their mechanical environment. MTJs show adaptations to modified muscle loading which suggest that these are transcription ally distinct domains in muscle fibers that may experience local regulation of expression of structural proteins (Tidball et al., 1999).

To study this aspect of βIG-H3, an in vitro mechanical loading model are used as previously described (Tidball et al., 1999). Cells have been shown to upregulate their attachment strength to ECM ligands if increased tension is applied through their integrins (Choquet et al., 1997), indicating that cells can rapidly reinforce cytoskeletal linkages locally at applied force application sites. In short, C2C12 muscle cells are subjected to cyclic strains using a mechanical cell stimulator. This involves cell stretching at a 6.7% deformation of the membrane in 20-second cycles for 24 hours. Cultures grown under identical conditions but not subjected to loading will serve as a control.

Northern Blot Analysis

Myoblasts subjected to load or no load treatments are lysed with extraction buffer (150 mM NaCl, 25 mM Tris-HCl (pH 7.4), 2.0% (w/v) Triton-X-100, 1 mM phenylmethylsulfonyl fluoride (PMSF), 10 mg/ml leupeptin, and 2 mg/ml bovine serum albumin (BSA)(Fraction V, 98% pure). The extract are pelleted by centrifugation. Total RNA are extracted with Trizol reagent according to manufacturer specifications. 30 μg of total RNA are run on a 1.25% agarose gel containing 5% formaldehyde and transferred to Hybond N membrane (Amersham). RNA are cross-linked to the membrane using a Stratagene UV crosslinker. Membranes are then prehybridized and hybridized using standard methods. Washes are carried out at 65° C. in 1× SSC/1% SDS initially, then 0.1%.SSC/0.1% SDS. Blots are exposed for autoradiography. First strand cDNA are synthesized from total mouse heart RNA using oligo-dT primers. RT-PCR is accomplished utilizing sense (5′-CGAACTGCTCAATGCTCTCCGC-3′) and antisense (5′-CCCCGATGCCTCCGCT AACC-3′) primer sequences. Probes for βIG-H3 transcripts corresponded to nucleotides 540-1798 of mouse βIG-H3 cDNA (Skonier et al., 1994) and are used to analyze the 1259-bp amplified product.

Big-h3 Mutations and Myoblast Binding and Spreading

Determination of Purified βIG-H3 Concentration

To determine the initial concentration of purified βIG-H3 from our culture population of mutated βIG-H3-transfected myoblasts, protein concentrations are determined using a bicinchoninic acid (BCA) assay based on Smith et al., 1985. BCA is a stable and highly specific reagent for Cu+. Amino acid side chains in a protein react with Cu+2, producing Cu+ in an alkaline environment. Two molecules of BCA bind one cuprous ion, yielding a purplish color, measured at 562 nm. βIG-H3-containing samples are compared against standard albumin protein curves.

Solid Phase Attachment Assays

We previously demonstrated that normal βIG-H3 supports skeletal muscle cell attachment and spreading. Mutated βIG-H3 are tested to determine their adhesive substratum properties for skeletal muscle cells. Recombinant normal and mutated βIG-H3 are immobilized by coating microtiter wells and allowing the protein to dry. BSA and type I collagen will serve as control substrata. Wells are subsequently washed with PBS. Prior to experiments, all substrata are blocked with BSA in PBS. Skeletal muscle cells are cultured and used in solid phase attachment assays. To reduce the chance that endogenous proteins synthesized by these cells would affect adhesion, cells are pre-incubated with the protein inhibitor cycloheximide (CHX) in serum-free media for one hour. Concentration curves of CHX-treatment on myoblasts are generated to determine the optimal concentration of CHX application. CHX are included throughout the adhesion experiments. After CHX treatment, cells are detached using 1.0 mM EDTA, washed, seeded at a density of 40,000 cells per well in their respective serum-free medium and substratum and incubated at 37° C. in a 5% CO₂ humidified atmosphere for one hour. Unattached cells are rinsed from the substratum with PBS (three washes per well), and attached cells quantified by addition of WST-1 reagent and recording adsorption at 450 nm. WST, a tetrazolium salt, is cleaved by mitochondrial dehydrogenases and is an efficient way to measure cell viability and quantitate cell number. The formazan dye produced by viable cells is quantified by a spectrophotometer by measuring the absorbency of the dye in the well. Trypan blue exclusion (Brus and Glass, 1973) will indicate the viability of CHX-treated cells on βIG-H3.

Cell Spreading

Myoblasts are treated with optimal concentrations of CHX for one hour and passed onto wells coated with βIG-H3 or fibronectin. Phase-contrast microscopy interfaced with software and image capture hardware obtained by Media Cybernetics (Silver Springs, Md.) are used, including a mouse drawing device. Computer images of attached and spread cells are randomly captured in a double blind test and traced with the mouse drawing device. Software calibrated to the microscope will calculate the area per traced cell. Graphical representation will indicate the breadth of cells spread on a fibronectin versus a normal βIG-H3 or mutated βIG-H3 substrata after a one-hour time period.

Statistical Analysis

To determine statistical significance for cell adhesion assays, results from experiments±SD are evaluated and significance calculated by paired Student's t-tests. Differences for statistical tests are considered significant when p≦0.05.

Actin Distribution in Myoblasts Adhered to Normal and Mutated βIG-H3

Very little is known about the cytoplasmic architecture of a cell in response to βIG-H3. Therefore, the relative distribution of individual cytoskeletal components and their organization within the skeletal muscle cell are examined using immunofluorescence and confocal microscopy. A comparative analysis of cells plated on a fibronectin, laminin, normal βIG-H3, or mutated βIG-H3 substrata are examined. In light of the cytoskeletal role in mechanotransduction, phalloidin staining results may reveal actin filaments in a strategic position to mediate physical deformation of the plasma membrane. Indeed, a mechanism similar to this has been previously documented, suggesting that when magnetic force was applied to fibroblasts, an increase bundling of cortical actin enhanced the mechanotransduction stabilization of the cell membrane and overall increased membrane rigidity (Glogauer et al., 1998). Fluorescent phallotoxins can be used to label ƒ-actin in cultured cells (Faulstich et al., 1988). To determine if skeletal muscle cells that attach to a substratum comprised of mutated or normal βIG-H3 form an organized actin cytoskeleton after one hour, rhodamine-phalloidin are used to visualize any stress fiber formation.

Cells are pre-incubated with an optimal concentration of cycloheximide for one hour, detached with 1.0 mM EDTA from sub-confluent cultures and seeded onto tissue-culture chamber slides to which a βIG-H3, laminin, or fibronectin substratum has been adsorbed by drying overnight. After a one-hour attachment period at 37° C., unattached cells are washed off with PBS and adherent myoblasts fixed and permeabilized in a 1:1 acetone/methanol solution at room temperature. Cells are blocked with bovine standard albumin. Rhodamine-phalloidin stock is diluted (12.5 μL/1 mL) in a 1% BSA solution and added to the fixed cells for 30 minutes at 37° C. Cells are washed with PBS and mounted with Mounting Medium for Fluorescence. Confocal microscopy are utilized to determine actin structure.

Integrin Receptor and Recognition of βIG-H3 in Muscle Cells

We previously demonstrated some evidence that the α7β1 integrin is responsible for myoblast binding to βIG-H3 (attached manuscripts). The α7β1 cell surface receptor localizes almost exclusively to the MTJ starting at E14, and the α7 subunit is enriched at the MTJ in the adult (Bao et al., 1993). Indeed, α7β1 appears to be an essential link to ensure muscle integrity, particularly in regions subject to mechanical stress (Yao et al., 1997). This experiment was conducted using inhibitory antibodies to particular integrin subunits in an in vitro system. Other cell types, however, have been shown to use other integrins to recognize βIG-H3 (Kim et al., 2000). To address the issue of α7β1 binding to βIG-H3 in myoblasts, northern blot analysis is first used to determine which integrins are expressed by myoblasts in culture.

Northern Blot Analysis

Cultured myoblasts are lysed with extraction buffer (150 mM NaCl, 25 mM Tris-HCl (pH 7.4), 2.0% (w/v) Triton-X-100, 1 mM phenylmethylsulfonyl fluoride (PMSF), 10 mg/ml leupeptin, and 2 mg/ml bovine serum albumin (BSA)(Fraction V, 98% pure). The extract are pelleted by centrifugation. Total RNA are extracted with Trizol reagent according to manufacturer specifications and 30 μg of total RNA are run on a 1.25% agarose gel containing 5% formaldehyde and transferred to Hybond N membrane (Amersham). RNA are cross-linked to the membrane using a Stratagene UV crosslinker. Membranes are then prehybridized and hybridized using standard methods. Washes are carried out at 65° C. in 1× SSC/1% SDS initially, then 0.1%.SSC/0.1% SDS. Blots are exposed for autoradiography. First strand cDNA probes are synthesized from total mouse heart RNA using oligo-dT primers.

PCR are used to detect integrins, including those for the spliced variants for α7 and β1 subunits. The α7 extracellular segment appears to be spliced at exons, termed X1 or X2, located between a variable amino acid sequence region. Sites neighboring this region have been implicated in maintaining active receptor conformation and ligand specificity and affinity (Ziober et al., 1997; Ziober et al., 1993). Mouse C2C12 skeletal myoblasts and embryonic skeletal muscle cells express equal levels of X1 and X2 with only a slight increase in X2 in differentiated C2C12 myotubes.

Splice variants have also been observed in the cytoplasmic domain of the α7 subunit, designated as the α7A and α7B forms (Ziober et al., 1997; Ziober et al., 1993). Increases in the α7A form were documented during mouse embryogenesis and into adulthood. This suggests that α7A may regulate differentiation, and associated events such as ligand binding, cytoskeletal interactions, and signal transduction (Hogervorst et al., 1993). Different cytoplasmic splice variants could be important in regulating the quality and strength of signal input from the extracellular space (Ziober et al., 1993).

Like the variants seen in the α7 subunit, there are also four different isoforms for the β1 subunit: β1A, β1B, β1C, and β1D (Belkin et al., 1996). It is the cytoplasmic domain of the β1 subunit that is primarily required for interaction with the cytoskeleton. The β1D isoform has been identified as a muscle-specific splice variant of the β1 integrin subunit and has been shown to reinforce linkages made between the cytoskeleton and the ECM (Belkin et al., 1997). In skeletal muscle, β1D is concentrated at myotendinous junctions and associates with the α7A and α7B integrin subunit isoforms in the adult skeletal muscle. Modulation of the β1 integrin adhesive function by alternative splicing serves as a physiological mechanism, reinforcing the cytoskeleton-matrix link in muscle cells. This reflects the major role for the β1D integrin in muscle, where extremely stable association is required for contraction (Belkin et al., 1997). Overall, the presence of four α7 and four β1 integrin subunit isoforms indicate possible differences in integrin-ligand recognition, as well as differences in the myoblast cellular response and subsequent changes in intracellular signaling pathways.

Affinity Column Chromatography

Once the type of integrins expressed by muscle cells is established and the expression of α7β1 confirmed, myoblasts are cultured to sub-confluent levels. Cell extracts are obtained by adding extraction buffer (150 mM NaCl, 25 mM Tris-HCl (pH 7.4), 2.0% (w/v) Triton-X-100, 1 mM phenylmethylsulfonyl fluoride (PMSF), 10 mg/ml leupeptin, and 2 mg/ml bovine serum albumin (BSA, Fraction V, 98% pure) to the cultures, scraping with a rubber policeman, and pelleting the extract with centrifugation. The extract are equilibrated in the proper starting buffer and used immediately for column chromatography.

Normal and mutated βIG-H3 affinity columns are generated using purified recombinant βIG-H3. Separately, the proteins are coupled to Bio-Rad Affigel 10 beads at 1 mg protein/ml beads by incubation with rotation, for four hours, at 4° C. The column are equilibrated with the appropriate buffer, and the cell extracts are passed over the column. Unbound material are collected and the column washed. The bound material are eluted with a wash of 10 mM EDTA. Column eluates and cell extracts are electrophoresed in 10% sodium-dodecyl sulfate polyacrylamide gels. Samples are transferred by western blot technique to an Immobilon P transfer membrane. The membrane is incubated with 05-α7, a monoclonal antibody reactive with the extracellular domain of the α7 subunit (Song et al., 1992), and 014-β1 antibody, a monoclonal antibody reactive with β1 subunit (Song et al., 1992). The membrane are then washed in PBS and a goat anti-rabbit-horseradish peroxidase antibody is applied. The membrane is washed in PBS and DAB used as the chromagen. Identical fractions are run on SDS-PAGE and stained with Coomassie Blue. This experiment should provide information that indicates whether a) α7β1 binds normal βIG-H3 under these biochemical conditions and b) whether α7β1 has the ability to bind mutated βIG-H3.

α7β1 Form and Recognition of βIG-H3

Our previous results suggest that βIG-H3 binds skeletal muscle cells via the α7β1 integrin (attached manuscripts). If both normal and mutated forms of βIG-H3 indicate binding to α7β1 in the experiment described in the previous section, next a determination of which of the spliced variants of integrin subunits α7 and β1 are the ones to recognize βIG-H3 is made. To address this question, inhibitory antibodies are generated against the various spliced integrin isoforms in standard inhibition assays in vitro.

Inhibition Assays Using Function-Blocking Anti-Integrin Antibodies

Function-blocking anti-integrin antibodies are used to test for possible βIG-H3 and integrin interactions as previously described (Yao et al., 1997). Myoblasts are pre-incubated with CHX in serum-free media (DMEM) for one hour prior to experiments and CHX included throughout inhibition experiments. Inhibitory antibodies generated against different integrin subunits are used in the assays. Integrin subunits tested will include most of those found in developing skeletal muscle, including the α1 (Duband et al., 1992), α5 (Blaschuk and Holland, 1994), α6 (Bronner-Fraser et al., 1992), as well as the different spliced isoforms of the α7 (Bao et al., 1993; Burkin and Kaufman, 1999; Yao et al., 1997), and β1 (Menko and Boettiger, 1987) integrin subunits.

Cells are detached with 1.0 mM EDTA and incubated in suspension with anti-integrin antibodies for 45 minutes prior to seeding onto a βIG-H3 substratum made fom a solution containing 10 μg βIG-H3/mL PBS. Cells are also seeded onto a substratum comprised of 10 μg/ml laminin to demonstrate the effectiveness of the inhibition of the α7 and β1 subunit antibodies. After a one-hour time period, unattached cells are rinsed off, and WST-1 reagent applied. Absorbance recordings at 450 nm are taken after two hours and cell number attachment quantified.

Occupational Preference by the α7β1 Integrin for βIG-H3 or Laminin

Laminin is a major component of the ECM and is believed to play a prominent role in promoting myoblast adhesion, migration, proliferation, and differentiation (Ocalan et al., 1988). The α7β1 integrin is the predominant, if not only, laminin-binding integrin on skeletal muscle cells (Song et al., 1993). Integrins can bind several ligands, and generally, one ligand is recognized by several integrin heterodimers (De Arcangelis and Georges-Labouesse, 2000). Indeed, (α7β1, known primarily as a laminin binding receptor (Yao et al., 1997), has also been documented to bind and modulate other ECM protein binding, including the RGD sequence in fibronectin and the L-14 lectin (Gu et al., 1994).

Affinity Column Chromatography and Immunoblot Analysis

To test the possibility of selective modulation of the α7β1 interaction with βIG-H3 or laminin, primary murine myoblasts are first cultured. Primary myoblasts are obtained by dissecting skeletal muscle from tendon, trypsinize for ten minutes at 37° C., and then seed onto a substratum comprised of 50 μg/ml type I collagen. Chick embryo extract (300 μl/10 ml), insulin (10 ng/ml), and bFGF (20 ng/ml) are added daily to F10 growth media plus 10% horse serum to maintain pure myoblast populations and to prevent differentiation (Clegg et al., 1987). Serial passages will eliminate contaminating fibroblast and fat cell populations. The purity of primary cell cultures are established by immunohistochemical detection of the myosin fast chain using monoclonal anti-myosin (F59) antibody (Karsch-Mizrachi et al., 1989). Cells are maintained at 37° C. in 95% ambient air and 5% CO₂. Once cultures are established, cell extracts are obtained by adding extraction buffer (150 mM NaCl, 25 mM Tris-HCl (pH 7.4), 2.0% (w/v) Triton-X-100, 1 mM phenylmethylsulfonyl fluoride (PMSF), 10 mg/ml leupeptin, and 2 mg/ml bovine serum albumin (BSA)(Fraction V, 98% pure)) to the cultures, scraping with a rubber policeman, and pelleting the extract with centrifugation. The extract are equilibrated in the proper starting buffer and used immediately for column chromatography.

Laminin columns are generated using Englebreth-Holm-Swarm mouse tumor laminin. The protein are coupled to Bio-Rad Affigel 10 beads at 1 mg protein/ml beads by incubation with rotation, for four hours, at 4° C. The column are equilibrated with the appropriate buffer, and the cell extracts are passed over the column. Unbound material are collected and the column washed. The bound material are eluted with a wash of 10 mM EDTA. Column eluates and cell extracts are electrophoresed in 10% sodium-dodecyl sulfate polyacrylamide gels.

Samples are transferred by western blot technique to an Immobilon P transfer membrane. The membrane is incubated with 05-α7, a monoclonal antibody reactive with the extracellular domain of the α7 subunit (Song et al., 1992), and 014-β1 antibody, a monoclonal antibody reactive with β1 subunit (Song et al., 1992). The membrane are then washed in PBS and a goat anti-rabbit-horseradish peroxidase antibody is applied. The membrane is washed in PBS and DAB used as the chromagen. Identical fractions are run on SDS-PAGE and stained with Coomassie blue.

Since the α7β1 integrin should bind laminin, the influence of βIG-H3 on this particular ligand-receptor interaction is tested. Cell extracts are pre-incubated with purified normal or mutated forms of βIG-H3, passed over the laminin column, and eluted fractions are run on western blots as described above to determine whether α7β1 has an occupational preference for βIG-H3 over laminin and whether mutated βIG-H3 interferes with this preference.

PKC Activation in Myoblasts on Normal And/or Mutated βIG-H3

Inhibition Assays

Protein Kinase C (PKC) appears to be one of the key intermediates in integrin-mediated signaling in many cell types. In certain cell types, inhibition of PKC activity results in the inhibition of cell attachment and spreading as well as FAK phosphorylation. Myoblasts on fibronectin use α5 integrin for attachment and this integrin activates the PKC pathway (Disatnik and Rando, 1999). Myoblasts on fibronectin have been shown to biochemically activate FAK (Disatnik and Rando, 1999). The pathway for the α7β1 integrin is currently unknown. To test whether myoblasts on normal recombinant βIG-H3 or mutated βIG-H3 activate the PKC pathway, myoblast cultures are treated with the PKC inhibitor Calphostin C. Myoblasts are first allowed to adhere to normal and mutated βIG-H3 and then tested with a range of 0-1.0 μM Calphostin C. Fibroblasts on fibronectin are used as a control substrata. Myoblasts on laminin are also performed for comparison. Cells that round up with the treatment are suggestive of PKC pathway activation while on βIG-H3. Trypan blue dye are used to ensure cells are viable.

Kinases Activated or Inactivated in Muscle Cells Exposed to Soluble βIG-H3

Myoblasts (quantitated with a hemocytometer) are cultured on plastic until they reach sub-confluency. For comparison, myoblasts are also cultured on ECM substrata including fibronectin, laminin, type I collagen, and normal βIG-H3. Myoblasts are then treated with various concentrations of normal or mutated βIG-H3 in the soluble form. Myoblasts that have been maintained and not subjected to any βIG-H3 treatment serve as a control plate. After a three-hour incubation with soluble mutated or normal βIG-H3, myoblast cultures are lysed with extraction buffer (150 mM NaCl, 25 mM Tris-HCl (pH 7.4), 2.0% (w/v) Triton-X-100, 1 mM phenylmethylsulfonyl fluoride (PMSF), 10 mg/ml leupeptin, and 2 mg/ml bovine serum albumin (BSA, Fraction V, 98% pure) and the extract centrifuged. Samples are subject to electrophoresis in 10% sodium-dodecyl sulfate polyacrylamide gels and transferred by western blot technique to an Immobilon P transfer membrane. Memebranes are probed for serine, threonine, and tyrosine-activated kinases. Membranes are then washed in PBS and a goat anti-rabbit-horseradish peroxidase antibody is applied. Membranes are washed in PBS and DAB used as the chromagen. Identical fractions are run on SDS-PAGE and stained with Coomassie blue to visualize protein transfer and quantities.

βIG-H3 Mutation and Myoblast Migration

To test whether migrating myoblasts have a preference for soluble laminin or βIG-H3, Boyden Chamber Assays are used, assays performed in a modified blind-well apparatus. This assay indicates the preference of myoblasts to migrate to soluble βIG-H3 and laminin. For comparison, cell migration to mutated βIG-H3 is tested. To do this, myoblasts are first cultured as previously described. Next, serum-free medium supplemented with either normal or mutated βIG-H3, laminin, or fetal bovine serum (FBS) are added to the bottom wells of the chambers. Membranes coated with type I collagen (50 μg/ml) are set on top of the chambers and the myoblasts are seeded onto these membranes. This setup will provide us with chemotaxis information, revealing whether normal or mutated βIG-H3 or laminin, or both, have a chemotactic effect on myoblasts. FBS serves as a positive control since it contains numerous growth factors that initiate chemotactic behavior.

Additionally, testing is performed on immobilized, normal and mutated βIG-H3 and laminin for the ability to support myoblast migration. Membranes are prepared with all three substrata. Type I collagen and BSA will serve as control substrata on the migration membranes. One side of a porous membrane are coated with substratum and placed over the bottom wells of chamber apparatus, which are filled with growth medium plus fetal bovine serum (FBS). Myoblasts are seeded onto the membranes, allowed to incubate at 37° C. for two hours, and then unattached myoblasts removed from the membrane. The membranes are fixed in ethanol, stained with Coomassie Brilliant Blue, and the attached cell number to the underside of the membranes quantitated with light microscopy.

Statistical Analysis

To determine statistical significance for migration assays, results from experiments±SD are evaluated and significance calculated by paired Student's t-tests. Differences for statistical tests are considered significant when p≦0.05.

βIG-H3 and its Affect on Cell Growth

Growth Curve Assays

Growth curves are documented by starting with a known number of myoblasts in a culture dish. Normal and mutant βIG-H3 are added daily to the myoblasts in culture and control wells treated identically but without the βIG-H3 supplement. Myoblasts are allowed to proliferate for one week or before they differentiate into myotubes (become 100% confluent) and the cell number quantified with WST, a tetrazolium salt cleaved by mitochondrial dehydrogenases. This is an efficient way to measure cell viability and quantify cell number. The formazan dye produced by viable cells is quantified by a spectrophotometer by measuring the absorbency of the dye at 450 nm in the well. Trypan blue exclusion (Brus and Glass, 1973) will indicate the viability cells after one week.

Statistical Analysis

To determine statistical significance, results from experiments±SD are evaluated and significance calculated by paired Student's t-tests. Differences for statistical tests are considered significant when p≦0.05.

Big-h3 and its Affect on Cell Differentiation and Fusion

Cell Fusion and Differentiation Assays

Starting with C2C12 murine myoblast cells in a culture dish, cell fusion studies are performed. Normal and mutant βIG-H3 are added to a known number of myoblasts in culture. Cells will proliferate until they are 70% confluent at which time the medium (DMEM supplemented with 1.5 g/l of sodium bicarbonate and 10% heat-treated FBS) are replaced with differentiation medium as previously described (Bennett and Tonks, 1997) and soluble normal or mutant βIG-H3 added to myoblasts. After three days, myotubes fusion are observed and documented by light microscopy and digital image capture technology. Cells are fixed with 100% acetone, blocked in BSA/PBS, and incubated with βIG-H3 antibody. After several PBS washes, a second antibody conjugated to rhodamine is added, incubated with cells and then any unbound second antibody washed from wells. Antibody localization are observed using confocal microscopy and findings documented by recording digital images.

βIG-H3 and Nerve Process Movement

Preliminary studies using conditioned media from CHO cells expressing βIG-H3 indicated that immbolized βIG-H3 may support axon elongation in pheochromocytoma (PC12) cells. Further testing of this possibility is described here, using purified, recombinant βIG-H3 as an immobilized substrate. Rat PC12 cells (Drubin et al., 1985) are cultured in Optimem medium +5.0% FBS+100 ng/ml NGF on 30 μg/ml type I collagen-coated plates for one week prior to experiments.

In addition to PC12 cells, murine trigeminal ganglia and sciatic nerve, a peripheral nerve, are isolated and also tested for neurite extension on βIG-H3. In short, young, male Sprague-Dawley rats (days 30-32) are decapitated. The brain are removed from the skull, exposing the underlying trigeminal ganglia. The trigeminal ganglia from each hemisphere is dissected out from beneath the ventral skull bone and minced with a sterile scalpel. The ganglia is treated with 0.1% collagenase in DMEM for 1.5 hours at 37° C. Collagenase was removed by centrifugation, and the ganglia treated with 1× trypsin-EDTA for 30 minutes at 37° C. The tissue is dissociated by repeated passage through a 19-gauge needle and the trypsin inactivated with FBS. The cells are spun down, trypsin and FBS removed, and cells washed three more times with plain DMEM minus FBS. The final cell suspension is in DMEM+N1 supplement (insulin, transferrin, biotin, sodium selenite, putrescine, and progesterone)+glutamine.

Dissociated trigeminal neurons and PC12 cells are seeded onto βIG-H3, type I collagen, or BSA. βIG-H3 (30 μg/ml) and type I collagen (30 μg/ml) substrata are prepared by drying overnight in tissue culture-treated wells. The substrata are re-hydrated for fifteen minutes with PBS and unbound sites on the wells blocked with 10% BSA for two hours. Cells seeded onto these substrata are cultured overnight at 37° C. in 5% CO₂. 100 ng/ml of NGF is added to the serum-free cultures to promote extension. Any neurite outgrowth after 24 hours are stained with Coomassie Brilliant Blue. Neurite extension is assessed with light phase microscopy. Additional experiments including testing soluble βIG-H3 for its influence on neurite outgrowth and extension.

### EXPERIMENTAL SECTION

### βIG-H3 Expression During Mouse Embryogenesis

βIG-H3 is an extracellular matrix protein that in vitro binds to collagens and particular cell types. The protein sequence contains fasciclin-1 like repeats and peptide sequences suggesting that βIG-H3 may bind glycosaminoglycans and to members of the integrin family of cell adhesion molecules, suggesting βIG-H3 may play a role in developmental processes. This possibility was investigated by documentation of the spatiotemporal distribution of βIG-H3 during murine development. In situ hybridization of mouse embryos (E12.5-E18.5) indicated a prominent, distinct expression pattern for βIG-H3 message in connective tissue. βIG-H3 transcripts were abundantly expressed during mesenchymal cell condensation in areas of axial, craniofacial and appendicular primordial cartilage from E12.5-E14.5. Beginning at E15.5, βIG-H3 transcripts appeared in collagen-rich tissues, including dura mater and corneal stroma. During E16.5-E18.5, βIG-H3 transcripts were observed in proliferating chondrocytes and areas of endochondral ossification in joint and articular cartilage formation. In limited locations, βIG-H3 transcripts were detected in the nervous system and within associated tissues. The caudal region of the trigeminal ganglia at E14.5, part of the optic nerve sheath from E14.5-E18.5 and the endosteal dura from E14.5-E18.5 expressed βIG-H3. Connective tissues expressed βIG-H3 transcripts within the nasal septum and surrounding cartilage primordia, and in the pericardium, optic cup, kidney, ovary, esophagus, diaphragm, bronchi, trachea, corneal epithelium and during cardiac valve formation. The bladder, testes and regions near or within vibrissae had moderate levels of expression from E14.5-E17.5. The patterns of expression within connective tissue indicate that βIG-H3 is potentially involved in tissue morphogenesis. Cells derived from mesenchyme attached and spread onto a substratum comprised of purified recombinant βIG-H3. Taken together, the results indicate that βIG-H3 is expressed principally in collagen-rich tissues and interacts with molecules of the ECM and with cells in a manner that promotes cell attachment and tissue modeling in order to facilitate cartilage, bone and organ morphogenesis.

Introduction

Development of multicellular organisms is dependent on numerous and varied contacts of extracellular matrix (ECM) molecules and cells (Blaschuk, 1994). The ECM is comprised of collagens, proteoglycans, non-collagenous glycoproteins such as fibronectin, laminin, tenascin and likely yet-to-be discovered molecules. As new ECM molecules are investigated, information regarding their spatiotemporal expression is anticipated to provide a better understanding of their physiological function. Fairly recently, a gene responsive to transforming growth factor-β (TGF-β) was discovered by differential screening of an adenocarcinoma cDNA library ENRfu (Skonier et al., 1992). The newly identified gene, named Transforming Growth Factor-β Induced Gene-Human Clone 3 (βig-h3), encodes a 683 amino acid secretory protein that was designated βIG-H3 ENRfu (Skonier et al., 1992). βIG-H3 contains repeating units similar to recurring sequences found in fasciclin-I, a nerve cell growth cone guidance molecule expressed in developing Drosophila ENRfu (Zinn et al., 1988). Consensus sequences predicted to bind sulfated glycosaminoglycan ENRfu (Cardin and Weintraub, 1989) were discovered near the central portion of βIG-H3 and are potentially functional as βIG-H3 binds heparin-agarose (unpublished observation). Possibly mediating attachment to members of the integrin superfamily of cell surface adhesion receptors are the sequences Arg-Gly-Asp ENRfu (Pierschbacher and Ruoslahti, 1984), Asn-Lys-Asp-Ilu-Leu and Glu-Pro-Asp-Ilu-Met ENRfu (Kim et al., 2000b). Additionally, βIG-H3 binds collagens in vitro ENRfu (Hashimoto et al., 1997).

Immunochemistry and protein sequence analyses detected βIG-H3 in skin ENRfu (LeBaron et al., 1995), cornea ENRfu (Escribano et al., 1994; Hirano et al., 1996), bladder smooth muscle ENRfu (Billings et al., 2000) and as a component of elastic fibers ENRfu (Gibson et al., 1996). The distribution of βIG-H3 in adult tissues and the findings that βIG-H3 promotes cell adhesion ENRfu (Kim et al., 2000b; LeBaron et al., 1995) and binds to collagens ENRfu (Hashimoto et al., 1997; Rawe et al., 1997) and heparin suggests that βIG-H3 functions in development and tissue modeling, interacting with cells and ECM molecules. The βIG-H3 gene maps to human chromosome 5q31, a region proposed to contain genes that when mutated, then may play a pathogenic role, contributing toward the development of tumors and corneal and muscular dystrophies (see discussion). However, the normal physiologic function of βIG-H3 and mechanisms that may mediate its possible role in pathogenicities in vivo are not clear. To better understand the biology of βIG-H3, a developmental study was performed, anticipating that developmental processes occurring concurrently with changes in expression patterns of βIG-H3 help to reveal its physiological functions.

The current study provides new information addressing the spatiotemporal expression of βIG-H3 during development. A βIG-H3 specific RNA probe was synthesized to test for mRNA expression during various developmental stages of mouse embryos. Expression of βIG-H3 transcript was evident in all dense connective tissue, various epithelial and muscle tissue, and in chondrogenic tissue destined for cartilage or bone morphogenesis. In addition, mammalian expressed recombinant βIG-H3 promoted adhesion of cells derived from mesenchyme, but not cells derived from epithelium. The results of this study identify putative functions of βIG-H3 in mammalian embryonic processes in vivo. The expression pattern is consistent with studies reporting that in vitro βIG-H3 plays a role in cell adhesion and binds to ECM molecules.

Materials and Methods

Materials

The transcription vector pGEM-T and T7/SP6 RNA Polymerase Riboprobe reagents were from Promega Corporation (Madison, Wis.) and ³⁵S-rUTP was obtained from NEN Life Science Products Inc. (Boston, Mass.). Nylon membranes and oligo-dT primers were purchased from Boehringer-Mannheim (Indianopolis, Ind.). Photographic film and NBT-2 photographic emulsion was from Eastman Kodak (Rochester, N.Y.). Trypsin-EDTA was purchased from Mediatech, Inc. (Herndon, Va.). Glasgow's Minimum Essential Medium (GMEM) was from ICN Biochemicals (Costa Mesa, Calif.) and α-Minimum Essential Medium (MEM), Dulbecco's Modified Eagle Medium (DMEM), Ham's F-12, Trizol LS and antibiotics were purchased from Gibco BRL Life Technologies (Grand Island, N.Y.). Fetal bovine serum (FBS) was from Irvine Scientific (Santa Ana, Calif.). Cell proliferation reagent 4-[3-(4-iodophenyl)-2-(4-nitrophenyl)-2H-5-tetrazolio]-1,3-benzene disulfonate (WST-1) was obtained from Roche Molecular Biochemicals (Indianopolis, Ind.). YM membranes were from Amicon, Inc. (Beverly, Mass.). Superfrost Plus pre-coated microscope slides and glass coverslips were obtained from VWR Scientific Products (Sugarland, Tex.). Methionine sulfoximine (MSX), bovine serum albumin (BSA), yeast tRNA, triethanolamine, cycloheximide, 3′,3′-diaminobenzidine tetrahydrochloride (DAB), heparin-agarose, and all other reagent grade chemicals were from Sigma Chemical Company (St. Louis, Mo.).

Methods

Preparation of Embryonic Tissue for In situ Hybridization

Mouse embryos, days E12.5-E18.5, were fixed in 10% neutral formalin overnight at ambient temperature and processed for paraffin embedment as previously described ENRfu (Wheeler et al., 1998). Briefly, embryos were washed in phosphate buffered saline (PBS) and dehydrated through graded ethanols and xylene infiltrated. Embedded embryos were cut into 10 μm-thick sections and mounted on Superfrost Plus glass microscope slides in a 42° C. waterbath. Slides containing tissue sections were baked overnight at 45° C. and re-hydrated in PBS prior to, prehybridization.

RNA Probe

First strand cDNA was synthesized from isolated total mouse heart RNA using oligo-dT primers. The cDNA was subjected to PCR using the sense and antisense primers 5′CGAACTGCTCAATGCTCTCCGC3′ and 5′CCCCGATGCCTCCGCTAACC3′, respectively. Specific RNA probes for βIG-H3 were developed using a 1259 bp cDNA strand corresponding to nucleotides 540-1798 (GenBank accession number L19932). A BLAST search ENRfu (Altschul et al., 1990) of our probe sequence was performed and homology to other gene sequences was not detected. The 1259-bp amplified product was subcloned into pGEM-T. The plasmid was made linear with NotI (antisense) and SacII (sense) to obtain DNA templates. ³⁵S-rUTP—labeled antisense and sense probes were transcribed from the cDNA using the T7 and SP6 RNA Polymerase Riboprobe Combination System.

In Situ Hybridization

In situ hybridization reactions were performed according to a previously described protocol ENRfu (Wheeler et al., 1998). Care was taken to keep all tissue sections and laboratory equipment free of active ribonuclease. DEPC-treated water was used in all prehybridization washes. Unless otherwise specified, all procedures were performed at ambient temperature. Sections of paraffin-embedded embryos were mounted on slides, de-paraffinized in xylene and rehydrated through graded ethanols. The sections were immersed in 4% paraformaldehyde, pH 7.4, for 5 minutes and then washed in fresh PBS. Sections were treated for 8 minutes with 20 μg/ml Proteinase K and immersed again for 5 minutes in 4% paraformaldehyde solution. Positive charges on the tissue sections were blocked with acetic anhydride in 0.1 M triethanolamine and sections immersed in 2×SSPE and dehydrated in graded alcohol as a preparation for probe hybridization.

Probes were diluted to 525,000 dpm/slide in Prehybridization Solution A (2×SSPE, 50% formamide, 5 mM EDTA, 20 mM DTT and 20 mM Tris, pH 7.4) supplemented with 1×Denhardt's reagent, containing dextran sulfate to yield 10% (w/v) and 500 μg/ml yeast tRNA. A 50 ml volume of this solution was spread over each mounted embryo and protected with glass coverslips. Probes were hybridized to the tissue sections at 50° C. in a moist chamber for 16 hours followed by rinsing with 2×SSC and a 10-minute wash with Prehybridization Solution A at 60° C. To reduce background signal, the sections were treated with 10 μg/ml RNase A at 37° C. for 30 minutes. RNase A digestion was followed by a rinse with 2×SSC (30 minutes), 0. 1×SSC (15 minutes, 60° C.) and 0.1×SSC (30 minutes). Sections were dehydrated in graded ethanols containing 0.3 M ammonium acetate, dried overnight, and dipped in NTB-2 photographic emulsion. All sections were exposed for 3 weeks at 4° C. In some experiments, serial sections of tissue were counterstained with methyl green. Sections were analyzed by darkfield and phase-contrast microscopy with a Nikon Eclipse 600.

Cell Culture

Cell types used in this investigation were cultured in their respective growth media supplemented with 50 μg/ml penicillin and 50 μg/ml streptomycin sulfate. All cells were maintained at 37° C. in 95% ambient air and 5% CO₂ and were tested for mycoplasma by an ELISA-based methodology (Russell, 1975) and found to be negative.

Corneal Epithelial Cells

An SV-40 transfected corneal epithelial cell line was utilized in attachment assays. Previous characterization of this cell line showed similar properties to normal corneal epithelial cells, including desmosome formation, keratin expression, and stratification (Araki-Sasaki, 1995). Corneal epithelial cells were cultured in SHEM medium composed of a 1:1 mixture of DMEM and Hams F-12 media. SHEM medium was supplemented with insulin (5 μg/ml), choleratoxin (1 μg/ml), human EGF (10 ng/ml), gentamycin (40 μg/ml), and 10% FBS. Stromal Fibroblasts, Dermal Fibroblasts, and Myofibroblasts

Rabbit corneal fibroblast strains and primary human dermal fibroblasts were cultured as described previously in DMEM supplemented with 10% FBS ENRfu (Barry-Lane et al., 1997; LeBaron et al., 1995). TRK-36 comeal fibroblasts appear similar to normal corneal fibroblasts, maintaining a stellate, keratocyte morphology when grown in the absence of serum. TRK-43 corneal fibroblasts show evidence of a wound response phenotype and features that are characteristic of myofibroblasts, expressing α-smooth actin under serum-free conditions.

Murine Myoblasts and Osteoblasts

C2C12 murine myoblasts (ATCC number CRL-1772) were cultured as monolayers as previously described ENRfu (Yaffe and Saxel, 1977). Cells were maintained in DMEM supplemented with 10% heat-treated FBS. Primary myoblasts were isolated from the quadricep muscle obtained from mouse embryos (CD-1 strain, day 17.5). The tissue was dissected from both hindlimbs, trypsinized for ten minutes at 37° C., and seeded onto a substratum comprised of 50 μg/ml type I collagen flooded with F10 growth medium. Chick embryo extract (30 μl/ml), insulin (10 ng/ml), and bFGF (20 ng/ml) were added daily to the F10 growth media plus 10% horse serum to maintain myoblast population and to prevent differentiation. Cells below passage seven were used in solid phase attachment assays. Murine osteoblast 2T3 cells were a gift from Dr. Stephen E. Harris, University of Texas Health Science Center, San Antonio ENRfu (Ghosh-Choudhury et al., 1996). The 2T3 cells were maintained in α-MEM supplemented with 10% FBS.

Recombinant βIG-H3

Serum-free medium (IS CHO-V-GS) conditioned by Chinese hamster ovary (CHO) cells expressing βIG-H3 ENRfu (LeBaron et al., 1995; Skonier et al., 1994) was centrifuged to remove debris. The supernatant was exchanged for water and concentrated over a flow cell YM membrane (cutoff 30,000), lyophilized and stored at −20° C. βIG-H3 was purified by rehydration of the lyophilizedretentate in 50 mMNaCl, 50 mM Tris, pH 8.0, and application sequentially over heparin-agarose and hydroxyapatite. Elution buffers were: Buffer A; 50 mM Tris, 10 mM NaCl, pH 5.5; Buffer B, Buffer A containing 1 M NaCl; Buffer C, 10 mM NaPO₄ buffer, pH 6.8; Buffer D, 0.4 M NaPO₄ buffer, pH 6.8. Coomassie Blue-stained protein acrylamide gels and immunoblot analyses confirmed homogeneity of the βIG-H3 preparation. Bicinchoninic acid assay ENRfu (Smith et al., 1985) was utilized to determine the concentration of purified βIG-H3 for experiments.

Solid-Phase Cell Attachment Assay

Substratum comprised of 10 μg/ml βIG-H3 was prepared by coating multi-well plates as described ENRfu (LeBaron et al., 1995). Wells coated with 10% BSA and 10 μg/ml type I collagen served as control substrata. Sub-confluent cell monolayers were pre-incubated with 10 μg/ml cycloheximide in serum-free media for one hour and detached by the addition of 0.1 M EDTA in PBS. Cells were washed, re-suspended in their appropriate serum-free medium containing cycloheximide and seeded at a density of 4×10⁴/well. After a one-hour incubation, non-attached cells were rinsed from wells. The number of attached cells were quantified by addition of WST-1 and absorbance at 450 nm recorded. To determine statistical significance, three experiments (duplicate wells in each individual experiment) ±SD were evaluated and significance calculated by paired t-tests. Differences were considered significant when p≦0.05.

RESULTS

Structure and Organization of βig-H3

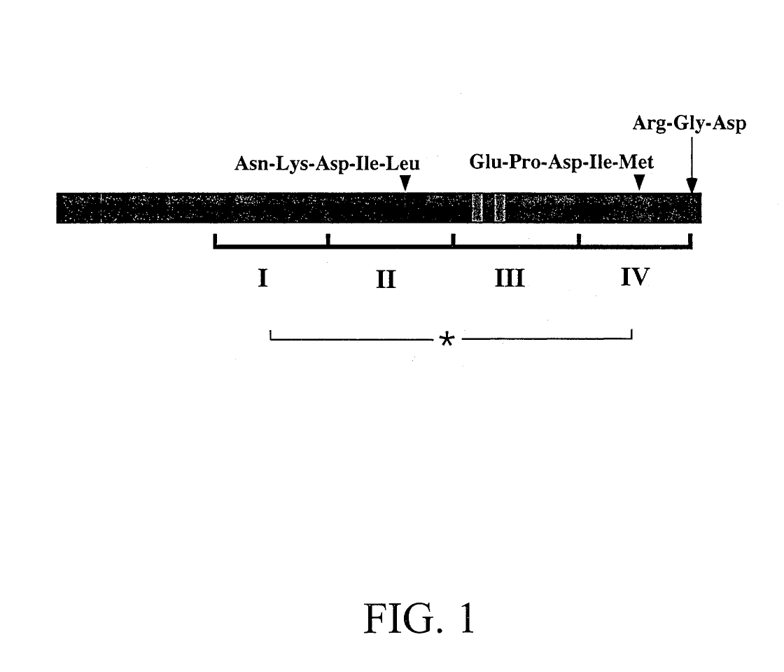

Shown in FIG. 1 is an analysis of the cDNA deduced amino acid sequence of βIG-H3. Schematically identified are four internal repeats sharing homology with fasciclin I ENRfu (Skonier et al., 1992; Zinn et al., 1988). Our analysis of the amino acid sequence revealed that βIG-H3 contains two putative heparin-binding sequences within the third fasciclin-like repeat. These potential heparin-binding motifs are potentially functional because human recombinant βIG-H3 binds to heparin-agarose in vitro (data not shown). Additionally, an Arg-Gly-Asp tripeptide sequence is within the fourth fasciclin-like repeat ENRfu (Skonier et al., 1992) and the sequences Asn-Lys-Asp-Ile-Leu and Glu-Pro-Asp-Ile-Met, reported to mediate cell adhesion ENRfu (Kim et al., 2000b) are within the second and fourth fasciclin-like repeats, respectively. An antisense probe corresponding to the region indicated (FIG. 1) was utilized to screen for specific spatiotemporal patterns of βIG-H3 transcripts in mouse embryologic tissue (E12.5-E18.5 p.c.).

Chondrogenic Tissues

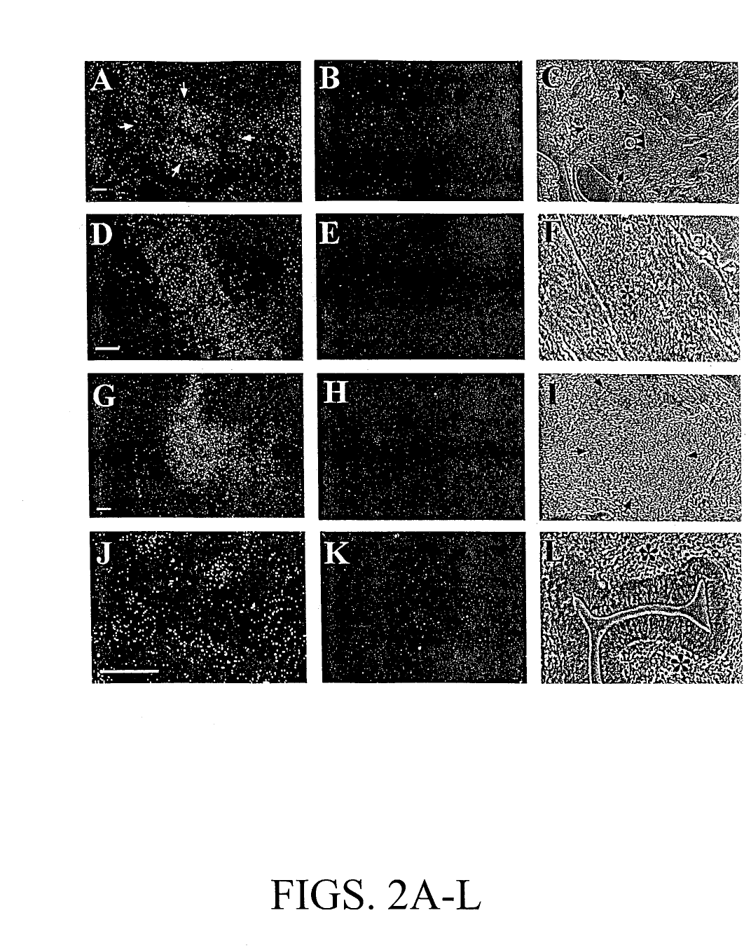

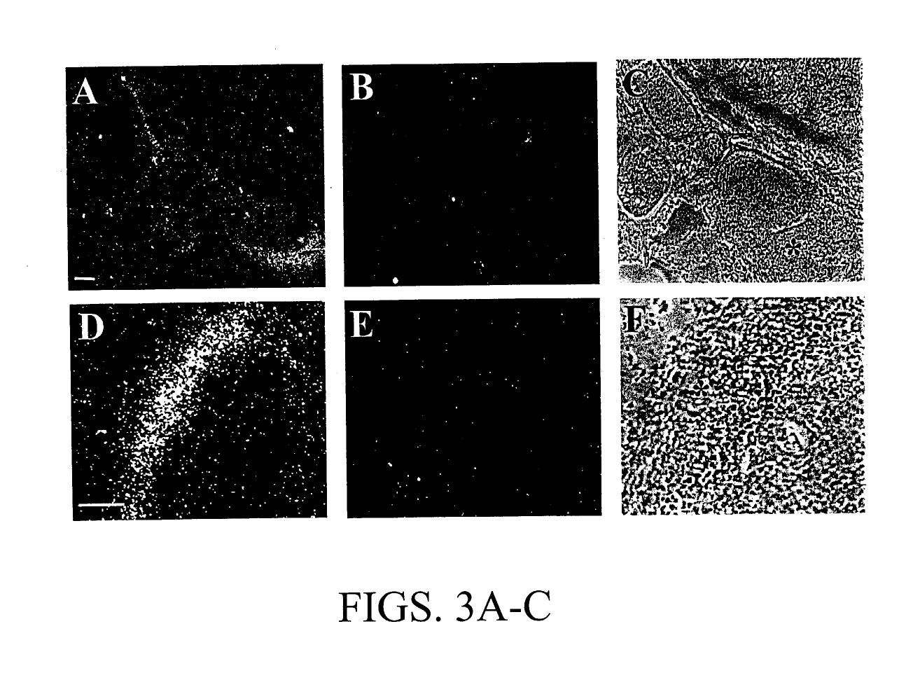

Prominent expression of βIG-H3 transcripts were detected in chondrogenic tissue destined for cartilage or bone formation. As early as E12.5, pre-chondrocytic mesenchymal cells expressed βIG-H3 transcripts abundantly. This expression occurred during the cell recruitment stage for the formation of the cartilaginous model. βIG-H3 expression was observed in all developing areas of axial, craniofacial, and appendicular primordial cartilage, including areas of vertebral cartilage (FIG. 2A). Cells within the cartilage primordia of E13.5 ribs expressed βIG-H3 transcripts (FIG. 2D), as did cells of the upper limb cartilage (FIG. 2G). Expression of βIG-H3 was also observed in cranial cartilage development, as shown in the nasal cartilage primordia (FIG. 2J). During E12.5, expression of βIG-H3 was also clearly evident in cranial and vertebral growth plates (FIGS. 3A and 3D), which are areas of cell proliferation (Farnum et al, 1990).

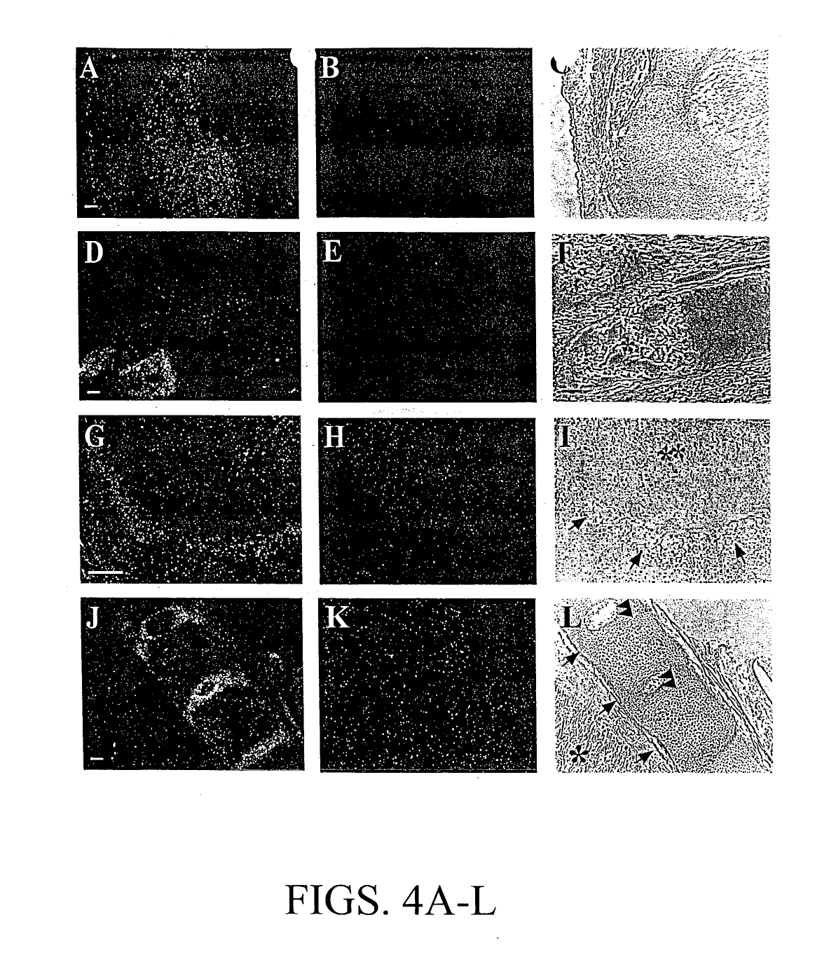

Expression of βIG-H3 transcripts continued until E15.5 when cartilage had taken shape into the future appendicular and axial skeleton. Proliferating, interior chondrocytes in the E16.5 scapula expressed βIG-H3 transcripts (FIG. 4A). At days E16.5-E17.5, βIG-H3 transcripts were observed within specific areas of tissue destined for ossified bone. βIG-H3 message localized to regions of endochondral ossification initiation, shown here in the tibia of an E17.5 embryo (FIG. 4D), as well as intramembranous ossification, such as the formation of bone surrounding the temporal lobe of the embryonic brain (FIG. 4G). In the cartilage models, βIG-H3 transcripts were abundant in regions of proliferating chondrocytes but not in regions occupied by hypertrophic chondroctyes (FIG. 4J).

Joint Formation

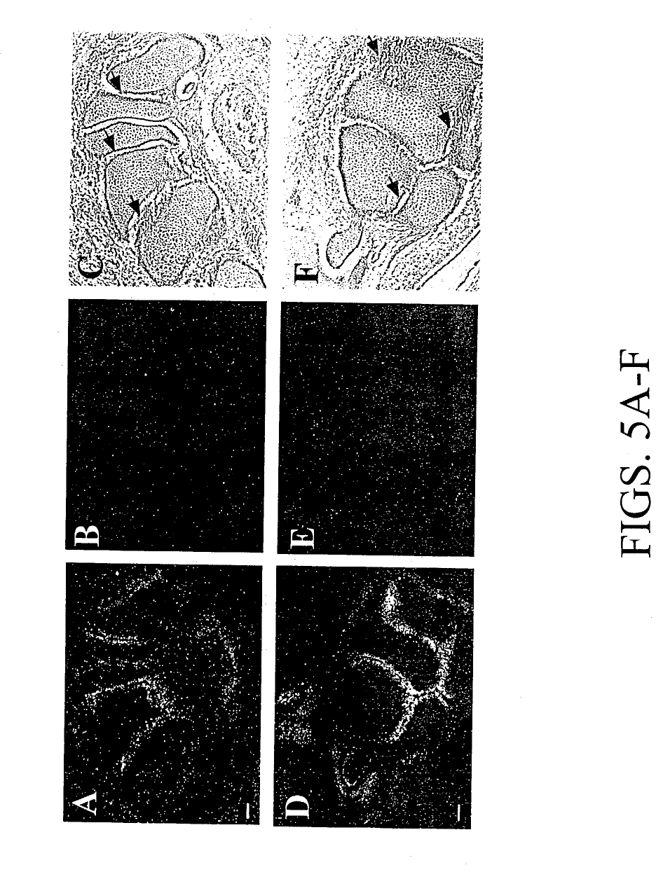

Transcripts were identified at developing joint structures beginning at E16.5 and becoming more evident during E17.5-E18.5. Transcripts were observed at stages that were concurrent with articulating ends of bones capped with hyaline cartilage. These included cartilage primordia in the mouse footpad at E17.5 (FIG. 5A) and between the developing bones in the hindlimbs at E18.5 (FIG. 5D). Expression was moderate at E15.5 but steadily increased in intensity in subsequent older embryonic stages.

Fibrous Capsules

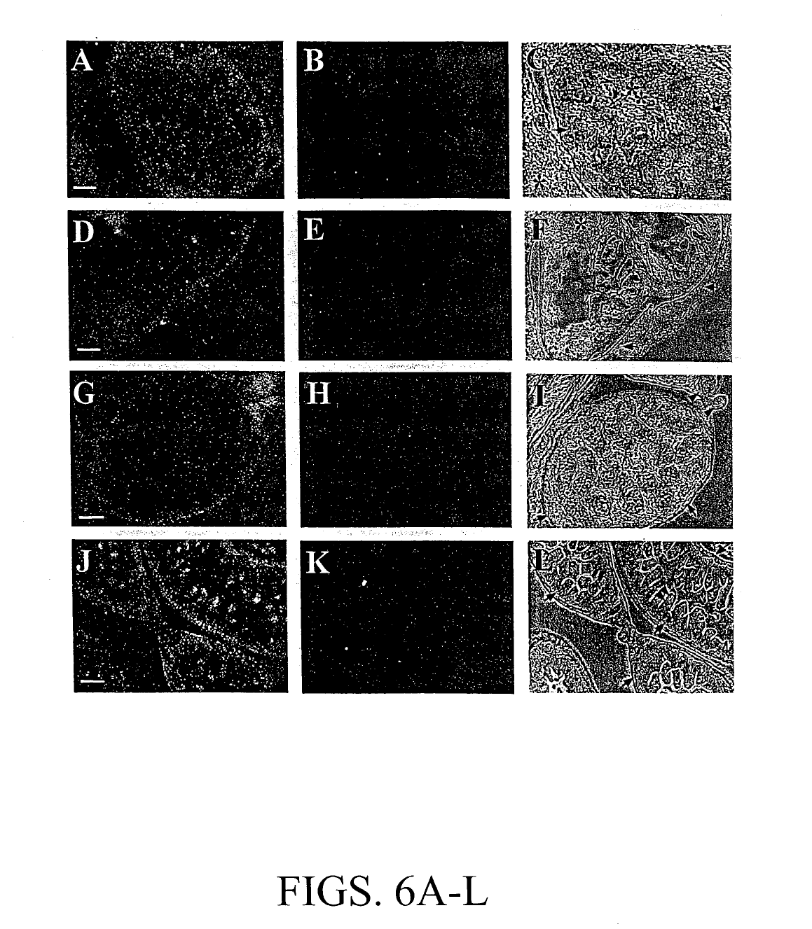

Abundant expression of βIG-H3 message was observed in several connective tissue capsules, including the fibrous capsule of the kidney at E12.5, which continued to express βIG-H3 transcripts until E18.5 (FIG. 6A). Transcripts were also localized throughout the medulla mesenchyme of the kidney during E12.5-E18.5, specifically in the connective tissue regions surrounding areas of the glomeruli formation. Another fibrous connective tissue, the pleural pericardium membrane surrounding the developing heart, moderately expressed βIG-H3 transcripts from E12.5-E15.5 (FIG. 6D).

The testes' tunica albuginea, another dense connective tissue capsule, also expressed βIG-H3 transcripts from E12.5-E17.5 (FIG. 6G). Relative to the tunica albuginea, an intense expression of βIG-H3 transcripts was evident in the rete testis and the mediastinum of the testes, a local thickening of the tunica albuginea where seminiferous tubules converge before leaving the testes.

Within the serosa (the connective tissue capsule of the gut wall), a striking intensity of the βIG-H3 probe was evident (FIG. 6J). Additional areas in the digestive tract displayed prominent βIG-H3 transcript levels as well, including the muscularis externa layers consisting of smooth muscle intertwined with autonomic nerve fibers that contribute to peristaltic motions. The lamina propia, which supports the interior epithelial layer of the intestinal tract and brings blood and lymphatic capillaries close to the epithelium, also expressed βIG-H3 transcripts. The expression in the intestines was abundant beginning at E12.5 but declined to moderate levels by E15.5 and remained moderate until E18.5 (data not shown).

Therefore, the cell-cell interactions occurring in the muscularis externa (smooth muscle with nerve fibers), lamina propia (epithelium and capillaries) and the pleural pericardium (pericardium with heart) were accompanied by abundant expression of βIG-H3. Overall, results reveal that βIG-H3 expression occurs in regions of the tissues that undergo active morphogenesis or tubular extension, typically involving a remodeling of the associated ECM. A possible role for βIG-H3 may be one in mediating cell-cell or cell-substratum contacts that are generated during the development of these tissues.

Epithelial-Mesenchymal Interactions

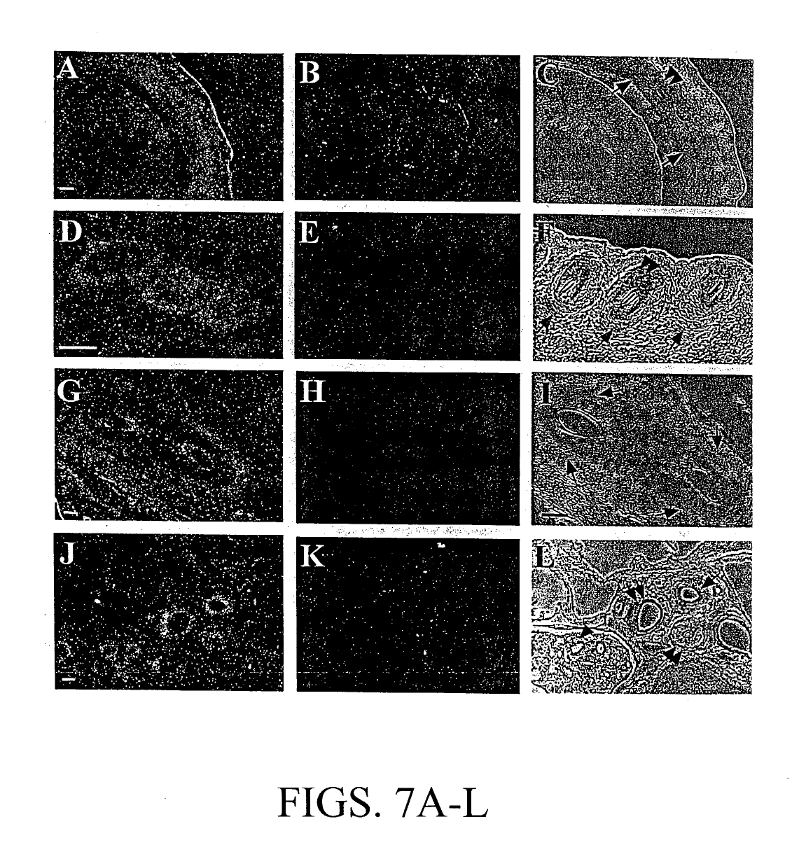

βIG-H3 transcripts were observed in or around numerous other tissues, including tissues involved in epithelial-mesenchymal induction mechanisms. Transcripts were observed in the surrounding mesenchyme of the cochlea (E13.3-E18.5), the corneal epithelium (E15.5-E18.5), corneal stroma (E15.5-E18.5), vibrissae (E14.5-E18.5), and the cartilaginous bronchi of the developing lungs (E12.5-E18.5). Expression in the corneabegan around E15.5 and continued until E18.5, becoming more abundant as development progressed. Transcripts were localized to both the corneal stroma and in the epithelial layer (FIG. 7A). Near vibrissae, βIG-H3 expression was mainly present in the condensed mesenchyme of the dermis surrounding the epithelial layer of the hair follicle (FIG. 7D). Moderate transcript expression localized to the overlying epidermis.

The mesenchyme surrounding the cochlear duct contained βIG-H3 transcript levels that peaked at E13.5 and then declined through E18.5 (FIG. 7G). βIG-H3 expression was also evident in lung, including mesenchyme surrounding the epithelial layer of the developing bronchi (FIG. 7J). βIG-H3 transcripts in the lung were expressed in early stages moderately, becoming increasingly more intense by E18.5. Displaying moderate βIG-H3 expression were muscularis externae surrounding the esophagus, mesenchymal tissue surrounding the aorta and the hyaline cartilage rings surrounding the developing trachea (FIG. 7J).

During development, interactions between mesenchyme and epithelium are involved in tissue morphogenesis and induction of cell differentiation into specified phenotypes. The expression of βIG-H3 was coincident with these interactions and was expressed at the highest levels when tissue remodeling was the most active. Again, a role for cell-cell or cell-substratum interactions is indicated.

Connective Matter Surrounding Neural Tissue

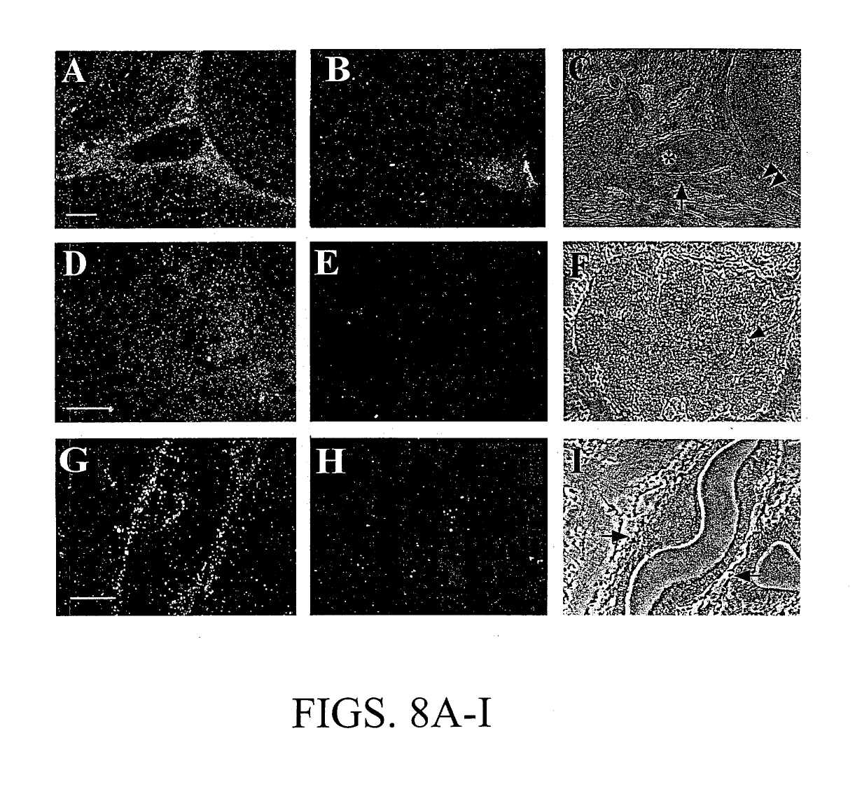

As observed with most connective tissue within the mouse embryo, strong βIG-H3 expression was also evident in connective tissue surrounding specific neural tissue. Expression was localized to the layers of the dura mater surrounding the optic nerve stalk from E14.5-E18.5 (FIG. 8A). The sclera of the eyeball expressed βIG-H3 within the same time frame (FIG. 8A). The sclera, composed of fibroblasts and dense fibrous collagen tissue intermingled with fine elastic fibers, fuses with the dural and arachnoid sheaths of the optic nerve at these stages. Interestingly, significant levels of βIG-H3 transcripts were observed throughout the trigeminal ganglia at E14.5, with perhaps an increased signal intensity within the caudal half of the ganglia (FIG. 8D). An additional area of βIG-H3 expression observed at E14.5 was the surrounding epithelial wall of Rathke's pouch, within an area of intense cell proliferation (FIG. 8G).

Muscle Tissue

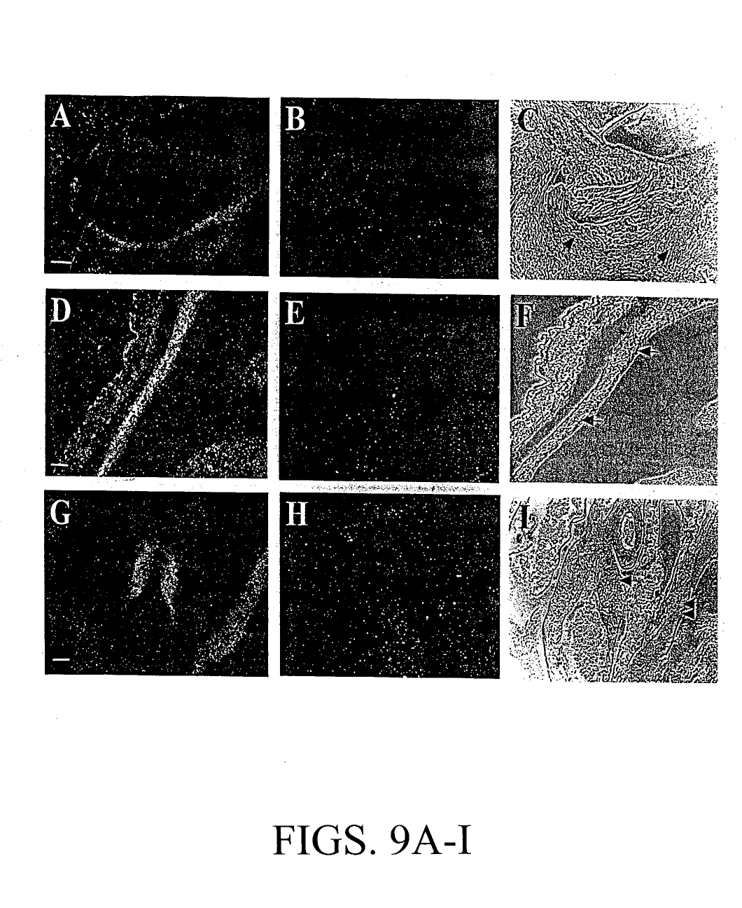

βIG-H3 transcripts were evident in the epimysium surrounding muscle fiber bundles in later stages (FIG. 9A). In more specialized muscle tissue such as the diaphragm muscle, βIG-H3 transcripts were abundantly expressed along the entire surface from E15.5-E17.5 (FIG. 9D). Cardiac valves in the embryonic heart expressed βIG-H3 transcripts from E12.5, increasing to a maximum intensity at E14.5 (FIG. 9G). Transcript levels in the valves then began a slight decline from E15.5 until E18.5. Transcripts were also evident in all sections where muscle contacted developing bone, an expansion of this finding demonstrated that bIG-H3 protein is expressed markedly at developing and adult myotendinous junctions (manuscript in preparation).

βIG-H3 In Vitro Mediates Cell Adhesion

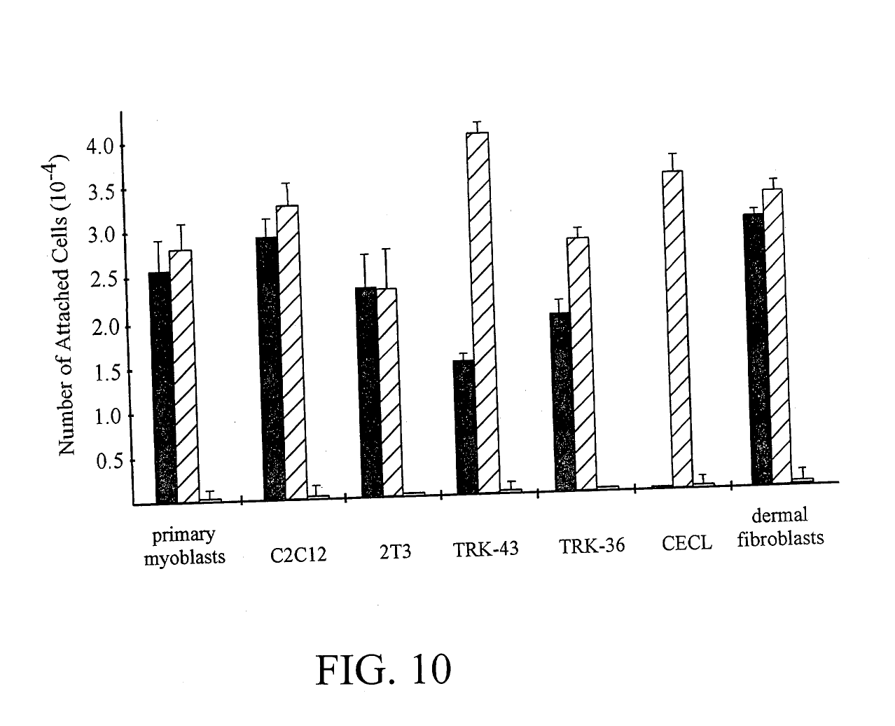

The distribution of βIG-H3 mRNA throughout development was concordant with a possible role of βIG-H3 in cell adhesion. The tissue distribution suggests that cells interacting with βIG-H3 may include non-differentiated mesenchymal cells and also differentiated cell types including chondrocytes, osteoblasts, fibroblasts, skeletal muscle cells, and epithelial cells. Therefore, whether a substratum that is comprised of purified recombinant βIG-H3 would support the attachment of various cell types was investigated . Here, solid phase binding assays were performed with cell types that expressed βIG-H3. As a control cell type, primary human dermal fibroblasts were included because they have been shown to bind βIG-H3 ENRfu (LeBaron et al., 1995). To prevent endogenous protein translation from affecting the outcome of our experiments, cells were pretreated with the protein synthesis inhibitor cycloheximide. The results demonstrate that a substratum comprised of purified recombinant βIG-H3 supported adhesion of osteoblasts, stromal fibroblasts and skeletal muscle cells. The notable exception was corneal epithelial cells, albeit the epithelial cells readily attached to type I collagen (FIG. 10). Few, if any cells attached to BSA.

Discussion

This study reveals evidence of two new findings. One finding is that βIG-H3 expression appears to be spatially restricted during development, localized principally within and near tissues enriched in collagens and elastin. A second finding is that βIG-H3 serves in vitro as an adhesion substratum for some, but not all cells types that correlate with spatial expression of βIG-H3. A developmental model was chosen because tissue modeling involves extensive changes in ECM biosynthesis that can be correlated with developmental events and thus facilitate discovery of function. This investigation tested for βIG-H3 transcripts during the developmental stages of E12.5-E18.5. These stages ofdevelopment were selected because RNA blot analysis indicated that βIG-H3 transcripts were not expressed until after E12 of murine development ENRfu (Skonier et al., 1992) and βIG-H3 was observed in ocular tissue at E18.5 ENRfu (Schorderet et al., 2000). This study identifies additional tissues that express βIG-H3 transcripts and documents a biological activity of βIG-H3, predicted from information obtained by protein sequence analyses and the spatiotemporal distribution of βIG-H3 transcripts.

Results suggest that βIG-H3 plays an important role in various embryonic tissues, particularly those of mesodermic and neural crest origin, at specific times when critical morphogenic events occur. A general conclusion can be drawn that βIG-H3 appeared closely-associated with mesenchyme per se or with tissues derived from mesenchyme, such as cartilage and bone. Embryonic mesenchymal cells (EMCs) are fundamentally important in the overall classification of supportive tissue. Not only can EMCs self-replicate, they are also capable of differentiating into fibroblasts, chondroblasts, myoblasts, and osteoblasts (Bianco, 1998). Our results suggest that βIG-H3 transcripts appeared in areas occupied by EMCs during the development of the mouse embryo.

Generally, βIG-H3 transcripts were detected during several steps leading toward formation of mature bone, including primary morphogenesis of the cartilage model in earlier stages and subsequent chondrogenesis and ossification. The formation of bone consists of a series of cellular activities, including chemotactic, proliferative, and differentiative stages. On the tissue level, this consists of a sequence of modeling and remodeling events, until the final mature structure is formed (Ballabriga, 2000).

Transcripts of βIG-H3 were observed during the formation of axial and appendicular bones, most notably at stages of cell recruitment, increased matrix secretion, and differentiation. Axial and appendicular bones of the skeleton are derived from the mesoderm. Their development is indirect in that they undergo endochondral ossification (DeLise et al., 2000; Ballabriga et al., 2000), a process where a cartilage model of chondrocytes exists prior to differentiation and osteogenesis. Marked expression of βIG-H3 was observed during the assembly and recruitment of mesodermic cells when forming the cartilage primordial model. During the proliferative phase of chondrocytes prior to bone formation, chondrocytes secrete large amounts of ECM, contributing to the growth and expansion of the cartilage model (Moss et al., 1983). Transcripts of βIG-H3 were expressed by proliferating chondrocytes, however, expression was reduced significantly in subsequent hypertrophic stages. During the late stages of bone development (E17.5-E18.5), βIG-H3 transcripts were again detected. Additionally, in later stages of joint formation, such as articulations between bones, a relative abundance of βIG-H3 transcripts was evident.

Previous data showing that in vitro βIG-H3 supports attachment of chondrocytes ENRfu (Ohno et al., 1999) and binds collagen ENRfu (Hashimoto et al., 1997; Rawe et al., 1997) is consistent with the expression of βIG-H3 transcripts in the regions detected in the present study. Additionally, expression of βIG-H3 MRNA is potentially regulated by factors influencing cell differentiation. Treatment of human bone marrow stromal cells in vitro with dexamethasone promotes osteogenic differentiation. Relative to several different connective tissue cell types, dexamethasone treated bone marrow stromal cells exhibited a marked decrease in βIG-H3 mRNA and a suggestion was put forth that βIG-H3 is a negative regulator these cells ENRfu (Dieudonne et al., 1999).

The expression of βIG-H3 transcripts in cartilage and bone formation was not limited to the axial and appendicular skeleton, however, as the synthesis of transcripts was also conspicuous in mesenchyme of neural crest origin. These included such tissues as craniofacial cartilage mesenchyme and bone, nasal sinuses and nasal cartilage, and the connective tissue forming the dura mater. Unlike the endochondral ossification process observed in limbs, the cranial, facial flat bones and mandibles evolve directly in areas of vascularized neural crest mesenchyme in a process called intramembranous ossification (Ballabriga et al., 2000). It is speculated that TGF-β1, also expressed in neural crest mesenchyme, increases the levels of ECM molecules that may contribute to the migration of neural crest cells and their subsequent differentiation in becoming craniofacial mesenchyme ENRfu (Heine et al., 1987). In a number of different cell types, βIG-H3 expression is induced by TGF-β1 ENRfu (Dieudonne et al., 1999; Gilbert et al., 1998; Hashimoto et al., 1997; Kim et al., 2000a; LeBaron et al., 1995; Skonier et al., 1994; Skonier et al., 1992) and may therefore be involved in mediating some of the varying effects that TGF-β1 evokes. Spatiotemporal similarities between βIG-H3 and TGF-β1 expression were striking in the mouse embryo, and an accumulation of βIG-H3 expression was generally observed in or near tissues whose cells express TGF-β1 in vivo. Indeed, the expression patterns of βIG-H3 appeared to essentially mirror previously documented TGF-β1 expression within the mouse embryo ENRfu (Heine et al., 1987; Thompson et al., 1989).

The paucity of βIG-H3 expression within the nervous system is consistent with previous analysis of brain tissue ENRfu (Skonier et al., 1992) and paralleling expression patterns of TGF-β1 ENRfu (Heine et al., 1987). However, βIG-H3 mRNA was detected in the dural layers surrounding the brain, the spinal cord, and the optic nerve; structures that correspond to areas of limited TGF-β1 expression ENRfu (Heine et al., 1987). βIG-H3 transcripts were localized to specific tissues typically identified with sensory nerve innervation and sensation. The trigeminal nerve contributes to sensation received from the skin of the face and forehead, the cornea, the mucosa of the oral and nasal cavities, and from the dura mater (Barr et al., 1988). βIG-H3 transcripts in the dura mater was observed, regions of developing vibrissae, the cornea, and the tongue. All of these tissues are infiltrated with nerves emanating from one of the three branches of Cranial Nerve V (trigeminal nerve). Interestingly, βIG-H3 transcripts were found within the trigeminal ganglia itself. Expression was observed in the caudal half of the ganglia, an area where the ophthalmic (V₁), maxillary (V₂), and mandibular (V₃) nerve divisions exit. This suggests that βIG-H3 may have a role in specific neuronal cell and processes movement, however whether βIG-H3 is involved with neural cell migration remains an open question presently.

Although most βIG-H3 message was mainly observed in tissue of mesenchymal origin, in some instances, βIG-H3 transcripts localized to epithelium. Epithelia expression appeared to be restricted to times and areas where critical interactions between mesenchyme and epithelial tissue occur, including during the development of vibrissac, bronchi of the lung, and the cornea. In tissue containing vibrissae, βIG-H3 expression was mainly present in the condensed mesenchyme of the dermis surrounding the epithelial layer of the hair follicle. βIG-H3 expression in lung was similar in that expression was also in mesenchyme surrounding the epithelial layer of the developing bronchi.

Expression of βIG-H3 was evident in areas of angiogenesis and the formation of vasculature. Connective tissue walls of several large vessels, including the vena cava and aorta, expressed βIG-H3. Areas rich in capillary infiltration, such as endochondral and intramembranous ossification, also displayed βIG-H3 transcripts. Furthermore, βIG-H3 transcripts appeared during the formation of primordial heart valves, which are derived from the mesenchyme of endocardial cushion tissue. The endocardial cushions form in the early heart as swellings rich in types I and III collagen, fibronectin, and proteoglycans ENRfu (Fitzharris and Markwald, 1982). This particular connective tissue is involved in the modeling ofthe heart, leading to the partitioning ofthe atrioventricular canal and the formation of the connective tissue framework of the cardiac valves ENRfu (Icardo and Manasek, 1984).

Inmunohistochemical analyses of βIG-H3 expressed by various cells demonstrated that anti-βIG-H3 antibody was detected on the cell surface and within the ECM (not shown). The immunolocalizations, considered together with the presence of putative cell-binding sequences in βIG-H3, suggested a biological role involved with mediating attachment of various cell types. Thus, an in vitro solid phase cell attachment model system was utilized to examine cell attachment onto recombinant βIG-H3 of a subset of cell types that expressed βIG-H3 mRNA during mouse development. The adhesion of these cells was examined under conditions limiting biosynthesis of endogenous proteins by including cycloheximide in the experiments. Results of the cell adhesion assays demonstrated that in vitro, a substratum comprised of βIG-H3 supported the adhesion of several cell types derived from mesenchyme, suggesting that during developmental processes, βIG-H3 may mediate cell adhesion. This finding is consistent with the possibility that βIG-H3 plays a role in tissue organization and modeling, perhaps bridging interactions between cells, collagens and proteoglycans.

Generally, the principal expression patterns of βIG-H3 transcripts during murine development suggest βIG-H3 protein associates with several mesenchymal and epithelial cell types. Additionally, the detection of βIG-H3 transcripts within collagen-rich tissues is consistent with the findings that in vitro, βIG-H3 may associate with collagen types I, II, IV and VI ENRfu (Hashimoto et al., 1997; Rawe et al., 1997). βIG-H3 may link collagens and various other components of the ECM and cells, including fibrillin-containing elastic fibers ENRfu (Gibson et al., 1996), fibroblasts IN ENRfu (LeBaron et al., 1995), bladder cells ENRfu (Billings et al., 2000), osteogenic cells ENRfu (Dieudonne et al., 1999; Ohno et al., 1999), and epithelial cells ENRfu (Kim et al., 2000b), perhaps playing a cell adhesion and structural role. Although the normal, physiological function of βIG-H3 is not yet well understood, mounting evidence suggests that βIG-H3 contributes toward development of several different pathologies. The βIG-H3 gene has been localized to human chromosome 5q31, where a deletion of this locus is the most common lesion found in myelodysplastic syndrome subtypes and many human leukemias. Consequently, 5q31 has been suggested to contain a tumor suppressor gene ENRfu (Pedersen and Jensen, 1991) of which βIG-H3 is a candidate ENRfu (Skonier et al., 1994; Skonier et al., 1992). The 5q31 locus is also proposed to contain genes in which a lesion may contribute to Limb Girdle Muscular Dystrophy Type 1A ENRfu (Horrigan et al., 1999). Interestingly, anti-βIG-H3 antibody exhibited a marked staining at myotendinous junctions in embryonic and adult tissues. Thus the βIG-H3 gene and its protein product are spatially poised to possibly contribute to the development of muscular disorders. Along these lines, mutations in the βIG-H3 gene that results in single amino acid changes in βIG-H3 protein appear to be causative in the development of corneal dystrophies ENRfu (Korvatska et al., 1999; Munier et al., 1997). Aberrant expression of βIG-H3 may contribute toward development of vascular lesions ENRfu (O'Brien et al., 1996), and fibrous tissue accumulation in the juxtaglomerular apparatus in experimentally induced diabetic rats ENRfu (Gilbert et al., 1998). βIG-H3 serves as an adhesion substratum for various types of cells derived from mesenchyme (this study, but also see ENRfu (Hashimoto et al., 1997; LeBaron et al., 1995) and associates with collagens ENRfu (Hashimoto et al., 1997; Rawe et al., 1997). These findings, together with the possibility that mutant βIG-H3 plays a role in comel and muscular dystrophies, further supports the probability that βIG-H3 plays important structural and cell adhesion roles in tissue development and maintenance and calls for additional structural, biochemical and functional studies to elucidate the physiology of βIG-H3.

### Localization of βIG-H3 During Sketal Muscle Development

A secretary protein named Transforming Growth Factor BetaInduced Gene-Human Clone 3 (βIG-H3) contains sequences that are found in some extracellular matrix (ECM) adhesion molecules. We previously reported that βIG-H3 is expressed in mesenchymal-derived tissue and that in vitro, a substratum composed of human recombinant βIG-H3 promoted cell attachment. In this study, we have extended these initial observations and report that βIG-H3 is expressed at the myotendinous junction (MTJ) during E16.5-E18.5 of murine development. In situ hybridization experiments document prominent expression of βIG-H3 transcripts within regions where developing skeletal muscle fibers contact primordial cartilage and bone. Anti-βIG-H3 antibody localized distinctively at MTJs, predominately at the termini of myofibers. In vitro, βIG-H3 functioned as a cogent cell attachment substratum for skeletal muscle cells and exhibited an affinity for heparin. Cell adhesion was significantly reduced by function-antagonizing anti-integrin α7 antibody. The biological features of βIG-H3, including localization at termini of skeletal muscle fibers, association with cell adhesion receptors in vitro and an affinity for glycosaminoglycan, together with previous reports demonstrating a propensity to bind collagens, suggest that βIG-H3 may play an organizational and structural role in developing MTJs, linking skeletal muscle to components of the ECM. The human βIG-H3 gene is localized to 5q31, a region believed to contain mutations in patients diagnosed with Limb Girdle Muscular Dystrophy Type 1A (LGMD 1A). Thus, together with a localization of βIG-H3 at MTJs and its cell and ECM binding activities in vitro, aberrant βIG-H3 may play a role in development of LGMD 1A.

Introduction

The development of functional, healthy tissue depends on structures formed by cells and components of their ECM. Such arrangements are crucial in the early embryo, where various organizations of ECM and cells form distinctive structures. Muscle to bone union is organized and sustained by MTJs ENRfu (Benjamin and Ralphs, 2000), specialized structures comprised of various molecules that function together to bridge interactions between tendon and skeletal muscle. MTJs exhibit considerable tensile strength and serve to transmit forces generated by contracting and relaxing muscle to the skeletal system ENRfu (Tidball, 1991). Although sequential events leading to MTJ assembly and organization are in part understood ENRfu (Birk and Mayne, 1997; Tidball, 1994; Tidball and Lin, 1989), the precise molecular composition and arrangement of ECM within MTJs are not clearly delineated.

he biological functions of a secretory protein named βIG-H3 which was cloned and sequenced from a cDNA library constructed from mRNA isolated from human lung adenocarcinoma cells growth-arrested by treatment with TGF-β1 ENRfu (Skonier et al., 1992) were investigated.

The secretory protein sequence of human βIG-H3 is comprised of 683 amino acids with internal repeats similar to those found in fasciclin I ENRfu (Zinn et al., 1988), periostin ENRfu (Horiuchi et al., 1999), Algal-CAM ENRfu (Huber and Sumper, 1994), midline fasciclin ENRfu (Hu et al., 1998) and MPB70 ENRfu (Terasaka et al., 1989). An Arg-Gly-Asp tripeptide near the carboxy terminus implies that βIG-H3 possibly mediates cell adhesion through integrins ENRfu (Pierschbacher and Ruoslahti, 1984). Human dermal fibroblasts attached to a substratum comprised of recombinant βIG-H3 ENRfu (LeBaron et al., 1995), an activity that was subsequently observed for other cell types ENRfu (Ohno et al., 1999) ENRfu (Kim et al., 2000).

Biochemical evidence suggests that βIG-H3 may associate with microfibrillar proteins and collagens ENRfu (Gibson et al., 1996; Hashimoto et al., 1997; Hirano et al., 1996; Rawe et al., 1997). The prospect that in vivo, βIG-H3 binds cells and ECM molecules prompted us to consider the possibility that βIG-H3 plays a role in connective tissue biology. To address this view, we first tested for expression of βIG-H3 transcript and protein product in mouse embryos utilizing in situ hybridization and immunohistochemical methodologies. Our results document a striking expression of βIG-H3 at developing MTJs, where skeletal muscle fibers terminate at the tendon interface. Ultrastructural analysis of developing MTJs revealed βIG-H3 antibody localizes to the cell surface and associates with fibril-like structures within the extracellular space. The spatiotemporal expression of βIG-H3 in MTJs described in this study suggests a possible role in cell-substratum linkages during development. To investigate this suggestion, we tested recombinant βIG-H3 as a substratum for skeletal muscle cell attachment. Our results demonstrate that in vitro, skeletal muscle cells attach to a substratum comprised of βIG-H3; a process significantly reduced by function-perturbing anti-integrin antibody, leading to the conclusion that βIG-H3 may function as a component of structures linking skeletal muscle cells to tendon.

Materials and Methods

Materials

Oligo-dT primers used for first-strand cDNA synthesis were purchased from Boehringer-Mannheim (Indianopolis, Ind.). The transcription vector pGEM-T and T7/SP6 RNA Polymerase Riboprobe and probe reagents were from Promega Corporation (Madison, Wis.). Kodak Technical Panfilm (TP135-36) and NBT-2 photographic emulsion was obtained from Eastman Kodak (Rochester, N.Y.), Proteinase K was from Fisher Scientific (Pittsburgh, Pa.) and ³⁵S-rUTP from NEN Life Science Products Inc. (Boston, Mass.). Trypsin/EDTA was purchased from Mediatech, Inc. (Herndon, Va.). Immobilon P transfer membrane was obtained from Millipore (Bedford, Mass.). Glasgow's Minimum Essential Medium (GMEM) was purchased from ICN Biochemicals (Costa Mesa, Calif.). Dulbecco's Modified Eagle Medium (DMEM), Trizol LS, F10 Growth Medium, heat-inactivated horse serum, chick embryo extract and antibiotic reagents were purchased from Gibco BRL Life Technologies (Grand Island, N.Y.). Fetal bovine serum (FBS) was from Irvine Scientific (Santa Ana, Calif.). Human basic fibroblast growth factor (bFGF) was obtained from Peprotech (Rocky Hill, N.J.). Cell proliferation reagent 4-[3-(4-Iodophenyl)-2-(4-nitrophenyl)-2H-5-tetrazolio]-1,3-benzene disulfonate (WST-1) was from Roche Molecular Biochemicals (Indianopolis, Ind.). YM membrane was from Amicon, Inc. (Beverly, Mass.). Superfrost Plus pre-coated microscope slides and glass coverslips were obtained from VWR Scientific Products (Sugarland, Tex.). Anti-rabbit antibody conjugated to horseradish peroxidase (HRP) was from KPL Laboratories, (Gaithersburg, Md.). Accustain Trichrome Stain (Masson), methionine sulfoximine (MSX), bovine serum albumin (BSA), yeast tRNA, triethanolamine, insulin, cycloheximide, 3′,3′-Diaminobenzidine Tetrahydrochloride (DAB), heparin insolubilized on 4% beaded agarose, and all other reagent chemicals were from Sigma Chemical Company (St. Louis, Mo.). Function blocking antibodies to integrin subunits include anti-α7 antibody, clone CY8 ENRfu (Yao et al., 1997) and antibodies purchased from Pharmingen (San Diego, Calif.,; anti-β1 antibody, clone Ha2/5 ENRfu (Mendrick and Kelly, 1993), anti-α1 antibody, clone Ha31/8 ENRfu (Miyake et al., 1994), anti-α6 antibody, clone GoH3 ENRfu (Aumailley et al., 1990), anti-a5 antibody, clone 5H10-27 ENRfu (Schultz and Armant, 1995). Polyclonal antibodies used for protein blots and immunohistochemistry were generated against βIG-H3 bacterial fusion protein (amino acids 210-683) and previously characterized (Skonier, 1994; LeBaron, 1995).

Methods

Tissue Preparation

Mouse embryo stages E16.5-E18.5 were fixed in 10% formalin(E16.5-17.5, 24 hours, E18.5, 48 hours). Fixed embryos were prepared for paraffin embedding by graded ethanol dehydration followed by xylene infiltration. Sections (10 mm thick) of embedded embryos were floated in a 42° C. water bath and mounted onto Superfrost Plus glass microscope slides. Mounted tissue sections were baked overnight at 45° C. Prior to prehybridization, mounted sections were rehydrated and incubated in PBS for 10 minutes. Mice (strain CD-1) were obtained from Charles Rivers Laboratories (Wilmington, Mass.). All procedures were pre-approved by the Institutional Animal Care and Use Committee.

RNA Probe Generation

First strand cDNA was synthesized from total mouse heart RNA using oligo-dT primers. Reverse transcriptase-polymerase chain reaction was accomplished utilizing sense and antisense primer sequences comprised of 5′-CGAACTGCTCAATGCTCTCCGC-3′ and 5′CCCCGATGCCTCCGCTAACC-3′, respectively. Specific probes for βIG-H3 transcripts were developed using a 1259 bp cDNA strand corresponding to nucleotides 540-1798 of adult mouse heart βIG-H3 cDNA ENRfu (Skonier et al., 1994). The 1259-bp amplified product was subcloned into a pGEM-T transcription vector. The plasmid was linearized with NotI (antisense) and SacII (sense) to obtain cDNA templates. ³⁵S-rUTP—labeled antisense and sense probes were transcribed from the cDNA using the T7 and SP6 RNA Polymerase Riboprobe Combination System.

In Situ Hybridization

In situ hybridization reactions were performed as previously described ENRfu (Wheeler et al., 1998). Care was taken to keep all tissue sections and pertinent laboratory equipment RNase-free. DEPC-treated water was used in all prehybridization washes. Unless otherwise specified, all procedures were performed at ambient temperature. Thin sections of fixed embryos were mounted on slides, immersed in 4% paraformaldehyde for 5 minutes and then washed in PBS. Sections were treated for 8 minutes with 20 mg/ml Proteinase K and immersed again for 5 minutes in 4% paraformaldehyde solution. Tissue sections were blocked with acetic anhydride in 0.1M triethanolamine, and sections immersed in 2×SSPE and dehydrated in graded alcohol as a preparation for probe hybridization.

Probes were diluted to 525,000 dpm/slide in 20 mM Tris, pH 7.4 containing 2×SSPE, 50% formamide, 5 mM EDTA, 20 mM DTT and 500 μg/ml yeast tRNA. Denhardt's reagent containing 10% dextran sulfate (w/v) was designated Prehybridization Solution A. A 50 μl volume of Prehybridization Solution A was spread over each mounted embryo and secured with glass coverslips. Probes were hybridized to the tissue sections at 50° C. in a moist chamber for 16 hours. Nonspecific binding of probe was removed by rinsing tissue with Prehybridization Solution A at 60° C. followed by rinses with 2×SSC, 0.1×SSC (60° C.), and 0.1×SSC. To reduce background signal, the sections were treated with 10 mg/ml RNase A at 37° C. for 30 minutes. Sections were dehydrated to 100% ethanol containing 0.3M ammonium acetate, dried overnight, and dipped in NTB-2 photographic emulsion. Sections were exposed for 3 weeks at 4° C. and counterstained with methyl green. In some experiments, serial sections were stained with Masson's trichrome stain.

Immunohistochemical Analysis

Sections of mouse embryo stages E16.5-E18.5 were prepared as described above, rehydrated in PBS and treated with 0.1% trypsin at 37° C. for 15 minutes. Endogenous peroxidase activity was neutralized by incubation for 20 minutes in a 1:4 solution of 30% hydrogen peroxide and methanol. Following a PBS wash, the sections were incubated for one hour with 1% BSA in PBS followed by a 12-hour incubation with anti-βIG-H3 antibody (1:200 dilution) and 90 minutes with a goat anti-rabbit antibody conjugated to HRP. Normal rabbit IgG served as a control on tissue sections that were otherwise treated identically. Second antibody was localized by application of the chromagen DAB according to the manufacturer's recommendation. Sections were dehydrated and mounted with Permount for analysis by light microscopy. In some experiments, anti-βIG-H3 antibody was pre-absorbed with purified recombinant βIG-H3 in order to demonstrate antibody specificity.

Muscle was labeled by staining with anti-myosin monoclonal antibody F59 ENRfu (Crow and Stockdale, 1986). Mouse embyronic tissue sections (E17.5 and E18.5) were heated in 100 mM citrate buffer, pH 6.0 and irradiated for a total of eight minutes at 400W in a standard microwave. Staining with the anti-myosin antibody (1:10 dilution in PBS containing 1% BSA) and subsequent processing of tissue were performed essentially as described above.

Ultrastructural Analysis

Hind limbs from E17.5 mouse embryos were removed, rinsed in 230 milliosmole Sorenson's buffer pH 7.4 and fixed in 4% paraformaldehyde for 12 hours. Limbs were washed, blocked, immersed in a 1:4 solution of 30% hydrogen peroxide and methanol and blocked in 1.0% BSA for 12 hours. Tissue was incubated for 12 hours at 4° C. with anti-βIG-H3 (1:200) or an identical concentration of normal rabbit immunoglobulin, washed, and incubated an additional 12 hours at 4° C. with a goat anti-rabbit antibody conjugated to HRP. Second antibody was localized with DAB. Sections were washed in Sorenson's buffer and subsequently fixed in 1.0% osmium tetroxide. Samples were dehydrated in ethyl alcohol and propylene oxide, then embedded in Embed 812 and polymerized for 48 hours at 60° C. Sections 1-2 mm thick were stained with toluidine blue to assess orientation of the tissue utilizing optical microscopy. Once established, thin sections of 80-100 nm were cut on a Reichert Jung Ultracut E and examined for antibody localization. Selected sections were stained with saturated uranyl acetate or lead citrate for 5 minutes. Ultrastructural analysis was accomplished utilizing a JEOL 1230 transmission electron microscope. Digital images were collected with a Gatan Dual View camera interfaced with Photoshop and Plug-in Functions software.

Cell Culture

Chinese hamster ovary (CHO) cells expressing human recombinant βIG-H3 ENRfu (Skonier et al., 1994) were maintained in GMEM supplemented with 8% heat-treated and dialyzed FBS and βIG-H3 expression maintained under selective pressure by including 25 μM MSX in the culture medium. C2C12 murine myoblasts (ATCC CRL-1772) ENRfu (Yaffe and Saxel, 1977) were cultured as monolayers and maintained in DMEM supplemented with 10% heat-treated FBS. Primarymyoblasts were isolated from the quadricep muscle obtained from E17 mice and maintained as described ENRfu (Clegg et al., 1987). The purity of primary myoblast cells cultured as a monolayer was established by immunocytochemical detection of myosin fast chain using anti-myosin (F59) antibody. Prior to adhesion experiments, primary myoblasts were treated with 0.5 μg/ml cycloheximide as previously described ENRfu (Ettienne et al., 1981) and cycloheximide was included in all subsequent adhesion media.

Cell types were propagated in growth media supplemented with penicillin (50 μg/ml) and streptomycinsulfate (50 μg/ml) and maintained at 37° C. in a humidified atmosphere of95% ambient air and 5% CO₂. Cells were tested for mycoplasma by immunofluorescence and found negative. To assess whether βIG-H3 is upregulated by TGF-β1, cells were incubated at 37° C. for 24 hours with 20 ng/ml of TGF-β1. In these experiments, molecules greater than 30,000 daltons in the conditioned medium and control medium (normalized to cell number) were concentrated by microfiltration. Protein immunoblot analysis detected βIG-H3 utilizing anti-βIG-H3 antibody, a goat anti-rabbit second antibody conjugated to HRP, and DAB as a substrate.

Purification of Recombinant βIG-H3

When CHO cells expressing human recombinant βIG-H3 were estimated to be 70% confluent in roller bottles, then cells were rinsed three times with Hank's Balanced Salt solution. Subsequent maintenance of cells in serum-free GMEM for 48 hours provided conditioned medium for purification of βIG-H3. Serum-free conditioned medium was centrifuged to remove debris and the supernatant applied over a flow cell YM membrane (cutoff 30,000) to concentrate βIG-H3 in water. The retentate was lyophilized and stored at −20° C. until further use. Lyophilized material was rehydrated in 50 mM NaCl, 50 mM Tris buffer, pH 8.0, centrifuged and the supernatant applied sequentially over an anion exchange resin (BioRad Mono Q) followed by application onto hydroxyapatite and finally over a heparin-affinity resin. The composition of solutions applied in the purification process were as follows: Buffer A, 50 mM NaCl in 50 mM Tris pH 8.0; Buffer B, Buffer A containing 1 M NaCl; Buffer C, 10 mM NaPO₄ pH 6.8; Buffer D, 0.4 M NaPO₄ pH 6.8; Buffer E, 10 mM NaCl in 10 mM Tris pH 6.5 and Buffer F, comprised of Buffer E containing 1 M NaCl. Applications of Buffers A-F are described in the results section. The purity of βIG-H3 was assessed by SDS PAGE and immunoblot analysis.

Cell Attachment

Solutions of βIG-H3, type I collagen and fibronectin were prepared in PBS to a final concentration of 10 μg/ml. From these solutions, substrata were prepared by air-drying 0.1 ml onto wells of microtiter plates. A solution of 1% BSA in PBS served to make a negative control substratum. After coating, all wells were rinsed with PBS and any remaining exposed plastic surface was blocked for two hours with a solution of 1% BSA in PBS. To inhibit endogenous protein synthesis, cycloheximide was added to cells one hour before experiments and included in all subsequent adhesion buffers, the final concentration yielding 10 μg/ml. Cells detached from a monolayer using 1 mM EDTA were washed, suspended in serum-free DMEM and seeded at a density of 4×10⁴ cells/well. Following a 60-minute incubation at 37° C., non-attached cells were rinsed from the substrata. Addition of WST-1 and adsorption (405 nm) quantified the number of cells remaining attached. The recorded adsorption was compared to a standard curve obtained from a known number of C2C12 myoblasts. To document cell morphology, myoblasts that remained attached to various substrata were fixed with 10% formalin. Photomicrographs were taken using a Nikon Diaphot 200. Details of cell attachment experiments are provided in the individual figure legends. The specificity of cell attachment onto a substratum comprised of 30 μg/ml βIG-H3 was tested by pre-incubating cells with a solution containing suspended, soluble recombinant βIG-H3 prior to seeding. To determine whether the attachment of C2C12 myoblasts onto a βIG-H3 substratum was dependent on divalent cations, cells were incubated for 30 minutes in DMEM containing various concentrations of EDTA prior to seeding onto a substratum comprised of 10 μg/ml βIG-H3.

Function-perturbing anti-integrin antibodies were utilized to test for possible integrin-mediated attachment onto βIG-H3 substratum. The effects of antibodies to α1, α5, α6 and α7 and β1 were tested by pre-incubating myoblasts with dilutions of each antibody based on recommendations reported in the literature. Cycloheximide-treated C2C12 myoblasts were detached with 1 mM EDTA and incubated in suspension with anti-integrin antibodies for 30 minutes prior to seeding onto βIG-H3 and laminin substrata (10 μg/ml each of the respective proteins). After a 60-minute incubation at 37° C., non-attached cells were rinsed from wells and the number of cells remaining attached was quantified by recording adsorption 2 hours after addition of WST-1. To determine statistical significance for cell adhesion and inhibitory assays, a total of three experiments (duplicate wells in each individual experiment) ±S.D. were evaluated. Significance was calculated by paired t-tests. Differences were considered significant when p≦0.05.

Results

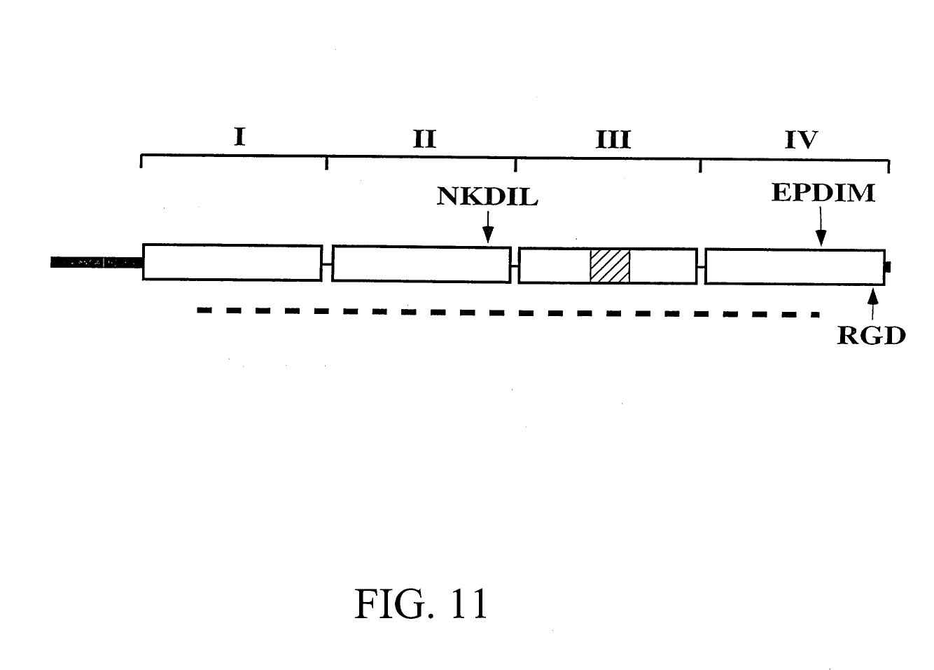

A primary structural feature of βIG-H3 includes four Fasciclin-like I repeats with regions of interdomain homology ENRfu (Skonier et al., 1992; Zinn et al., 1988) and that contain several peptide sequences that may bind ECM molecules and cells (FIG. 11). Our analysis revealed two possible heparin-binding sequences within the third fasciclin-like repeat (FIG. 11). These potential heparin-binding motifs may be functional as indicated by the use of a heparin-affinity column as a purification procedure for βIG-H3 (described below). An RGD tripeptide is located near the carboxy terminus of the fourth fasciclin-like repeat ENRfu (Skonier et al., 1992) and within the second and fourth fasciclin-like repeats respectively, the sequences NKDIL and EPDIM, reported to mediate adhesion of corneal epithelium cells ENRfu (Kim et al., 2000). The sequence of the βIG-H3 RNA probe used in the present study was tested for similarities with other molecules using the Basic Local Alignment Search Tool algorithm ENRfu (Altschul et al., 1990). Significant nucleotide similarities with other molecules in GenBank were not detected; therefore, the region indicated (dashed line, FIG. 11) was used to construct an antisense probe to test for expression of βIG-H3 transcripts in developing murine tissues.

βIG-H3 Transcripts and Protein Are Localized at Termini of Skeletal Muscle Fibers

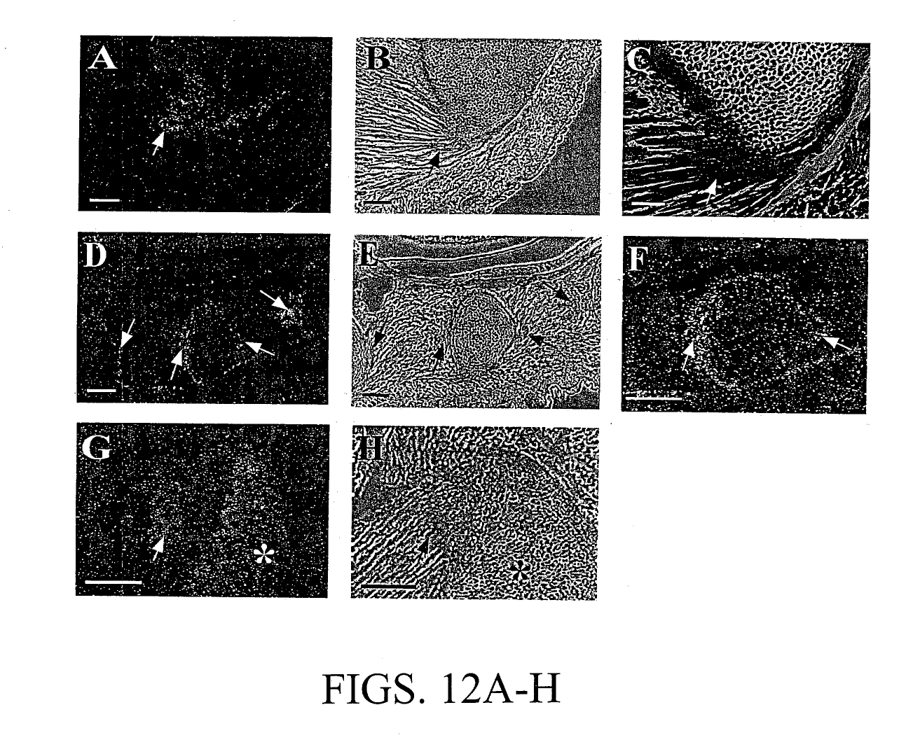

Beginning at E16.5 and observed through E18.5, representative dark field photo micrographs illustrate distinctive regions near primordial cartilage and bone that express a high density of βIG-H3 transcripts (FIGS. 12A, D, F and G). Transcripts were detected within and near areas where termini of skeletal muscle fibers contact developing connective tissue. Hybridized sections were also stained with methyl green for clarification of tissue integrity and for better comparison with darkfield images (FIGS. 12B, E and H). To better reveal muscle fibers and collagen, a representative section was treated with histology stains (FIG. 12C). Localization of βIG-H3 transcripts at regions where muscle fibers associate with developing cartilage and bone mass suggest that βIG-H3 is a component of MTJs. To test further for this possibility, an immunohistochemical analysis utilizing anti-βIG-H3 antibody was performed.

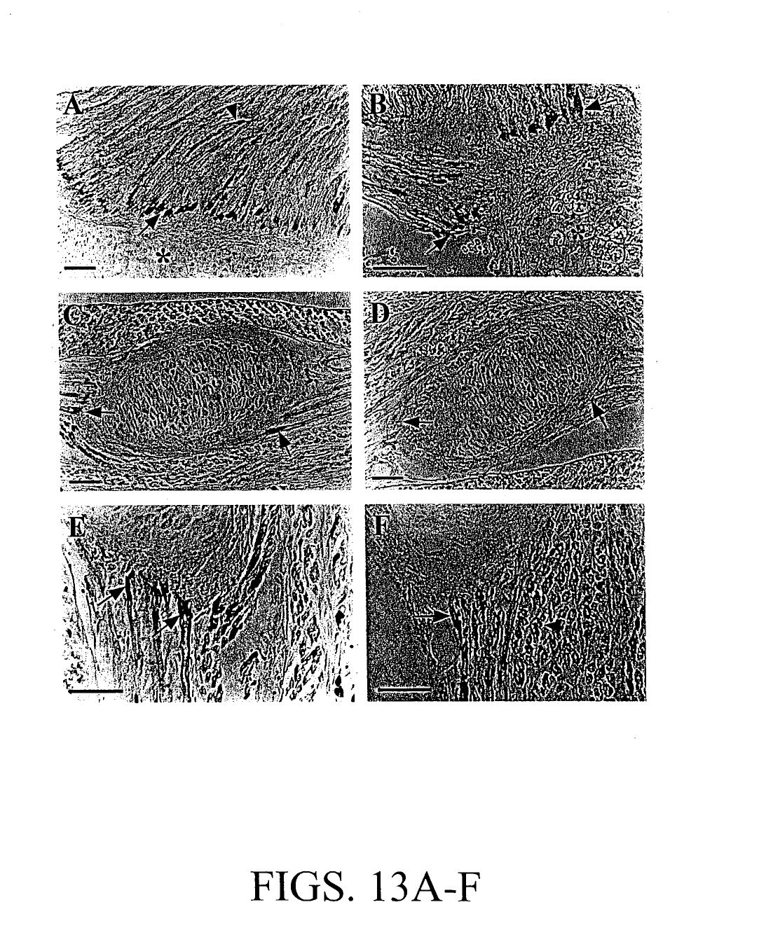

Anti-βIG-H3 anti body staining revealed that βIG-H3 is within MTJs. Staining was especially distinct at the termini of many skeletal muscle fibers where they juxtapose with tendon; for instance, where skeletal muscle fibers attached to developing femur (arrow, FIG. 13A). Within skeletal muscle, prominent staining was localized almost exclusively to the termini of muscle fibers, expect for occasional staining distal to MTJs (arrowhead, FIG. 13A). The distinctive staining pattern at the termini of myotubes was consistently observed in every tissue section that contained MTJs, albeit staining was not detected in every myotube. Shown are E18.5 developing facial bones, revealing the localization of βIG-H3 to numerous termini of developing skeletal muscle fibers (FIG. 13B) and developing rib, where βIG-H3 is detected at fibril termini (FIG. 13C). Antibody specificity was demonstrated by pre-incubating anti-βIG-H3 antibody with purified recombinant βIG-H3 prior to its application to tissue. An adjacent tissue section (FIG. 13D) incubated with the pre-absorbed antibody, and otherwise treated identically to tissue in FIG. 13(A-C), did not exhibit detectable staining. Photomicrographs of stained myotube terminals taken at increased magnification revealed that the anti-βIG-H3 staining coincided closely with the myotube contour rather than the tendon tissue itself (FIG. 13, E) and finally, an adjacent tissue section stained with F59 anti-myosin antibody demonstrates immunologically that anti-βIG-H3 antibody is localized to muscle fiber termini (FIG. 13F).

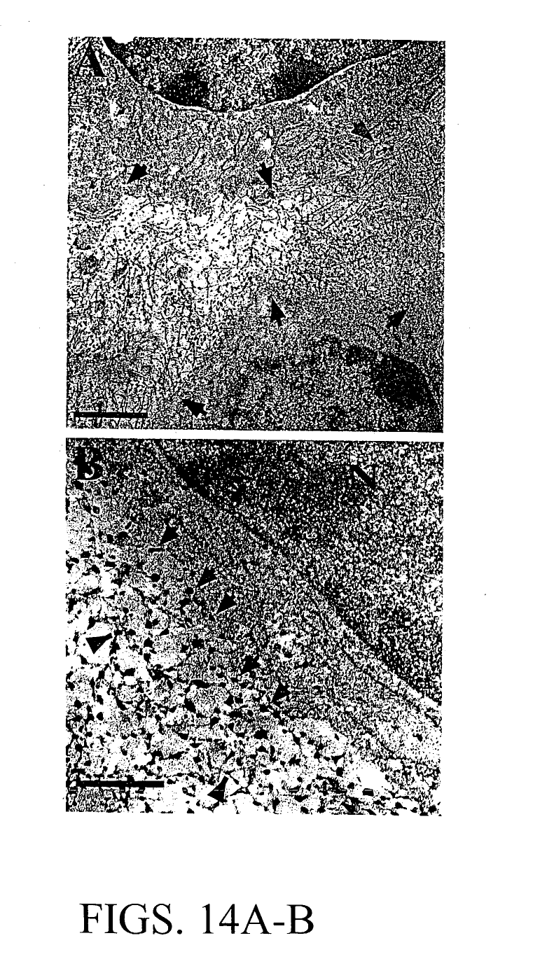

Ultrastructural analysis of MTJs in E17.5 mouse embryo hindlimb localized βIG-H3 antibody to fibers in the extracellular space surrounding myogenic cells (FIG. 14A). An increased magnification (FIG. 14B) illustrates that the anti-βIG-H3 antibody localized mostly, if not exclusively, with fibrils in the extracellular space and juxtaposed to cells identified as myoblasts based on cell fusion morphology and parallel groups of mononucleated, elongated cells as described ENRfu (Platzer, 1978).

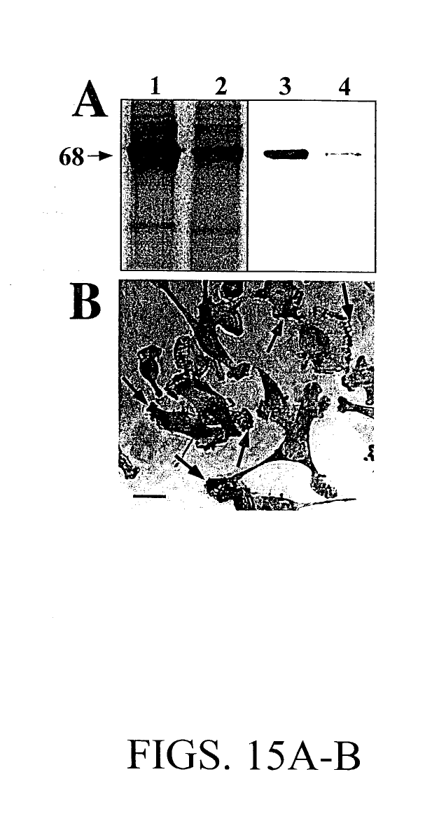

The localization of βIG-H3 at the extreme termini of myofibers (FIG. 13) and proximal to the cell surface (FIG. 14) suggests that βIG-H3 is synthesized by resident myoblasts. To begin to address this possibility, we investigated whether C2C12 cells expressed βIG-H3 and whether the expression was responsive to TGF-β1. Conditioned medium taken from C2C12 myoblasts that had been treated with 20 ng/ml TGF-β1 contained material that stained more intense with Coomassie Blue relative to conditioned medium taken from non-treated C2C12 cells (FIG. 15, lanes 1 and 2, respectively). Protein blot analysis utilizing anti-βIG-H3 antibody confirmed that a greater quantity of βIG-H3 was in the medium conditioned by C2C12 cells treated with TGF-β1 (FIG. 15, lanes 3 and 4). Interestingly, when C2C12 myoblasts were stained with anti-βIG-H3 antibody, then βIG-H3 localized at regions that appeared as short extensions and leading edges of the cell's of plasma membrane (FIG. 15B), suggesting βIG-H3 may function as an adhesion protein for skeletal muscle cells. To examine this possibility, recombinant βIG-H3 was expressed and purified.

Purification of Recombinant βIG-H3

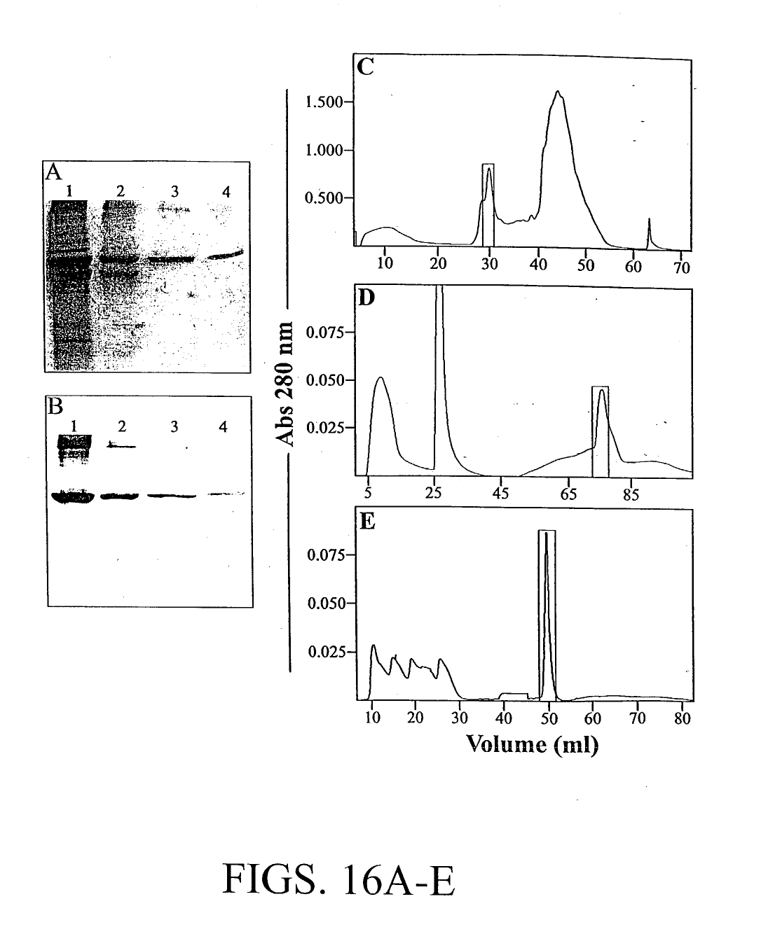

Because βIG-H3 contains putative heparin-binding sequences, the possibility it may bind heparin was tested utilizing column chromatography. Essentially all of the βIG-H3 in lyophilized starting material (see methods section) bound to a heparin-affinity column. This result was consistent with the possibility that the heparin-binding consensus sequences are functional in vitro and suggested heparin-affinity as a purification step. Indeed, a purification protocol was designed based on the heparin-binding property and on an estimated pI of βIG-H3 reported to range between 6.20 and 6.71 ENRfu (Escribano et al., 1994). Lyophilized starting material (FIG. 16A, lane 1) was re-hydrated in Buffer A and applied on an anion exchange resin equilibrated with Buffer A. Bound material was eluted with a linear gradient of Buffer B (FIG. 16A, lane 2) and fractions containing βIG-H3 pooled and applied over hydroxyapatite pre-equilibrated with Buffer A. The resin was washed with Buffer C and bound material eluted with a linear gradient of Buffer D (FIG. 16A, lane 3). Fractions containing βIG-H3 were pooled and applied over heparin-agarose pre-equilibrated with Buffer E. A linear salt gradient (Buffers E and F) eluted βIG-H3 and fractions containing βIG-H3 were pooled (FIG. 16, lane 4). A protein immunoblot containing material identical to that applied on the acrylamide gel (FIG. 16A, lanes 1-4) was stained with anti-βIG-H3 antibody (FIG. 16B, lanes 1-4), demonstrating that the chromatography series yielded a purified product. Shown in representative chromatographs are peaks corresponding to βIG-H3, eluted from anion exchange, hydroxyapatite and heparin agarose resins (FIGS. 16C, D, and E, respectively). Purified βIG-H3 was utilized as a substratum to test for possible cell binding activity.

Cell Attachment to βIG-H3

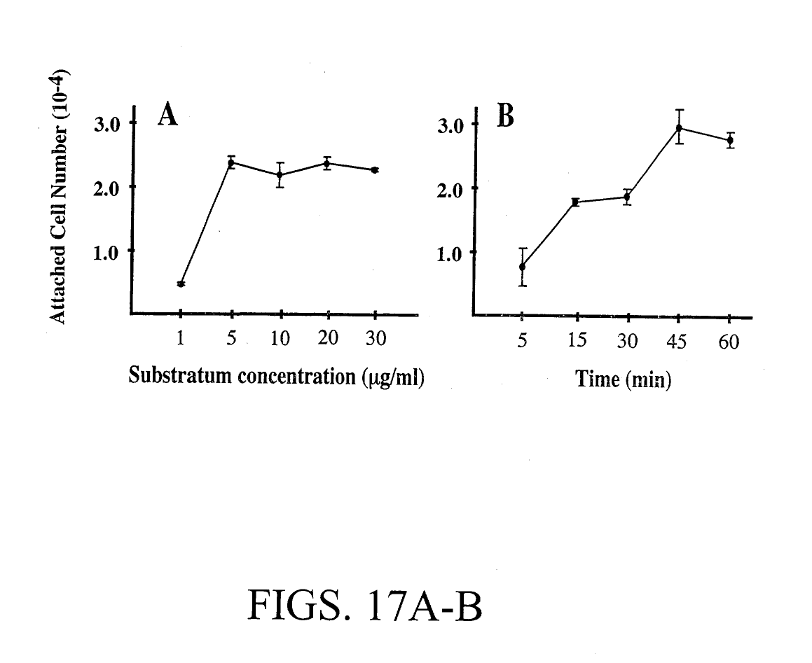

Our results demonstrate that in vitro, a substratum comprised ofβIG-H3 supports attachment and spreading of C2C12 murine myoblasts. A concentration of 5 μg/ml βIG-H3 was utilized to make a substratum that supported the attachment of 2.4×10⁴ cells (FIG. 17A). Cell attachment was also dependent on the time that cells were incubated on a βIG-H3 substratum. Approximately 2.8×10⁴ cells (70% of added cells) attached within 45 minutes on a substratum comprised of 10 μg/ml βIG-H3 (FIG. 17B). The attachment of cells on BSA after one hour was less than one percent of the added cells.

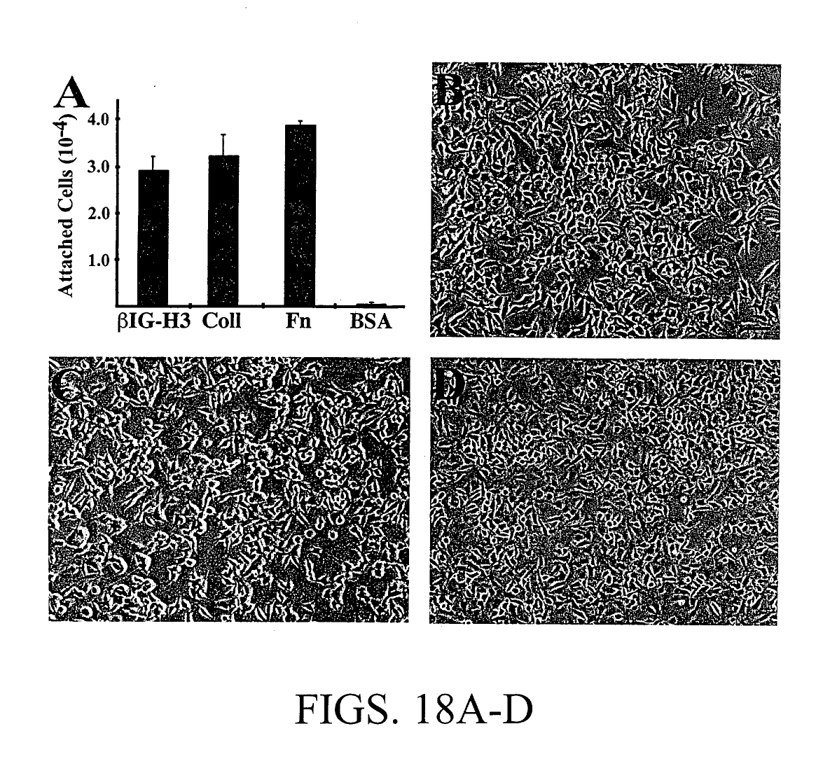

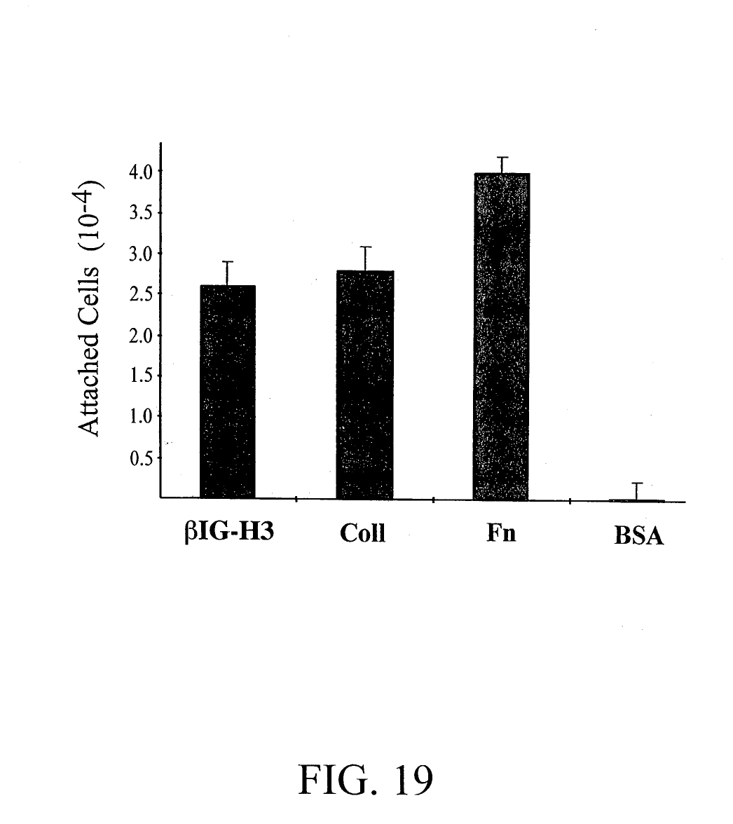

The number of cells attached on βIG-H3 and their spread morphology was similar to the adhesion promoted by type I collagen and fibronectin (FIGS. 18A-D). A substratum comprised of βIG-H3 supported the attachment of approximately 70% of seeded cells. Type I collagen supported an average of 84% of seeded cells and essentially all of the added cells attached to fibronectin. Cells on βIG-H3 were spread within 60 minutes (FIG. 18B). Similarly, cells attaching to type I collagen (FIG. 18C) and cells on fibronectin (FIG. 18D) showed a well-spread phenotype within 60 minutes, albeit occasional pockets of less well-spread cells were evident on collagen (FIG. 18C). Primary skeletal muscle cells seeded on βIG-H3, fibronectin and collagen attached similarly, the greatest number of cells adhering to fibronectin (FIG. 19).

Interestingly, adhesion of C2C12 cells onto a substratum comprised of βIG-H3 was reduced by 70% when the cells were pre-incubated for 10 minutes in DMEM containing βIG-H3. A pre-incubation time of 60 minutes prevented most, if not all cells from attaching to βIG-H3 (FIG. 120 A). When C2C12 cells pre-incubated for 30 minutes in medium containing βIG-H3, then their attachment to a substratum of βIG-H3 was significantly reduced in contrast to their adhesion onto fibronectin and laminin (FIGS. 120, C and D, respectively). The attachment of C2C12 myoblasts to βIG-H3 was also demonstrated to be dependent on divalent cations. Chelating with EDTA resulted in a reduction of approximately 90% of the number of cells attached to a βIG-H3 substratum (FIG. 120E).

Function-Perturbing Anti-Integrin Antibodies

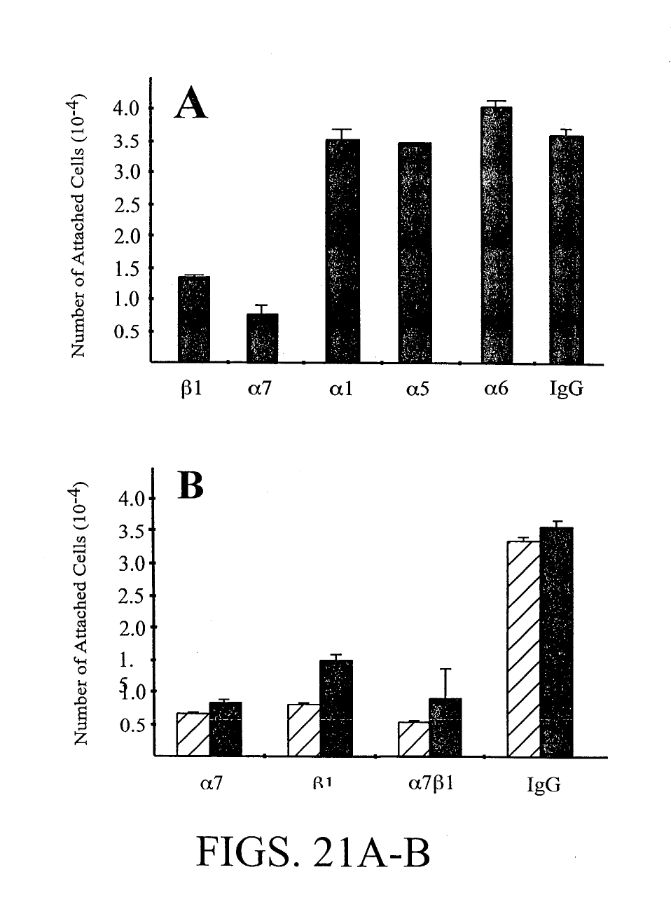

The possible activity of integrins as mediators of C2C12 cell attachment to βIG-H3 was investigated utilizing function-perturbing anti-integrin antibodies as described ENRfu (Yao et al., 1997). Included in the assessment were antibodies to the integrin subunits α1, α5, α6, α7 and β1 ENRfu (Garcia et al., 1999; Hirsch et al., 1994; Menko and Boettiger, 1987; Song et al., 1993; Sorokin et al., 2000). Either the anti-β1 antibody or the anti-α7 antibody, when mixed with C2C12 myoblasts, significantly reduced C2C12 cell attachment to βIG-H3 (FIG. 21A). Function-hindering anti-α and anti-β1 antibodies inhibited C2C12 myoblast attachment to laminin (FIG. 21B), a principal ligand for the α7β1 integrin ENRfu (Burkin and Kaufman, 1999).

Discussion

These studies document six novel findings. The first is that during murine development a prominent and decisive expression of βIG-H3 occurs where myofiber termini juxtapose to perichondrium. The second and third findings document that βIG-H3 within MTJs appears to associate with two components, fibrils within the intercellular space and pericellular material. The fourth discovery documents that βIG-H3 supports attachment of skeletal muscle cells in vitro and the fifth reveals that skeletal muscle cell adhesion onto a βIG-H3 substratum is mediated by the integrin α7β1. Finally, the sixth finding is that in vitro βIG-H3 binds heparin, suggesting that βIG-H3 may associate with proteoglycans. Collectively, this information is consistent with the possibility that βIG-H3 plays an adhesive and structural role in MTJs. ur in situ hybridization experiments evidence that βIG-H3 transcripts are markedly evident where muscle fiber termini juxtapose with developing bone, most prominently during the developmental stages E16.5-E18.5. Some of the more striking examples were observed where myosin-positive fibers approached the gross contour of femur head and rib perichondrium. The high density of transcripts in these regions suggests that βIG-H3 is synthesized by cells within the perichondrium and by myogenic cells. However, the exact perimeter between myofibril termini and perichondrium was not precisely defined in our in situ hybridization experiments, thus the possible expression of βIG-H3 mRNA by both myogenic cells and by the cells residing within the perichondrium was suggestive only.

To better delineate the spatial deposition of βIG-H3, we performed a series of immunohistochemical experiments. The results implicated skeletal muscle cells as the principal cell type associating with βIG-H3 in the MTJ. The deposition of βIG-H3 at MTJs was initially discerned in anti-βIG-H3 antibody stained paraffin embedded sections, because tendon noticeably appears as a condensation of mesenchymal cells at the termination of long appendage-like muscle cell processes ENRfu (Kardon, 1998) and βIG-H3 message and protein were observed to localize near and within tendon-muscle junctions. Sections stained with Masson's Trichrome chemically authenticated the presence of myofibril termini juxtaposed to collagen-rich tissue and confirm that these structures correspond to regions that express βIG-H3. However, whether βIG-H3 is synthesized in MTJs at myoblast termini exclusively is not clear. Cells residing within the perichondrium may synthesize βIG-H3 protein that subsequently associates with skeletal muscle cells. Precedence for such an organization has been documented where type IV collagen synthesized by tendon fibroblasts contributes to the developing basal lamina of myotubes ENRfu (Kuhl et al., 1984). Occasional discrete staining of βIG-H3 distal to muscle fiber termini was also evident. The staining may be various cell types that reside in skeletal muscle, including fibroblasts and satellite cells ENRfu (Mayne and Sanderson, 1985). Dermal fibroblasts express βIG-H3 in vitro and anti-βIG-H3 antibody was localized near or on cell bodies within human dermis ENRfu (LeBaron et al., 1995). However, the identity of structures stained along muscle fibers distal to the MTJ remains to be determined.

The prominent expression observed principally at myofibril termini suggests βIG-H3 associates with skeletal muscle cells. This possibility seems reasonable because βIG-H3 contains several peptide sequences that may serve as ligands for integrins ENRfu (Kim et al., 2000; Skonier et al., 1992). Additionally, biochemical evidence suggests that in vitro, βIG-H3 binds to ECM molecules including glycosaminoglycans (this study) and collagens ENRfu (Hashimoto et al., 1997; Rawe et al., 1997). To begin to investigate the subcellular distribution of βIG-H3 in MTJs, thin-sections of mouse hindlimb were examined utilizing immuno-TEM and anti-βIG-H3 antibody. The results revealed that βIG-H3 localized in close proximity to a meshwork of fibrils between cells and that βIG-H3 is associated with the skeletal muscle cells. This latter ultrastructural assessment is consistent with the observation that anti-βIG-H3 antibody localization coincides distinctively with digit-like extensions of skeletal muscle that protrude into the adjacent perichondrium. The presumed association of βIG-H3 with extracellular fiber-like molecules and cells suggest specific functions that βIG-H3 may mediate at myotube termini, where ECM molecules play important roles linking skeletal muscle to tendon.

To investigate whether βIG-H3 may play a biological role that involves an association with skeletal muscle cell-surface receptors, recombinant βIG-H3 was tested as a substratum for cell attachment. The cell adhesion experiments demonstrated that C2C12 cells and primary skeletal muscle cells similarly attached when seeded onto βIG-H3 substratum. Adherence of cells was detected on a substratum comprised of 1 μg/ml βIG-H3; however, a maximum number of added cells attached within 45 minutes onto a substratum formed of 10 μg/ml βIG-H3. Cell adhesion that is regulated by the time that cells are incubated on a substratum and by the concentration of molecules in the substratum is consistent with a receptor-mediated binding of cells to extracellular ligand. Pre-incubating myoblasts with soluble βIG-H3 revealed a time-dependent decrease in cell attachment to a βIG-H3 substratum. Thus βIG-H3 in solution appears to bind to its cell-surface receptor, consequently reducing the attachment of cells onto a βIG-H3 substratum. This finding suggests that a high-affinity binding for βIG-H3 occurs on the surface of myoblasts. Candidate sequences within βIG-H3 that may interact with cell surface receptors include the integrin recognition sequence RGD ENRfu (Pierschbacher and Ruoslahti, 1984) and perhaps either of two sequences comprised of NKDIL and EPDIM proposed to bind α3β1 ENRfu (Kim et al., 2000).

To understand whether skeletal muscle cell attachment onto βIG-H3 is mediated by integrins, conditions that antagonize integrin-mediated binding were introduced into our cell adhesion assays; the results implemented integrins as playing a role in C2C12 adhesion onto a βIG-H3 substratum. Myoblasts adhesion onto a βIG-H3 substratum was reduced by 84% by chelating divalent cations from the adhesion assay medium. Consistent with these results are previous studies that indicate myofiber adherence to the MTJ involves components of a divalent cation-dependent adhesion mechanism ENRfu (Law and Lightner, 1993). Additionally, function-perturbing anti-integrin antibodies reduced the number of cells that attached to a βIG-H3 substratum. A function-perturbing anti-α7 antibody reduced skeletal muscle attachment by approximately 80% whereas function-perturbing antibodies to the α5, α1 and α6 integrin subunits resulted in a slight reduction only.

The role of α7β1 in skeletal muscle cell adhesion onto a substratum comprised of βIG-H3 is consistent with the biology of this integrin in MTJs. The selective presence of α7β1 at MTJs implicated the α7 subunit as a determinant of junctional specificity ENRfu (Bao et al., 1993). Beginning at E14, α7β1 localized almost exclusively to the MTJ ENRfu (Bao et al., 1993) where it is proposed to play a role in MTJ organization by binding to laminin ENRfu (Miosge et al., 1999) and appears to be an essential link to ensure muscle integrity, particularly in regions subject to mechanical stress ENRfu (Yao et al., 1997). Thus, the anti-α7 antibody-mediated inhibition of skeletal muscle cells binding to βIG-H3 is compatible with the known expression of this integrin at MTJs and implicates βIG-H3 as a candidate ligand for α7β1 in vivo, suggesting βIG-H3 may also play a structural and organizational role in MTJs.

A separate cell-ECM interaction was also detected in vitro as βIG-H3 was shown to bind to heparin affinity resin. Most, if not all ECM adhesion glycoproteins that bind integrins exhibit affinity for glycosaminoglycan. Therefore, we examined βIG-H3 for heparin-binding consensus sequence. Such sequences are proposed to pattern X-B-B-X-B-X, where B is a basic amino acid and X is a hydropathic amino acid ENRfu (Cardin and Weintraub, 1989). The B-B-X-B pattern was utilized with MacVector version 6.5 sequence analysis software (Oxford Molecular Group, Madison, Wis.) to search for putative heparin-binding sequences in the CDNA deduced amino acid sequence of human βIG-H3. Two separate sequences that met the search criteria were discovered, suggesting βIG-H3 may bind glycosaminoglycans. Recombinant βIG-H3 binds heparin immobilized on agarose beads, signifying that βIG-H3 may interact with extracellular proteoglycans or proteoglycans associated with the cell-surface, the latter perhaps as a means to promote cell attachment. Cell-surface associated heparan sulfate proteoglycans serve as receptors for ECM proteins ENRfu (LeBaron et al., 1989), sometimes working in conjunction with integrins to form focal adhesions at cell-substratum contacts ENRfu (LeBaron et al., 1988; Woods et al., 1986). Dermal fibroblasts spread on a substratum comprised of recombinant βIG-H3 made focal adhesion-like plaques that stained with anti-phosphotyrosine antibody (unpublished observation), suggesting that glycosaminoglycan-binding activity within βIG-H3 is biologically functional in vitro, contributing to the process of cell-substratum adhesion and signaling processes. Heparan sulfate proteoglycans have been localized to developing MTJs ENRfu (Swasdison and Mayne, 1989) however, whether an interaction between βIG-H3 and glycosaminoglycan occurs within MTJs and any relevance to cell adhesion is not yet clear. Interestingly, anti-βIG-H3 antibody localized principally at the leading edges of C2C12 cells cultured as a monolayer. Isolated regions were stained, some appeared as punctum-like structures along the cell edge, suggesting that βIG-H3 is concentrated at the cell periphery and perhaps at the edges of extending plasma membrane. Whether there exists a co-localization of βIG-H3 and cell receptors, cytoskeleton or cytoplasmic components and whether βIG-H3 may play a role in cell movement is presently under investigation.

The observations we report implement βIG-H3 as an ECM molecule that supports α7β1-mediated attachment and spreading of skeletal muscle cells and as a protein that may associate with proteoglycans. TGF-β1 upregulated expression of βIG-H3 in cultured skeletal muscle cells; this result was not surprising because βIG-H3 is upregulated by TGF-β in many, but not all cell types ENRfu (Skonier et al., 1994; Skonier et al., 1992). The upregulation of βIG-H3 as a response to TGF-β1 suggests βIG-H3 may possibly play a role in regulating development and remodeling of MTJs ENRfu (Massague et al., 1986). Interestingly, a distinct expression pattern for βIG-H3 in the developing mouse localized to areas containing vast deposits of collagen. In addition to expression of βIG-H3 transcripts detected by in situ hybridization in cornea, sclera, choroid, and mesenchyme surrounding the optic stalk, ENRfu (Schorderet et al., 2000), we have also detected βIG-H3 in skeletal and smooth muscle, trigeminal ganglia, areas of endochondral and intramembranous ossification, tissue capsules, and areas of proliferating chondrocytes (work in progress). An emerging picture is that the spatiotemporal resemblances between expressions of collagen types I and II mRNA and βIG-H3 mRNA are similar during murine embryogenesis and may indicate a physiologically important interaction. This possibility is underscored by the findings that in vitro βIG-H3 binds collagen types I, II and IV ENRfu (Hashimoto et al., 1997) and was found co-isolated with type VI collagen ENRfu (Rawe et al., 1997). Collagen types I, II, and III are components of tendon ENRfu (Birk and Mayne, 1997; Cheah et al., 1991; Williams et al., 1980). Ultrastructural analysis has shown that collagen fibrils appear to emanate from tendon into the basal lamina of adjacent muscle fibers, possibly playing a role in the structural attachment of tendon to skeletal muscle fibers ENRfu (Mayne and Sanderson, 1985; Trelstad and Birk, 1984). The results of our immuno-TEM analysis suggest that βIG-H3 localizes near the cell, consistent with the possibility that βIG-H3 associates with cell surface receptors in vivo. Additionally, βIG-H3 localized to regions where fibrils appear to intersect in the extracellular space. The association of βIG-H3 with fibrillar and filamentous collagens ENRfu (Hashimoto et al., 1997; Rawe et al., 1997) and the considerable collagen content within MTJs suggests that the fiber-like material observed within the extracellular space of MTJs may be collagenous or possibly other proteins that may associate with βIG-H3 ENRfu (Gibson et al., 1996; Kitahama et al., 2000).

The biological activity of βIG-H3 presented here, together with the reported collagen-binding activity, suggest βIG-H3 secreted by myogenic cells during the formation of the MTJ may interact with skeletal muscle cells and with ECM molecules to form a macromolecular complex. Taken together, these findings confirm that βIG-H3 is a component of the ECM within the MTJ and that βIG-H3 may play a role mediating MTJ assembly during development. Studies investigating the adhesive structures that are formed by skeletal muscle cells attached to βIG-H3 and the exact identity of the fibers that βIG-H3 appears to associate with in the MTJ are ongoing.

Anti-βIG-H3 antibody also stained prominently muscle-tendon junctions in adult rat tissues (unpublished data), suggesting βIG-H3 plays important roles in adult MTJs. Interestingly, the βIG-H3 gene was mapped to human chromosome 5q31 ENRfu (Skonier et al., 1994). This region includes interval D5S1766-D5S178, proposed to contain genes that contribute to development of Limb Girdle Muscular Dystrophy Type 1A (LGMD 1A) ENRfu (Horrigan et al., 1999), an autosomal dominant dystrophy in males and females that begins in early adolescence or adulthood (Cohn, 2000). Progressive weakness in the hips and shoulder girdles, as well as absent or reduced tendon reflexes are clinical manifestations of this particular dystrophy. Thus, βIG-H3 is poised genetically and functionally as a candidate protein that may play a causative role in LGMD 1A. An investigation into a possible connection between βIG-H3 and LGMD1A and verification of its natural role as a protein linking skeletal muscle and tendon is currently being pursued.

### REFERENCES

Acsadi, G., G. Dickson, D. R. Love, A. Jani, F. S. Walsh, A. Gurusinghe, J. A. Wolff, and K. E. Davies. 1991. Human dystrophin expression in mdx mice after intramuscular injection of DNA constructs. Nature. 352:815-8.

Athanasiou, K. A., B. S. Thoma, D. R. Lanctot, D. Shin, C. M. Agrawal, and R. G. LeBaron. 1999. Development of the cytodetachment technique to quantify mechanical adhesiveness of the single cell. Biomaterials. 20:2405-15.

Bao, Z. Z., M. Lakonishok, S. Kaufmnan, and A. F. Horwitz. 1993. Alpha 7 beta 1 integrin is a component of the myotendinous junction on skeletal muscle. J Cell Sci. 106:579-89.

Bartoloni, L., S. K. Horrigan, K. D. Viles, J. M. Gilchrist, J. M. Stajich, J. M. Vance, L. H. Yamaoka, M. A. Pericak-Vance, C. A. Westbrook, and M. C. Speer. 1998. Use of a CEPH meiotic breakpoint panel to refine the locus of limb- girdle muscular dystrophy type 1A (LGMD 1A) to a 2-Mb interval on 5q31. Genomics. 54:250-5.

Belkin, A. M., S. F. Retta, O. Y. Pletjushkina, F. Balzac, L. Silengo, R. Fassler, V. E. Koteliansky, K. Burridge, and G. Tarone. 1997. Muscle beta 1D integrin reinforces the cytoskeleton-matrix link: modulation of integrin adhesive function by alternative splicing. J Cell Biol. 139:1583-95.

Belkin, A. M., N. I. Zhidkova, F. Balzac, F. Altruda, D. Tomatis, A. Maier, G. Tarone, V. E. Koteliansky, and K. Burridge. 1996. Beta 1D integrin displaces the beta 1A isoform in striated muscles: localization at junctional structures and signaling potential in nonmuscle cells. J Cell Biol. 132:211-26.

Bennett, A. M., and N. K. Tonks. 1997. Regulation of distinct stages of skeletal muscle differentiation by mitogen-activated protein kinases. Science. 278:1288-91.

Birk, D. E., and R. Mayne. 1997. Localization of collagen types I, III and V during tendon development. Changes in collagen types I and III are correlated with changes in fibril diameter. European Journal of Cell Biology. 72:352-61.

Blaschuk, K. L., and P. C. Holland. 1994. The regulation of alpha 5 beta 1 integrin expression in human muscle cells. Dev Biol. 164:475-83.

Bronner-Fraser, M., M. Artinger, J. Muschler, and A. F. Horwitz. 1992. Developmentally regulated expression of alpha 6 integrin in avian embryos. Development. 115:197-211.

Brus, I., and G. B. Glass. 1973. Estimation of cytotoxic injury to gastric parietal cells by trypan blue exclusion test followed by hematoxylin-eosin counterstaining of fixed smears. Stain Technol. 48:127-32.

Burkin, D. J., and S. J. Kaufinan. 1999. The alpha 7 beta 1 integrin in muscle development and disease. Cell Tissue Res. 296:183-90.

Cardin, A. D., and H. J. Weintraub. 1989. Molecular modeling of protein-glycosaminoglycan interactions. Arteriosclerosis. 9:21-32.

Cheah, K. S., E. T. Lau, P. K. Au, and P. P. Tam. 1991. Expression of the mouse alpha 1(II) collagen gene is not restricted to cartilage during development. Development. 111:945-53.

Choquet, D., D. P. Felsenfeld, and M. P. Sheetz. 1997. Extracellular matrix rigidity causes strengthening of integrin- cytoskeleton linkages. Cell. 88:39-48.

Clegg, C. H., T. A. Linkhart, B. B. Olwin, and S. D. Hauschka. 1987. Growth factor control of skeletal muscle differentiation: commitment to terminal differentiation occurs in G1 phase and is repressed by fibroblast growth factor. J Cell Biol. 105:949-56.

Clemens, P. R., S. Kochanek, Y. Sunada, S. Chan, H. H. Chen, K. P. Campbell, and C. T. Caskey. 1996. In vivo muscle gene transfer of full-length dystrophin with an adenoviral vector that lacks all viral genes. Gene Ther. 3:965-72.

Cohn, R. D., and K. P. Campbell. 2000. Molecular basis of muscular dystrophies. Muscle Nerve. 23:1456-71.

Crosbie, R. H., J. Heighway, D. P. Venzke, J. C. Lee, and K. P. Campbell. 1997. Sarcospan, the 25-kDa transmembrane component of the dystrophin- glycoprotein complex. J Biol Chem. 272:31221-4.

Crow, M. T., and F. E. Stockdale. 1986. Myosin expression and specialization among the earliest muscle fibers of the developing avian limb. Developmental Biology (Orlando). 113:238-54.

Davidson, B. L., S. E. Doran, D. S. Shewach, J. M. Latta, J. W. Hartman, and B. J. Roessler. 1994. Expression of Escherichia coli beta-galactosidase and rat HPRT in the CNS of Macaca mulatta following adenoviral mediated gene transfer. Exp Neurol. 125:258-67.

De Arcangelis, A., and E. Georges-Labouesse. 2000. Integrin and ECM functions: roles in vertebrate development. Trends Genet. 16:389-95.

Descamps, V., N. Blumenfeld, J. L. Villeval, W. Vainchenker, M. Perricaudet, and Y. Beuzard. 1994. Erythropoietin gene transfer and expression in adult normal mice: use of an adenovirus vector. Hum Gene Ther. 5:979-85.

Disatnik, M. H., and T. A. Rando. 1999. Integrin-mediated muscle cell spreading. The role of protein kinase c in outside-in and inside-out signaling and evidence of integrin cross-talk. J Biol Chem. 274:32486-92.

Drubin, D. G., S. C. Feinstein, E. M. Shooter, and M. W. Kirschner. 1985. Nerve growth factor-induced neurite outgrowth in PC12 cells involves the coordinate induction of microtubule assembly and assembly-promoting factors. J Cell Biol. 101:1799-807.

Duband, J. L., A. M. Belkin, J. Syfrig, J. P. Thiery, and V. E. Koteliansky. 1992. Expression of alpha 1 integrin, a laminin-collagen receptor, during myogenesis and neurogenesis in the avian embryo. Development. 116:585-600.

Edwards, R. J., and D. C. Watts. 1981. Specific spectrophotometric assay for the M isoenzymes of pyruvate kinase in plasma samples containing mixtures of the muscle (M) and liver (L) isoenzymes. Clin Chem. 27:906-9.

Ettinger, A. J., G. Feng, and J. R. Sanes. 1997. epsilon-Sarcoglycan, a broadly expressed homologue of the gene mutated in limb-girdle muscular dystrophy 2D. J Biol Chem. 272:32534-8.

Faulstich, H., S. Zobeley, G. Rinnerthaler, and J. V. Small. 1988. Fluorescent phallotoxins as probes for filamentous actin. J Muscle Res Cell Motil. 9:370-83.

Glogauer, M., P. Arora, D. Chou, P. A. Janmey, G. P. Downey, and C. A. McCulloch. 1998. The role of actin-binding protein 280 in integrin-dependent mechanoprotection. J Biol Chem. 273:1689-98.

Graham, F. L., and A. J. van der Eb. 1973. Transformation of rat cells by DNA of human adenovirus 5. Virology. 54:536-9.

Gu, M., W. Wang, W. K. Song, D. N. Cooper, and S. J. Kaufmnan. 1994. Selective modulation of the interaction of alpha 7 beta 1 integrin with fibronectin and laminin by L-14 lectin during skeletal muscle differentiation. J Cell Sci. 107:175-81.

Gussoni, E., G. K. Pavlath, A. M. Lanctot, K. R. Sharma, R. G. Miller, L. Steinman, and H. M. Blau. 1992. Normal dystrophin transcripts detected in Duchenne muscular dystrophy patients after myoblast transplantation. Nature. 356:435-8.

Haddada, H., T. Ragot, L. Cordier, M. T. Duffour, and M. Perricaudet. 1993. Adenoviral interleukin-2 gene transfer into P815 tumor cells abrogates tumorigenicity and induces antitumoral immunity in mice. Hum Gene Ther. 4:703-11.

Hashimoto, K., M. Noshiro, S. Ohno, T. Kawamoto, H. Satakeda, Y. Akagawa, K. Nakashima, A. Okimura, H. Ishida, T. Okamoto, H. Pan, M. Shen, W. Yan, and Y. Kato. 1997. Characterization of a cartilage-derived 66-kDa protein (RGD-CAP/beta ig-h3) that binds to collagen. Biochimica et Biophysica Acta. 1355:303-14.

He, T. C., S. Zhou, L. T. da Costa, J. Yu, K. W. Kinzler, and B. Vogelstein. 1998. A simplified system for generating recombinant adenoviruses. Proc Natl Acad Sci USA. 95:2509-14.

Heine, U., E. F. Munoz, K. C. Flanders, L. R. Ellingsworth, H. Y. Lam, N. L. Thompson, A. B. Roberts, and M. B. Spom. 1987. Role of transforming growth factor-beta in the development of the mouse embryo. J Cell Biol. 105:2861-76.

Helbling-Leclerc, A., X. Zhang, H. Topaloglu, C. Cruaud, F. Tesson, J. Weissenbach, F. M. Tome, K. Schwartz, M. Fardeau, K. Tryggvason, and et al. 1995. Mutations in the laminin alpha 2-chain gene (LAMA2) cause merosin- deficient congenital muscular dystrophy. Nat Genet. 11:216-8.

Hogervorst, F., I. Kuikman, E. Noteboom, and A. Sonnenberg. 1993. The role of phosphorylation in activation of the alpha 6A beta 1 laminin receptor. J Biol Chem. 268:18427-30.

Holt, K. H., L. E. Lim, V. Straub, D. P. Venzke, F. Duclos, R. D. Anderson, B. L. Davidson, and K. P. Campbell. 1998. Functional rescue of the sarcoglycan complex in the BIO 14.6 hamster using delta-sarcoglycan gene transfer. Mol Cell. 1:841-8.

Jaffe, H. A., C. Danel, G. Longenecker, M. Metzger, Y. Setoguchi, M. A. Rosenfeld, T. W. Gant, S. S. Thorgeirsson, L. D. Stratford-Perricaudet, M. Perricaudet, and et al. 1992. Adenovirus-mediated in vivo gene transfer and expression in normal rat liver. Nat Genet. 1:372-8.

Karsch-Mizrachl, I., M. Travis, H. Blau, and L. A. Leinwand. 1989. Expression and DNA sequence analysis of a human embryonic skeletal muscle myosin heavy chain gene. Nucleic Acids Res. 17:6167-79.

Kawamoto, T., M. Noshiro, M. Shen, K. Nakamasu, K. Hashimoto, Y. Kawashima-Ohya, O. Gotoh, and Y. Kato. 1998. Structural and phylogenetic analyses of RGD-CAP/beta ig-h3, a fasciclin-like adhesion protein expressed in chick chondrocytes. Biochim Biophys Acta. 1395:288-92.

Kim, J. E., S. J. Kim, B. H. Lee, R. W. Park, K. S. Kim, and I. S. Kim. 2000. Identification of motifs for cell adhesion within the repeated domains of transforming growth factor-beta -induced gene, beta ig-h3 [In Process Citation]. J Biol Chem. 275:30907-15.

Klintworth, G. K., Z. Valnickova, and J. J. Enghild. 1998. Accumulation of beta ig-h3 gene product in corneas with granular dystrophy. American Journal of Pathology. 152:743-8.

Korvatska, E., F. L. Munier, P. Chaubert, M. X. Wang, Y. Mashima, M. Yamada, S. Uffer, L. Zografos, and D. F. Schorderet. 1999. On the role of kerato-epithelin in the pathogenesis of 5q31-linked corneal dystrophies. Investigative Ophthalmology & Visual Science. 40:2213-9.

Kosher, R. A., W. M. Kulyk, and S. W. Gay. 1986. Collagen gene expression during limb cartilage differentiation. J Cell Biol. 102:1151-6.

Kuang, W., H. Xu, P. H. Vachon, L. Liu, F. Loechel, U. M. Wewer, and E. Engvall. 1998. Merosin-deficient congenital muscular dystrophy. Partial genetic correction in two mouse models. J Clin Invest. 102:844-52.

Kuhl, U., M. Ocalan, R. Timpl, R. Mayne, E. Hay, and K. von der Mark. 1984. Role of muscle fibroblasts in the deposition of type-IV collagen in the basal lamina of myotubes. Differentiation28:164-72.

Laemmli, U. K. 1970. Cleavage of structural proteins during the assembly of the head of bacteriophage T4. Nature. 227:680-5.

LeBaron, R. G., K.I. Bezverkov, M. P. Zimber, R. Pavelec, J. Skonier, and A. F. Purchio. 1995. Beta IG-H3, a novel secretory protein inducible by transforming growth factor-beta, is present in normal skin and promotes the adhesion and spreading of dermal fibroblasts in vitro. Journal of Investigative Dermatology. 104:844-9.

LeBaron, R. G., J. D. Esko, A. Woods, S. Johansson, and M. Hook. 1988. Adhesion of glycosaminoglycan-deficient chinese hamster ovary cell mutants to fibronectin substrata. Journal of Cell Biology. 106:945-52.

Lillie, R. D. 1940. Further experiments with the masson trichrome modification of mallory's connective tissue stain. Staining Technology. 15:17.

Mayer, U., G. Saher, R. Fassler, A. Bomemann, F. Echtermeyer, H. von der Mark, N. Miosge, E. Poschl, and K. von der Mark. 1997. Absence of integrin alpha 7 causes a novel form of muscular dystrophy. Nat Genet. 17:318-23.

Menko, A. S., and D. Boettiger. 1987. Occupation of the extracellular matrix receptor, integrin, is a control point for myogenic differentiation. Cell. 51:51-7.

Miller, A. D. 1990. Progress toward human gene therapy. Blood. 76:271-8.

Miosge, N., C. Klenczar, R. Herken, M. Willem, and U. Mayer. 1999. Organization of the myotendinous junction is dependent on the presence of alpha7beta1 integrin. Lab Invest. 79:1591-9.

Mitani, K., F. L. Graham, and C. T. Caskey. 1994. Transduction of human bone marrow by adenoviral vector. Hum Gene Ther. 5:941-8.

Morgan, J. E., E. P. Hoffmnan, and T. A. Partridge. 1990. Normal myogenic cells from newborn mice restore normal histology to degenerating muscles of the mdx mouse. J Cell Biol. 111 :2437-49.

Mosher, D. F. 1989. Fibronectin. Academic Press, New York.

Munier, F. L., E. Korvatska, A. Djemai, D. Le Paslier, L. Zografos, G. Pescia, and D. F. Schorderet. 1997. Kerato-epithelin mutations in four 5q3 1-linked corneal dystrophies [see comments]. Nature Genetics. 15:247-51.

Muroya, S., H. Takagi, S. Tajima, and A. Asano. 1994. Selective inhibition of a step of myotube formation with wheat germ agglutinin in a murine myoblast cell line, C2C12. Cell Struct Funct. 19:241-52.

O'Brien, E. R., K. L. Bennett, M. R. Garvin, T. W. Zderic, T. Hinohara, J. B. Simpson, T. Kimura, M. Nobuyoshi, H. Mizgala, A. Purchio, and S. M. Schwartz. 1996. Beta ig-h3, a transforming growth factor-beta-inducible gene, is overexpressed in atherosclerotic and restenotic human vascular lesions. Arteriosclerosis, Thrombosis & Vascular Biology. 16:576-84.

Ocalan, M., S. L. Goodman, U. Kuhl, S. D. Hauschka, and K. von der Mark. 1988. Laminin alters cell shape and stimulates motility and proliferation of murine skeletal myoblasts. Dev Biol. 125:158-67.

Partridge, T. A., J. E. Morgan, G. R. Coulton, E. P. Hoffrnan, and L. M. Kunkel. 1989. Conversion of mdx myofibres from dystrophin-negative to -positive by injection of normal myoblasts. Nature. 337:176-9.

Rando, T. A., and H. M. Blau. 1994. Primary mouse myoblast purification, characterization, and transplantation for cell-mediated gene therapy. J Cell Biol. 125:1275-87.

Rawe, I. M., Q. Zhan, R. Burrows, K. Bennett, and C. Cintron. 1997. Beta-ig. Molecular cloning and in situ hybridization in corneal tissues. Investigative Ophthalmology & Visual Science. 38:893-900.

Sanger, F., S. Nicklen, and A. R. Coulson. 1977. DNA sequencing with chain-terminating inhibitors. Proc Natl Acad Sci USA. 74:5463-7

Sicinski, P., Y. Geng, A. S. Ryder-Cook, E. A. Barnard, M. G. Darlison, and P. J. Barnard. 1989. The molecular basis of muscular dystrophy in the mdx mouse: a point mutation. Science. 244:1578-80.

Skonier, J., K. Bennett, V. Rothwell, S. Kosowski, G. Plowman, P. Wallace, S. Edelhoff, C. Disteche, M. Neubauer, H. Marquardt, and et al. 1994. beta ig-h3: a transforming growth factor-beta-responsive gene encoding a secreted protein that inhibits cell attachment in vitro and suppresses the growth of CHO cells in nude mice. DNA & Cell Biology. 13:571-84.

Song, W. K., W. Wang, R. F. Foster, D. A. Bielser, and S. J. Kaufman. 1992. H36-alpha 7 is a novel integrin alpha chain that is developmentally regulated during skeletal myogenesis. J Cell Biol. 117:643-57.

Song, W. K., W. Wang, H. Sato, D. A. Bielser, and S. J. Kaufmnan. 1993. Expression of alpha 7 integrin cytoplasmic domains during skeletal muscle development: alternate forms, conformational change, and homologies with serine/threonine kinases and tyrosine phosphatases. J Cell Sci. 106:1139-52.

Stratford-Perricaudet, L. D., M. Levrero, J. F. Chasse, M. Perricaudet, and P. Briand. 1990. Evaluation of the transfer and expression in mice of an enzyme-encoding gene using a human adenovirus vector. Hum Gene Ther. 1:241-56.

Streeten, B. W., Y. Qi, G. K. Klintworth, R. C. Eagle, Jr., J. A. Strauss, and K. Bennett. 1999. Immunolocalization of beta ig-h3 protein in 5q3 1-linked corneal dystrophies and normal corneas. Archives of Ophthalmology. 117:67-75.

Tidball, J. G. 1984. Myotendinous junction: morphological changes and mechanical failure associated with muscle cell atrophy. Experimental & Molecular Pathology. 40:1-12.

Tidball, J. G., and C. Lin. 1989. Structural changes at the myogenic cell surface during the formation of myotendinous junctions. Cell & Tissue Research. 257:77-84.

Tidball, J. G., M. J. Spencer, M. Wehling, and E. Lavergne. 1999. Nitric-oxide synthase is a mechanical signal transducer that modulates talin and vinculin expression. J Biol Chem. 274:33155-60.

Towbin, H., T. Stachelin, and J. Gordon. 1979. Electrophoretic transfer of proteins from polyacrylamide gels to nitrocellulose sheets: procedure and some applications. Proc Natl Acad Sci USA. 76:4350-4.

Trelstad, R. L., and D. E. Birk. 1984. Collagen fibril assembly at the surface of polarized cells. Liss, N.Y. 513-543 pp.

Trotter, J. A., S. Eberhard, and A. Samora. 1983. Structural connections of the muscle-tendon junction. Cell Motility. 3:431-8.

Vachon, P. H., H. Xu, L. Liu, F. Loechel, Y. Hayashi, K. Arahata, J. C. Reed, U. M. Wewer, and E. Engvall. 1997. Integrins (alpha7beta1) in muscle function and survival. Disrupted expression in merosin-deficient congenital muscular dystrophy. J Clin Invest. 100:1870-81.

Vincent, N., T. Ragot, H. Gilgenkrantz, D. Couton, P. Chafey, A. Gregoire, P. Briand, J. C. Kaplan, A. Kahn, and M. Perricaudet. 1993. Long-term correction of mouse dystrophic degeneration by adenovirus-mediated transfer of a minidystrophin gene. Nat Genet. 5:130-4.

Williams, I. F., A. Heaton, and K. G. McCullagh. 1980. Cell morphology and collagen types in equine tendon scar. Research in Veterinary Science. 28:302-10.

Wills, K. N., D. C. Maneval, P. Menzel, M. P. Harris, S. Sutjipto, M. T. Vaillancourt, W. M. Huang, D. E. Johnson, S. C. Anderson, S. F. Wen, and et al. 1994. Development and characterization of recombinant adenoviruses encoding human p53 for gene therapy of cancer. Hum Gene Ther. 5:1079-88.

Woods, A., J. R. Couchman, S. Johansson, and M. Hook. 1986. Adhesion and cytoskeletal organisation of fibroblasts in response to fibronectin fragments. EMBO Journal. 5:665-70.

Yaffe, D., and O. Saxel. 1977. Serial passaging and differentiation of myogenic cells isolated from dystrophic mouse muscle. Nature. 270:725-7.

Yao, C. C., J. Breuss, R. Pytela, and R. H. Kramer. 1997. Functional expression of the alpha 7 integrin receptor in differentiated smooth muscle cells. J Cell Sci. 110: 1477-87.

Ziober, B. L., Y. Chen, and R. H. Kramer. 1997. The laminin-binding activity of the alpha 7 integrin receptor is defined by developmentally regulated splicing in the extracellular domain. Mol Biol Cell. 8:1723-34.

Ziober, B. L., M. P. Vu, N. Waleh, J. Crawford, C. S. Lin, and R. H. Kramer. 1993. Alternative extracellular and cytoplasmic domains of the integrin alpha 7 subunit are differentially expressed during development. J Biol Chem. 268:26773-83.

### REFERENCES FROM Big-H3 EXPRESSION SECTION

Altschul, S. F., Gish, W., Miller, W., Myers, E. W. and Lipman, D. J. (1990). Basic local alignment search tool. Journal of molecular Biology 215, 403-10.

Barry-Lane, P. A., Wilson, S. E., Cavanagh, H. D., Petroll, W. M. and Jester, J. V. (1997). Characterization of SV40-transfected cell strains from rabbit keratocytes. Cornea 16, 72-8.

Billings, P. C., Herrick, D. J., Kucich, U., Engelsberg, B. N., Abrams, W. R., Macarak, E. J., Rosenbloom, J. and Howard, P. S. (2000). Extracellular matrix and nuclear localization of beta ig-h3 in human bladder smooth muscle and fibroblast cells. J Cell Biochem 79, 261-73.

Cardin, A. D. and Weintraub, H. J. (1989). Molecular modeling of protein-glycosaminoglycan interactions. Arteriosclerosis 9, 21-32.

Dieudonne, S. C., Kerr, J. M., Xu, T., Sommer, B., DeRubeis, A. R., Kuznetsov, S. A.,

Kim, I. S., Gehron Robey, P. and Young, M. F. (1999). Differential display of human marrow stromal cells reveals unique mRNA expression patterns in response to dexamethasone. Journal of Cellular Biochemistry 76, 231-43.

Escribano, J., Hernando, N., Ghosh, S., Crabb, J. and Coca-Prados, M. (1994). cDNA from human ocular ciliary epithelium homologous to beta ig-h3 is preferentially expressed as an extracellular protein in the corneal epithelium. Journal of Cellular Physiology 160, 511-21.

Fitzharris, T. P. and Markwald, R. R. (1982). Cellular migration through the cardiac jelly matrix: a stereoanalysis by high-voltage electron microscopy. Dev Biol 92, 315-29.

Ghosh-Choudhury, N., Windle, J. J., Koop, B. A., Harris, M. A., Guerrero, D. L., Wozney, J. M., Mundy, G. R. and Harris, S. E. (1996). Immortalized murine osteoblasts derived from BMP 2-T-antigen expressing transgenic mice. Endocrinology 137, 331-9.

Gibson, M. A., Hatzinikolas, G., Kumaratilake, J. S., Sandberg, L. B., Nicholl, J. K., Sutherland, G. R. and Cleary, E. G. (1996). Further characterization of proteins associated with elastic fiber microfibrils including the molecular cloning of MAGP-2 (MP25) [published erratum appears in J Biol Chem 1996 Mar 1 ; 271(9):5288]. Journal of Biological Chemistry 271, 1096-103.

Gilbert, R. E., Wilkinson-Berka, J. L., Johnson, D. W., Cox, A., Soulis, T., Wu, L. L., Kelly, D. J., Jerums, G., Pollock, C. A. and Cooper, M. E. (1998). Renal expression of transforming growth factor-beta inducible gene-h3 (beta ig-h3) in normal and diabetic rats [see comments]. Kidney International 54, 1052-62.

Hashimoto, K., Noshiro, M., Ohno, S., Kawamoto, T., Satakeda, H., Akagawa, Y., Nakashima, K., Okimura, A., Ishida, H., Okamoto, T. et al. (1997). Characterization of a cartilage-derived 6 6-kDa protein (RGD-CAP/beta ig-h3) that binds to collagen. Biochimica et Biophysica Acta 1355, 303-14.

Heine, U., Munoz, E. F., Flanders, K. C., Ellingsworth, L. R., Lam, H. Y., Thompson, N. L., Roberts, A. B. and Sporn, M. B. (1987). Role of transforming growth factor-beta in the development of the mouse embryo. J Cell Biol 105, 2861-76.

Hirano, K., Klintworth, G. K., Zhan, Q., Bennett, K. and Cintron, C. (1996). Beta ig-h3 is synthesized by corneal epithelium and perhaps endotheliumin Fuchs' dystrophic corneas. Current Eye Research 15, 965-72.

Horiuchi, K., Amizuka, N., Takeshita, S., Takamatsu, H., Katsuura, M., Ozawa, H., Toyama, Y., Bonewald, L. F. and Kudo, A. (1999). Identification and characterization of a novel protein, periostin, with restricted expression to periosteum and periodontal ligament and increased expression by transforming growth factor beta. Journal of Bone & Mineral Research 14, 1239-49.

Horrigan, S. K., Bartoloni, L., Speer, M. C., Fulton, N., Kravarusic, J., Ramesar, R., Vance, J. M., Yamaoka, L. H. and Westbrook, C. A. (1999). A radiation hybrid breakpoint map of the acute myeloid leukemia (AML) and limb-girdle muscular dystrophy 1A (LGMD1A) regions of chromosome 5q31 localizing 122 expressed sequences. Genomics 57, 24-35.

Hu, S., Sonnenfeld, M., Stahl, S. and Crews, S. T. (1998). Midline Fasciclin: a Drosophila Fasciclin-I-related membrane protein localized to the CNS midline cells and trachea. Journal of Neurobiology 35, 77-93.

Huber, O. and Sumper, M. (1994). Algal-CAMs: isoforms of a cell adhesion molecule in embryos of the alga Volvox with homology to Drosophila fasciclin I. EMBO Journal 13, 4212-22.

Icardo, J. M. and Manasek, F. J. (1984). An indirect immunofluorescence study of the distribution of fibronectin during the formation of the cushion tissue mesenchyme in the embryonic heart. Dev Biol 101, 336-45.

Kim, J. E., Kim, E. H., Han, E. H., Park, R. W., Park, I. H., Jun, S. H., Kim, J. C., Young, M. F. and Kim, I. S. (2000a). A TGF-beta-inducible cell adhesion molecule, betaig-h3, is down regulated in melorheostosis and involved in osteogenesis. Journal of Cellular Biochemistry 77, 169-78.

Kim, J. E., Kim, S. J., Lee, B. H., Park, R. W., Kim, K. S. and Kim, I. S. (2000b). Identification of motifs for cell adhesion within the repeated domains oftransforming growth factor-beta-induced gene, beta ig-h3 [In Process Citation]. J Biol Chem 275, 30907-15.

Korvatska, E., Munier, F. L., Chaubert, P., Wang, M. X., Mashima, Y., Yamada, M., Uffer, S., Zografos, L. and Schorderet, D. F. (1999). On the role of kerato-epithelin in the pathogenesis of 5q31-linked corneal dystrophies. Investigative Ophthalmology & Visual Science 40, 2213-9.

LeBaron, R. G., Bezverkov, K. I., Zimber, M. P., Pavelec, R., Skonier, J. and Purchio, A. F. (1995). Beta IG-H3, a novel secretory protein inducible by transforming growth factor-beta, is present in normal skin and promotes the adhesion and spreading of dermal fibroblasts in vitro. Journal of Investigative Dermatology 104, 844-9.

Munier, F. L., Korvatska, E., Djemai, A., Le Paslier, D., Zografos, L., Pescia, G. and Schorderet, D. F. (1997). Kerato-epithelin mutations in four 5q3 1-linked corneal dystrophies [see comments]. Nature Genetics 15, 247-51.

O'Brien, E. R., Bennett, K. L., Garvin, M. R., Zderic, T. W., Hinohara, T., Simpson, J. B., Kimura, T., Nobuyoshi, M., Mizgala, H., Purchio, A. et al. (1996). Beta ig-h3, a transforming growth factor-beta-inducible gene, is overexpressed in atherosclerotic and restenotic human vascular lesions. Arteriosclerosis, Thrombosis & Vascular Biology 16, 576-84.

Ohno, S., Noshiro, M., Makihira, S., Kawamoto, T., Shen, M., Yan, W., Kawashima-Ohya, Y., Fujimoto, K., Tanne, K. and Kato, Y. (1999). RGD-CAP ((beta)ig-h3) enhances the spreading of chondrocytes and fibroblasts via integrin alpha(1)beta(1). Biochimica et Biophysica Acta 1451, 196-205.

Pedersen, B. and Jensen, I. M. (1991). Clinical and prognostic implications of chromosome 5q deletions: 96 high resolution studied patients. Leukemia 5, 566-73.

Pierschbacher, M. D. and Ruoslahti, E. (1984). Variants of the cell recognition site of fibronectin that retain attachment-promoting activity. Proceedings of the National Academy of Sciences of the United States of America 81, 5985-8.

Rawe, I. M., Zhan, Q., Burrows, R., Bennett, K. and Cintron, C. (1997). Beta-ig. Molecular cloning and in situ hybridization in comeal tissues. Investigative Ophthalmology & Visual Science 38, 893-900.

Schorderet, D. F., Menasche, M., Morand, S., Bonnel, S., Buchillier, V., Marchant, D., Auderset, K., Bonny, C., Abitbol, M. and Munier, F. L. (2000). Genomic characterization and embryonic expression of the mouse Bigh3 (TGFβ1) gene. Biochem. Biophys. Res. Comm. 274,267-274.

Skonier, J., Bennett, K., Rothwell, V., Kosowski, S., Plowman, G., Wallace, P., Edelhoff, S., Disteche, C., Neubauer, M., Marquardt, H. et al. (1994). βeta ig-h3: a transforming growth factor-beta-responsive gene encoding a secreted protein that inhibits cell attachment in vitro and suppresses the growth of CHO cells in nude mice. DNA & Cell Biology 13, 571-84.

Skonier, J., Neubauer, M., Madisen, L., Bennett, K., Plowman, G. D. and Purchio, A. F. (1992). cDNA cloning and sequence analysis of beta ig-h3, a novel gene induced in a human adenocarcinoma cell line after treatment with transforming growth factor-beta. DNA & Cell Biology 11, 511-22.

Smith, P. K., Krohn, R. I., Hermanson, G. T., Mallia, A. K., Gartner, F. H., Provenzano, M. D., Fujimoto, E. K., Goeke, N. M., Olson, B. J. and Klenk, D. C. (1985). Measurement of protein using bicinchoninic acid. Anal Biochem 150, 76-85.

Streeten, B. W., Qi, Y., Klintworth, G. K., Eagle, R. C., Jr., Strauss, J. A. and Bennett, K. (1999). Immunolocalization of beta ig-h3 protein in 5q3 1-linked comeal dystrophies and normal corneas. Archives of Ophthalmology 117, 67-75.

Terasaka, K., Yamaguchi, R., Matsuo, K., Yamazaki, A., Nagai, S. and Yamada, T. (1989). Complete nucleotide sequence of immunogenic protein MPB70 from Mycobacterium bovis BCG. FEMS Microbiology Letters 49, 273-6.

Thompson, N. L., Flanders, K. C., Smith, J. M., Ellingsworth, L. R., Roberts, A. B. and Sporn, M. B. (1989). Expression of transforming growth factor-beta 1 in specific cells and tissues of adult and neonatal mice. J Cell Biol 108, 661-9.

Wheeler, E. F., Gong, H., Grimes, R., Benoit, D. and Vazquez, L. (1998). p75NTR and Trk receptors are expressed in reciprocal patterns in a wide variety of non-neural tissues during rat embryonic development, indicating independent receptor functions. J Comp Neurol 391, 407-28.

Yaffe, D. and Saxel, O. (1977). Serial passaging and differentiation of myogenic cells isolated from dystrophic mouse muscle. Nature 270, 725-7.

Zinn, K., McAllister, L. and Goodman, C. S. (1988). Sequence analysis and neuronal expression of fasciclin I in grasshopper and Drosophila. Cell 53, 577-87.

### REFERENCES FROM LOCALIZATION SECTION

ENBbu Altschul, S. F., W. Gish, W. Miller, E. W. Myers, and D. J. Lipman. 1990. Basic local alignment search tool. Journal of molecular Biology. 215:403-10.

Aumailley, M., R. Timpl, and A. Sonnenberg. 1990. Antibody to integrin alpha 6 subunit specifically inhibits cell-binding to laminin fragment 8. Exp Cell Res. 188:55-60.

Bao, Z. Z., M. Lakonishok, S. Kaufman, and A. F. Horwitz. 1993. Alpha 7 beta 1 integrin is a component of the myotendinous junction on skeletal muscle. J Cell Sci. 106:579-89.

Benjamin, M., and J. R. Ralphs. 2000. The cell and developmental biology of tendons and ligaments. Int Rev Cytol. 196:85-130.

Birk, D. E., and R. Mayne. 1997. Localization of collagen types I, III and V during tendon development. Changes in collagen types I and III are correlated with changes in fibril diameter. European Journal of Cell Biology. 72:352-61.

Burkin, D. J., and S. J. Kaufman. 1999. The alpha7beta1 integrin in muscle development and disease. Cell Tissue Res. 296:183-90.

Cardin, A. D., and H. J. Weintraub. 1989. Molecular modeling of protein-glycosaminoglycan interactions. Arteriosclerosis. 9:21-32.

Cheah, K. S., E. T. Lau, P. K. Au, and P. P. Tam. 1991. Expression of the mouse alpha 1(II) collagen gene is not restricted to cartilage during development. Development. 111:945-53.

Clegg, C. H., T. A. Linkhart, B. B. Olwin, and S. D. Hauschka. 1987. Growth factor control of skeletal muscle differentiation: commitment to terminal differentiation occurs in G1 phase and is repressed by fibroblast growth factor. J Cell Biol. 105:949-56.

Crow, M. T., and F. E. Stockdale. 1986. Myosin expression and specialization among the earliest muscle fibers of the developing avian limb. Developmental Biology (Orlando). 113:238-54.

Escribano, J., N. Hernando, S. Ghosh, J. Crabb, and M. Coca-Prados. 1994. cDNA from human ocular ciliary epithelium homologous to beta ig-h3 is preferentially expressed as an extracellular protein in the corneal epithelium. Journal of Cellular Physiology. 160:511-21.

Ettienne, E. M., K. Swartz, and R. H. Singer. 1981. Increased turnover of proteins from the sarcoplasmic reticulum of dystrophic chicken muscle cells in tissue culture. J Biol Chem. 256:6408-12.

Garcia, A . J., M. D. Vega, and D. Boettiger. 1999. Modulation of cell proliferation and differentiation through substrate- dependent changes in fibronectin conformation. Mol Biol Cell. 10:785-98.

Gibson, M. A., G. Hatzinikolas, J. S. Kumaratilake, L. B. Sandberg, J. K. Nicholl, G. R. Sutherland, and E. G. Cleary. 1996. Further characterization of proteins associated with elastic fiber microfibrils including the molecular cloning of MAGP-2 (MP25) [published erratum appears in J Biol Chem Mar. 1, 1996; 271(9):5288]. Journal of Biological Chemistry. 271:1096-103.

Hashimoto, K., M. Noshiro, S. Ohno, T. Kawamoto, H. Satakeda, Y. Akagawa, K. Nakashima, A. Okimura, H. Ishida, T. Okamoto, H. Pan, M. Shen, W. Yan, and Y. Kato. 1997. Characterization of a cartilage-derived 66-kD a protein (RGD-CAP/beta ig-h3) that binds to collagen. Biochimica et Biophysica Acta. 1355:303-14.

Hirano, K., G. K. Klintworth, Q. Zhan, K. Bennett, and C. Cintron. 1996. Beta ig-h3 is synthesized by corneal epithelium and perhaps endotheliumin Fuchs' dystrophic corneas. Current Eye Research. 15:965-72.

Hirsch, E., D. Gullberg, F. Balzac, F. Altruda, L. Silengo, and G. Tarone. 1994. Alpha v integrin subunit is predominantly located in nervous tissue and skeletal muscle during mouse development. Dev Dyn. 201:108-20.

J Horiuchi, K., N. Amizuka, S. Takeshita, H. Takamatsu, M. Katsuura, H. Ozawa, Y. Toyama, L. F. Bonewald, and A. Kudo. 1999. Identification and characterization of a novel protein, periostin, with restricted expression to periosteum and periodontal ligament and increased expression by transforming growth factor beta. Journal of Bone & Mineral Research. 14:1239-49.

Horrigan, S. K., L. Bartoloni, M. C. Speer, N. Fulton, J. Kravarusic, R. Ramesar, J. M. Vance, L. H. Yamaoka, and C. A. Westbrook. 1999. A radiation hybrid breakpoint map of the acute myeloid leukemia (AML) and limb-girdle muscular dystrophy 1A (LGMD1A) regions of chromosome 5q31 localizing 122 expressed sequences. Genomics. 57:24-35.

Hu, S., M. Sonnenfeld, S. Stahl, and S. T. Crews. 1998. Midline Fasciclin: a Drosophila Fasciclin-I-related membrane protein localized to the CNS midline cells and trachea. Journal of Neurobiology. 35:77-93.

Huber, O., and M. Sumper. 1994. Algal-CAMs: isoforms of a cell adhesion molecule in embryos of the alga Volvox with homology to Drosophila fasciclin I. EMBO Journal. 13:4212-22.

Kardon, G. 1998. Muscle and tendon morphogenesis in the avian hind limb. Development. 125:4019-32.

Kim, J. E., S. J. Kim, B. H. Lee, R. W. Park, K. S. Kim, and I. S. Kim. 2000. Identification of motifs for cell adhesion within the repeated domains of transforming growth factor-beta -induced gene, beta ig-h3 [In Process Citation]. J Biol Chem. 275:30907-15.

Kitahama, S., M. A. Gibson, G. Hatzinikolas, S. Hay, J. L. Kuliwaba, A. Evdokiou, G. J. Atkins, and D. M. Findlay. 2000. Expression of fibrillins and other microfibril-associated proteins in human bone and osteoblast-like cells. Bone. 27:61-7.

Kuhl, U., M. Ocalan, R. Timpl, R. Mayne, E. Hay, and K. von der Mark. 1984. Role of muscle fibroblasts in the deposition of type-IV collagen in the basal lamina of myotubes. Differentiation. 28:164-72.

Law, D. J., and V. A. Lightner. 1993. Divalent cation-dependent adhesion at the myotendinous junction: ultrastructure and mechanics of failure. Journal of Muscle Research & Cell Motility. 14:173-85.

LeBaron, R. G., K. I. Bezverkov, M. P. Zimber, R. Pavelec, J. Skonier, and A. F. Purchio. 1995. Beta IG-H3, a novel secretory protein inducible by transforming growth factor-beta, is present in normal skin and promotes the adhesion and spreading of dermal fibroblasts in vitro. Journal of Investigative Dermatology. 104:844-9.

LeBaron, R. G., J. D. Esko, A. Woods, S. Johansson, and M. Hook. 1988. Adhesion of glycosaminoglycan-deficient chinese hamster ovary cell mutants to fibronectin substrata. Journal of Cell Biology. 106:945-52.

LeBaron, R. G., A. Hook, J. D. Esko, S. Gay, and M. Hook. 1989. Binding of heparan sulfate to type V collagen. A mechanism of cell-substrate adhesion. Journal of Biological Chemistry. 264:7950-6.

Massague, J., S. Cheifetz, T. Endo, and B. Nadal-Ginard. 1986. Type beta transforming growth factor is an inhibitor of myogenic differentiation. Proc Natl Acad Sci USA. 83:8206-10.

Mayne, R., and R. D. Sanderson. 1985. The extracellular matrix of skeletal muscle. Collagen & Related Research. 5:449-68.

Mendrick, D. L., and D. M. Kelly. 1993. Temporal expression of VLA-2 and modulation of its ligand specificity by rat glomerular epithelial cells in vitro. Lab Invest. 69:690-702.

Menko, A. S., and D. Boettiger. 1987. Occupation of the extracellular matrix receptor, integrin, is a control point for myogenic differentiation. Cell. 51:51-7.

Miosge, N., C. Klenczar, R. Herken, M. Willem, and U. Mayer. 1999. Organization of the myotendinous junction is dependent on the presence of alpha7beta1 integrin. Lab Invest. 79:1591-9.

Miyake, S., T. Sakurai, K. Okumura, and H. Yagita. 1994. Identification of collagen and laminin receptor integrins on murine T lymphocytes. Eur J Immunol. 24:2000-5.

Ohno, S., M. Noshiro, S. Makihira, T. Kawamoto, M. Shen, W. Yan, Y. Kawashima-Ohya, K. Fujimoto, K. Tanne, and Y. Kato. 1999. RGD-CAP ((beta)ig-h3) enhances the spreading of chondrocytes and fibroblasts via integrin alpha(1)beta(1). Biochimica et Biophysica Acta. 1451:196-205.

Pierschbacher, M. D., and E. Ruoslahti. 1984. Variants of the cell recognition site of fibronectin that retain attachment-promoting activity. Proceedings of the National Academy of Sciences of the United States of America. 81:5985-8.

Platzer, A. C. 1978. The ultrastructure of normal myogenesis in the limb of the mouse. Anat Rec. 190:639-57.

Rawe, I. M., Q. Zhan, R. Burrows, K. Bennett, and C. Cintron. 1997. Beta-ig. Molecular cloning and in situ hybridization in corneal tissues. Investigative Ophthalmology & Visual Science. 38:893-900.

Schorderet, D. F., M. Menasche, S. Morand, S. Bonnel, V. Buchillier, D. Marchant, K. Auderset, C. Bonny, M. Abitbol, and F. L. Munier. 2000. Genomic characterization and embryonic expression of the mouse Bigh3 (Tgfbi) gene. Biochem. Biophys. Res. Comm. 274:267-274.

Schultz, J. F., and D. R. Armant. 1995. Beta 1- and beta 3-class integrins mediate fibronectin binding activity at the surface of developing mouse peri-implantation blastocysts. Regulation by ligand-induced mobilization of stored receptor. J Biol Chem. 270:11522-31.

Skonier, J., K. Bennett, V. Rothwell, S. Kosowski, G. Plowman, P. Wallace, S. Edelhoff, C. Disteche, M. Neubauer, H. Marquardt, and et al. 1994. beta ig-h3: a transforming growth factor-beta-responsive gene encoding a secreted protein that inhibits cell attachment in vitro and suppresses the growth of CHO cells in nude mice. DNA & Cell Biology. 13:571-84.

Skonier, J., M. Neubauer, L. Madisen, K. Bennett, G. D. Plowman, and A. F. Purchio. 1992. cDNA cloning and sequence analysis of beta ig-h3, a novel gene induced in a human adenocarcinoma cell line after treatment with transforming growth factor-beta. DNA & Cell Biology. 11:511-22.

Song, W. K., W. Wang, H. Sato, D. A. Bielser, and S. J. Kaufman. 1993. Expression of alpha 7 integrin cytoplasmic domains during skeletal muscle development: alternate forms, conformational change, and homologies with serine/threonine kinases and tyrosine phosphatases. J Cell Sci. 106:1139-52.

J Sorokin, L. M., M. A. Maley, H. Moch, H. von der Mark, K. von der Mark, L. Cadalbert, S. Karosi, M. J. Davies, J. K. McGeachie, and M. D. Grounds. 2000. Laminin alpha4 and integrin alpha6 are up regulated in regenerating dy/dy skeletal muscle: comparative expression of 1 aminin and integrin isoforms in muscles regenerating after crush injury. Exp Cell Res. 256:500-14.

Swasdison, S., and R. Mayne. 1989. Location of the integrin complex and extracellular matrix molecules at the chicken myotendinous junction. Cell & Tissue Research. 257:537-43.

Terasaka, K., R. Yamaguchi, K. Matsuo, A. Yamazaki, S. Nagai, and T. Yamada. 1989. Complete nucleotide sequence of immunogenic protein MPB70 from Mycobacterium bovis BCG. FEMS Microbiology Letters. 49:273-6.

Tidball, J. G. 1991. Force transmission across muscle cell membranes. Journal of Biomechanics. 24:43-52.

Tidball, J. G. 1994. Assembly of myotendinous junctions in the chick embryo: deposition of P68 is an early event in myotendinous junction formation. Developmental Biology (Orlando). 163:447-56.

Tidball, J. G., and C. Lin. 1989. Structural changes at the myogenic cell surface during the formation of myotendinous junctions. Cell & Tissue Research. 257:77-84.

Trelstad, R. L., and D. E. Birk. 1984. Collagen fibril assembly at the surface of polarized cells. Liss, N.Y. 513-543 pp.

Wheeler, E. F., H. Gong, R. Grimes, D. Benoit, and L. Vazquez. 1998. p75NTR and Trk receptors are expressed in reciprocal patterns in a wide variety of non-neural tissues during rat embryonic development, indicating independent receptor functions. J Comp Neurol. 391:407-28.

Williams, I. F., A. Heaton, and K. G. McCullagh. 1980. Cell morphology and collagen types in equine tendon scar. Research in Veterinary Science. 28:302-10.

Woods, A., J. R. Couchman, S. Johansson, and M. Hook. 1986. Adhesion and cytoskeletal organisation of fibroblasts in response to fibronectin fragments. EMBO Journal. 5:665-70.

Yaffe, D., and O. Saxel. 1977. Serial passaging and differentiation of myogenic cells isolated from dystrophic mouse muscle. Nature. 270:725-7.

Yao, C. C., J. Breuss, R. Pytela, and R. H. Kramer. 1997. Functional expression of the alpha 7 integrin receptor in differentiated smooth muscle cells. J Cell Sci. 110: 1477-87.

Zinn, K., L. McAllister, and C. S. Goodman. 1988. Sequence analysis and neuronal expression of fasciclin I in grasshopper and Drosophila. Cell. 53:577-87.

All references cited herein are incorporated by reference. While this invention has been described fully and completely, it should be understood that, within the scope of the appended claims, the invention may be practiced otherwise than as specifically described. Although the invention has been disclosed with reference to its preferred embodiments, from reading this description those of skill in the art may appreciate changes and modification that may be made which do not depart from the scope and spirit of the invention as described above and claimed hereafter.

We claim:

1. A composition comprising:
a therapeutically effective amount of βIG-H3, a βIG-H3 variant, a portion of βIG-H3, a variant of a portion βIG-H3 or mixtures thereof,
where the amount is sufficient to cure, treat, ameliorate, and/or prevent symptoms of Muscular Dystrophies or related neuromuscular diseases.

2. The composition of claim 1, wherein the composition comprises βIG-H3.

3. The composition of claim 1, wherein the composition comprises a portion of βIG-H3.

4. A composition comprising:
an amount of a DNA sequence encoding βIG-H3, a DNA sequence encoding a βIG-H3 variant, a DNA sequence encoding a portion of βIG-H3, a DNA sequence encoding a portion of a βIG-H3 variant, antisense sequences corresponding thereto, or mixtures thereof,
where the amount is sufficient to cause expression of the sequences in cells of an animal including a human to produce a therapeutically effective amount of encoded polypeptides sufficient to ameliorate, treat, prevent and/or cure Muscular Dystrophies or related neuromuscular diseases.

5. The composition of claim 4, wherein the composition comprises a DNA sequence encoding βIG-H3.

6. The composition of claim 4, wherein the composition comprises a DNA sequence encoding a portion of βIG-H3.

7. A plasmid comprising a DNA sequence encoding βIG-H3, a DNA sequence encoding a βIG-H3 variant, a DNA sequence encoding a portion of βIG-H3, a DNA sequence encoding a portion of a βIG-H3 variant, antisense sequences corresponding thereto, or mixtures thereof.

8. The plasmid of claim 7, wherein the plasmid elicits a therapeutic beneficial response to cure, treat, ameliorate, or prevent symptoms of Muscular Dystrophies or related neuromuscular diseases, when administered to an animal including a human in a therapeutically sufficient amount.

9. A DNA delivery system comprising a DNA sequence encoding βIG-H3, a DNA sequence encoding a βIG-H3 variant, a DNA sequence encoding a portion of βIG-H3, a DNA sequence encoding a portion of a βIG-H3 variant, antisense sequences corresponding thereto, or mixtures thereof, where the system is selected from the group consisting of a plasmid, a viral DNA delivery system, a liposome DNA delivery system and a mixtures thereof.

10. The DNA delivery system of claim 9, wherein the system elicits a therapeutic beneficial response to cure, treat, ameliorate, or prevent symptoms of Muscular Dystrophies or related neuromuscular diseases, when administered to an animal including a human in a therapeutically sufficient amount.

11. A method for treating Muscular Dstrophies or related neuromuscular diseases comprising the step of administering to a patient a therapeutically effective amount of a composition including βIG-H3, a βIG-H3 variant, a portion of βIG-H3, a variant of a portion βIG-H3 or mixtures thereof, where the amount it sufficient to reduce, prevent, cure, and/or treat symptoms associated with Muscular Dstrophies or related neuromuscular diseases.

12. The method of claim 11, wherein the administration is a periodic, where the period is less than a time required for the composition to no long reduce the symptoms associated with Muscular Dstrophies or related neuromuscular diseases.

13. The method of claim 11, wherein the administration is continuous.

14. A method for treating Muscular Dstrophies or related neuromuscular diseases comprising the step of administering to a patient a composition comprising a DNA sequence encoding βIG-H3, a DNA sequence encoding a βIG-H3 variant, a DNA sequence encoding a portion of βIG-H3, a DNA sequence encoding a portion of a βIG-H3 variant, antisense sequences corresponding thereto, or mixtures thereof in an amount sufficient to cause cells in the patient to express a translated polypeptide corresponding to the sequences at a therapeutically effective level to reduce, prevent, cure, ameliorate, and or treat symptoms of Muscular Dstrophies or related neuromuscular diseases.

15. The method of claim 14, wherein the composition further comprises a DNA delivery system selected from the group consisting of a plasmid, a viral delivery system, a liposome delivery system and mixtures thereof.

16. A method for delaying the onset of Muscular Dystrophies or related neuromuscular diseases comprising administering to a patient a composition comprising βIG-H3, a βIG-H3 variant, a portion of βIG-H3, a variant of a portion βIG-H3 or mixtures thereof according to a prophylactic treatment protocol sufficient to prevent or delay the onset of symptoms of Muscular Dystrophies or related neuromuscular diseases.

17. The method of claim 16, wherein the protocol comprises periodic administration of an amount of the composition at a level sufficient to prevent or delay the onset of symptoms of Muscular Dystrophies or related neuromuscular diseases.

18. The method of claim 17, wherein the period of the periodic administration is less than a time required for the composition to no long prevent or delay the onset of symptoms of Muscular Dystrophies or related neuromuscular diseases.

19. The method of claim 16, wherein the period of the periodic administration is between less than or equal to 1 day and less than or equal to six months.

20. The method of claim 16, wherein the protocol comprises continuous administration of an amount of the composition at a level sufficient to prevent or delay the onset of symptoms of Muscular Dystrophies or related neuromuscular diseases.


SEQUENCE LISTING (15 sequences)

SEQ ID NO: 1 - DNA, 2691 bp, Homo sapiens
  ref <301>: AUTHORS: Skonier,J., Neubauer,M., Madisen,L., Bennett,K., Plowman,G.D. and Purchio,A.F.
  ref <302>: TITLE: cDNA cloning and sequence analysis of beta ig-h3, a novel gene
  ref <303>: JOURNAL: DNA Cell Biol.
  ref <304>: VOLUME: 11
  ref <305>: ISSUE: 7
  ref <306>: PAGES: 511-522
  ref <307>: DATE: 1992
  ref <308>: DATABASE ACCESSION NUMBER: GenBank/M77349
  ref <309>: DATABASE ENTRY DATE: 1995-01-14
gcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctcctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 2 - DNA, 2674 bp, Mus musculus
  ref <301>: AUTHORS: Skonier,J., Bennett,K., Rothwell,V., Kosowski,S., Plowman,G., Wallace,P., Edelhoff,S., Disteche,C.M., Neubauer,M., Marquardt,H., Rodgers,J. and Purchio,A.F.
  ref <302>: TITLE: beta ig-h3: a transforming growth factor-beta-responsive gene encoding a secreted protein that inhibits cell attachment in vitro and suppresses the growth of CHO cells in nude mice
  ref <303>: JOURNAL: DNA Cell Biol.
  ref <304>: VOLUME: 13
  ref <305>: ISSUE: 6
  ref <306>: PAGES: 571-584
  ref <307>: DATE: 1994
  ref <308>: DATABASE ACCESSION NUMBER: GenBank/L19932
  ref <309>: DATABASE ENTRY DATE: 1994-08-15
ggcacgagcctgctttcatcgtgggtccgcgcgtgctccagctccatggcgctcctcatgcgactgctgaccctcgctctggcactgtctgtgggccccgctgggacccttgcaggtcccgccaagtcaccctaccagctggtgctgcagcatagccggctccggggtcgccagcacggccccaatgtatgtgctgtgcagaaggtcattggcaccaacaagaaatacttcaccaactgcaagcagtggtaccagaggaagatctgcggcaagtcgacagtcatcagttatgagtgctgtcctggatatgaaaaggtcccaggagagaaaggttgcccagcagctcttccgctctcaaatctgtatgagaccatgggagttgtgggatcgaccaccacacagctgtatacagaccgcacagaaaagctgaggcctgagatggagggacccggaagcttcaccatctttgctcctagcaatgaggcctggtcttccttgcctgcggaagtgctggactccctggtgagcaacgtcaacatcgaactgctcaatgctctccgctaccacatggtggacaggcgggtcctgaccgatgagctcaagcacggcatgaccctcacctccatgtaccagaattccaacatccagatccatcactatcccaatgggattgtaactgttaactgtgcccggctgctgaaggctgaccaccatgcgaccaacggcgtggtgcatctcattgacaaggtcatttccaccatcaccaacaacatccagcagatcattgaaatcgaggacacctttgagacacttcgggccgccgtggctgcatcaggactcaataccgtgctggagggcgacggccagttcacactcttggccccaaccaacgaggcctttgagaagatccctgccgagaccttgaaccgcatcctgggtgacccagaggcactgagagacctgctaaacaaccacatcctgaagtcagccatgtgtgctgaggccattgtagctggaatgtccatggagaccctggggggcaccacactggaggtgggctgcagtggggacaagctcaccatcaacgggaaggctgtcatctccaacaaagacatcctggccaccaacggtgtcattcatttcattgatgagctgcttatcccagattcagccaagacactgcttgagctggctggggaatctgacgtctccactgccattgacatcctcaaacaagctggcctcgatactcatctctctgggaaagaacagttgaccttcctggcccccctgaattctgtgttcaaagatggtgtccctcgcatcgacgcccagatgaagactttgcttctgaaccacatggtcaaagaacagttggcctccaagtatctgtactctggacagacactggacacgctgggtggcaaaaagctgcgagtctttgtttatcgaaatagcctctgcattgaaaacagctgcattgctgcccatgataagaggggacggtttgggaccctgttcaccatggaccggatgttgacacccccaatggggacagttatggatgtcctgaagggagacaatcgttttagcatgctggtggccgccatccagtctgcaggactcatggagatcctcaaccgggaaggggtctacactgtttttgctcccaccaatgaagcgttccaagccatgcctccagaagaactgaacaaactcttggcaaatgccaaggaacttaccaacatcctgaagtaccacattggtgatgaaatcctggttagcggaggcatcggggccctggtgcggctgaagtctctccaaggggacaaactggaagtcagctcgaaaaacaatgtagtgagtgtcaataaggagcctgttgccgaaaccgacatcatggccacaaacggtgtggtctatgccatcaacactgttctgcagccgccagccaaccgaccacaagaacgaggagatgagctggcagactctgcccttgaaatcttcaaacaggcgtcagcgtattccagggctgcccagaggtctgtgcgacttgcccctgtctatcagcggttactggagaggatgaagcattagcaggaagaccgaggaggagagccctgcagcagcttcccgccagtttctctcagtttgccaaagagaccattgaatgtttttgaaaccaaagagcacacttcaacatacatgggcgcaccatattgagatctgagccttggacgggtagggaaggggttaaggggagaaaggttctttttagctttgatccctccaaaccgtggttgttaacccattcgaatatacagatctggcagtcatagcttggcaccaaattcccgaaagacctctcgaaagcatgaatttcctgactgtgccaaggcctgataaagggaactacggcatcttggagctcacaaatgtgaatcaagcagtccggcattctggaaagccttggcatggttctgtaaagctcttgtaccgctggagaaacggcatcactataagctatgagttgaactgtttctgtcaagtatgtcttgtgtccacacatggtttggatgcttctatattggccctgcccaggtagaaagggtaagaagaacatgtagaatccagattccctgagtgtgagggacccatggtgcatttgtaataa

SEQ ID NO: 3 - DNA, 2220 bp, Oryctolagus cuniculus
atggcgctcttcgtgcggctgctggctctcgccctggctctggcttgggccccgccgcgaccctggccggccccgccaagtctccctaccagctggtactccagcatagccggctccgccgccagcagcacggccccaacgtgtgcgctgtgcagaaggtcatcggcaccaacaggaagtacttcaccaactgcaagcagtggtaccagaggaaaatctgtggcaaatcaaccgtcatcagctacgagtgctgtcctggctatgaaaaggtccccggggagagaagctgtccagcagccctcccactcgccaacctctacgagaccctgggggttgttggatcgaccaccacccagctgtacacagaccgcacggagaaactgaggcctgagatggaggggcccggccgattcaccatcttcgcccccagcaacgaggcctgggcttccttgccagcggaggtgctggactccctggtgagcaacgtcaacatcgagctgctcaacgccctgcgctaccacatggtggaccgccgggtcctcaccgacgagctgaagcacggcatggccctcacctccatgtaccagaactccaaattccagatccaccactatcccaacgggatcgtgaccgtgaactgcgcccggctgctgaaggccgaccaccatgccaccaacggcgtggtgcacctcatcgacaaggtcatctccactgtcaccaacaacatccagcagatcatcgagatcgaggacacctttgagaccctgcgggctgccgtggccgcatcggggctcaacaccctgctcgagagtgatggccagttcacgctcttggccccaaccaacgaggccaaagagaagatccctactgagactttgaaccggatcttgggtgatccagaggccctgagagacctgctgaacaaccacatcctgaagtcagccatgtgtgctgaagccattgtcgccgggctgtccatggagaccctggaggccaccacactggaggtgggctgcagcggggacatgctcaccatcaacggcaaggccatcatctccaataaagacgtcttggccaccaacggtgtcattcacttcatcgatgagctgctcatccccgactccgccaagacgctgtctgagctggctgcaggatccgacgtctccacggccatcgaccttttcggacaagctggcctcggcactcacctctctggaaatgagcggctcaccctgctggcccccctgaattctgtgttcgaagaaggagcccctccaattgatgcccatacaaggaatttgcttcggaaccacataattaaagaccagctggcctctaagtatctgtaccatggacagaccctggacacgctgggaggcaaaaagctgagagtttttgtttatcgtaacagcctgtgcatcgagaacagttgcatcgctgcccatgacaagagggggaggtacgggacgctgttcaccatggaccggatgctgacgccccccagtggcaccgtcatggacgtcttgaagggggacaaccgctttagcatgctggtggccgccatccagttccgcaggctgactgagaccctcaaccgggaaggggcctacactgtcttcgctcccaccaacgaagccttccaagccctgccaccaggagagctgaacaaactgttgggaaatgccaaggaacttgccgacatcctgaaataccatgtgggcgaagaaatcctggtgagcgggggcatcgggaccctggtgcggctgaagtccctccagggcgacaagctagaagtcagctcgaaaaacaatgcggtgagtgtcaacaaggagcctgttgctgaaagtgacatcatggccacaaatggcgtggtctatgccatcaccagcgttctgcagcctccagccaacagacctcaggaacgaggggatgaacttgcagactctgcgcttgagatcttcaaacaagcgtcggcgttttccagggcttcccagaggtctgtgcgactagcccctgtctatcagaggctattggaaaggatgaagcactaacgcagcagaccacaggaggaaggcaccgtggcagctgcccaccagcatctttgtttgccaaagagactgttttggaaaccaaatatcacccttcagtgtacatggcccgcaccctaatgagacctgagcctggggcagtgggggcaggagggagagaagtctttatttt

SEQ ID NO: 4 - protein, 682 aa, Homo sapiens
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 5 - protein, 682 aa, Oryctolagus cuniculus
GALRAAAGSRPGSGLGPAATLAGPAKSPYQLVLQHSRLRRQQHGPNVCAVQKVIGTNRKYFTNCKQWYQRKICGKSTVISYECCPGYEKVPGERSCPAALPLANLYETLGVVGSTTTQLYTDRTEKLRPEMEGPGRFTIFAPSNEAWASLPAEVLDSLVSNVNIELLNALRYHMVDRRVLTDELKHGMALTSMYQNSKFQIHHYPNGIVTVNCARLLKADHHATNGVVHLIDKVISTVTNNIQQIIEIEDTFETLRAAVAASGLNTLLESDGQFTLLAPTNEAKEKIPTETLNRILGDPEALRDLLNNHILKSAMCAEAIVAGLSMETLEATTLEVGCSGDMLTINGKAIISNKDVLATNGVIHFIDELLIPDSAKTLSELAAGSDVSTAIDLFGQAGLGTHLSGNERLTLLAPLNSVFEEGAPPIDAHTRNLLRNHIIKDQLASKYLYHGQTLDTLGGKKLRVFVYRNSLCIENSCIAAHDKRGRYGTLFTMDRMLTPPSGTVMDVLKGDNRFSMLVAAIQFRRLTETLNREGAYTVFAPTNEAFQALPPGELNKLLGNAKELADILKYHVGEEILVSGGIGTLVRLKSLQGDKLEVSSKNNAVSVNKEPVAESDIMATNGVVYAITSVLQPPANRPQERGDELADSALEIFKQASAFSRASQRSVRLAPVYQRLLERMKH

SEQ ID NO: 6 - protein, 683 aa, Mus musculus
MALLMRLLTLALALSVGPAGTLAGPAKSPYQLVLQHSRLRGRQHGPNVCAVQKVIGTNKKYFTNCKQWYQRKICGKSTVISYECCPGYEKVPGEKGCPAALPLSNLYETMGVVGSTTTQLYTDRTEKLRPEMEGPGSFTIFAPSNEAWSSLPAEVLDSLVSNVNIELLNALRYHMVDRRVLTDELKHGMTLTSMYQNSNIQIHHYPNGIVTVNCARLLKADHHATNGVVHLIDKVISTITNNIQQIIEIEDTFETLRAAVAASGLNTVLEGDGQFTLLAPTNEAFEKIPAETLNRILGDPEALRDLLNNHILKSAMCAEAIVAGMSMETLGGTTLEVGCSGDKLTINGKAVISNKDILATNGVIHFIDELLIPDSAKTLLELAGESDVSTAIDILKQAGLDTHLSGKEQLTFLAPLNSVFKDGVPRIDAQMKTLLLNHMVKEQLASKYLYSGQTLDTLGGKKLRVFVYRNSLCIENSCIAAHDKRGRFGTLFTMDRMLTPPMGTVMDVLKGDNRFSMLVAAIQSAGLMEILNREGVYTVFAPTNEAFQAMPPEELNKLLANAKELTNILKYHIGDEILVSGGIGALVRLKSLQGDKLEVSSKNNVVSVNKEPVAETDIMATNGVVYAINTVLQPPANRPQERGDELADSALEIFKQASAYSRAAQRSVRLAPVYQRLLERMKH

SEQ ID NO: 7 - protein, 5 aa, Homo sapiens
NKDIL

SEQ ID NO: 8 - protein, 5 aa, Homo sapiens
EPDIM

SEQ ID NO: 9 - protein, 3 aa, Homo sapiens
RGD

SEQ ID NO: 10 - protein, 4 aa, Homo sapiens
KKLR

SEQ ID NO: 11 - protein, 4 aa, Homo sapiens
KRGR

SEQ ID NO: 12 - DNA, 22 bp, RT-PCR primer
tgcccgtcggtcgcaagcttgc

SEQ ID NO: 13 - DNA, 21 bp, RT PCR primer
tgtagtgcttcaagcttatgc

SEQ ID NO: 14 - DNA, 22 bp, Sense PCR primer
cgaactgctcaatgctctccgc

SEQ ID NO: 15 - DNA, 20 bp, RT-PCR primer
ccccgatgcctccgctaacc